(19) United States
(12) Patent Application Publication
(10) Pub. No.: US 2016/0259883 A1
(43) Pub. Date: Sep. 8, 2016

(54) SENSE-ANTISENSE GENE PAIRS FOR PATIENT STRATIFICATION, PROGNOSIS, AND THERAPEUTIC BIOMARKERS IDENTIFICATION

(71) Applicant: Agency for Science, Technology and Research, Singapore (SG)

(72) Inventors: Oleg GRINCHUK, Singapore (SG); Efthimios MOTAKIS, Singapore (SG); Surya Pavan YENAMANDRA, Singapore (SG); Vladimir Andreevich KUZNETSOV, Singapore (SG)

(21) Appl. No.: 15/030,370

(22) PCT Filed: Oct. 20, 2014
(86) PCT No.: PCT/SG2014/000492

§ 371 Date: Apr. 18, 2016

(30) Foreign Application Priority Data
Oct. 18, 2013 (SG) 201307917-3

(51) Int. Cl. G06F 19/20; C40B 30/02; C12Q 1/68

(52) U.S. Cl. CPC G06F 19/20; C12Q 1/6886; C40B 30/02; C12Q 2600/118; C12Q 2600/158; C12Q 2600/106

(57) ABSTRACT

The present invention relates to a method of identification of clinically and genetically distinct sub-groups of patients subject to a medical condition, particularly breast, lung, and colon cancer patients using a composition of respective gene expression values for certain gene pairs. Sense-antisense gene pairs (SAGPs) which are relevant for a medical condition and the disease prognosis are used by the method to generate statistical models based on the expression values of the SAGPs. SAGPs for which the statistical models are found to have high value in prognosis of the variation of medical condition and the diseases are selected and integrated in the prognostic signature including specified parameters (e.g. cut-off values) of the prognostic model. It further relates to using respective gene expression values for these genes to predict patient′ risk groups (in context of patient's survival or/and disease progression) and to using the predicted groups for identification of patient risk, and specific and robust prognostic biomarkers with mechanistic interpretations of biological changes (associated with the gene signatures) appropriating for an implementation of therapeutic targeting.

### RELATED APPLICATIONS

The present application is related to U.S. patent application Ser. No. 13/255,898.

### FIELD OF THE INVENTION

The present invention relates to a method of identification of clinically and genetically distinct sub-groups of patients subject to a medical condition, particularly (but not exclusively) breast, lung, and colon cancer patients using a composition of respective gene expression values for certain gene pairs. It further relates to using respective gene expression values for these genes to predict patient risk groups (in context of patient survival or/and disease progression) and to using the predicted groups for identification of the specific and robust prognostic biomarkers with mechanistic interpretations of biological changes (associated with the gene signature) appropriate for an implementation of therapeutic targeting.

### BACKGROUND OF THE INVENTION

Breast cancer ranks second among commonly diagnosed cancers in the world and is the most frequent cause of cancer death in women in both developing and developed countries, although it is only the fifth greatest cause of cancer mortality overall [1]. During the last decade, substantial progress have been achieved in reducing the mortality of breast cancer (especially in developed countries) [1] as compared to its increasing incidence worldwide. The reasons for reduction of breast cancer mortality include application of early mammographic screenings [2] as well as adjuvant chemo-, hormono-therapy [3]. Nevertheless, the benefit of the adjuvant therapy and clinical outcome vary essentially among breast cancer patients [4]. For example, therapy modalities are often dramatically different depending on the tumor grade status (poorly differentiated tumors vs. highly differentiated tumors); targeted biologic therapy with trastuzumab or lapatinib is highly efficient in HER2/neu-positive breast tumors [5]. With the currently used post-surgery therapeutic treatments approaches about 60% of all breast cancer patients with early-stage breast cancer still receive adjuvant chemotherapy of which only a small proportion (2-15%) of patients derive therapeutic benefit [3]. All treated (and, often over-treated) patients (by systemic therapy) remain at risk of long-term toxic side effects which can include cognitive impairment, cardiac tissue damage, infertility, disease of the central nervous system, secondary malignancies and personality changes.

According to a recent report which included 29 US cost-of-illness studies for breast cancer, the estimate of lifetime per-patient costs of breast cancer ranges from $US 20,000 to $US 100,000 [6]. Costs of different surgeries are relatively similar (breast-conserving surgery vs. mastectomy) but, all else being equal, significant costs ($US 23,000-31,000) were observed for patients who received adjuvant chemotherapy compared with those who did not [6]. According to another source [7] the cost of breast cancer treatment for pre-invasive stages is approximately $US 10,000-$US 15,000, whereas by contrast later stage breast cancers (with higher grade, higher invasiveness and metastatic potential) can reach total cost of between $US 60 000 and $US 145 000. Therefore, improvement of the prognosis/prediction and further stratification of hormone therapeutic/chemo therapeutic schemes (which includes identification of patients with highly invasive/recurrent/metastatic tumors) could substantially improve life quality of individual patients and decrease per-patient treatment costs.

The relatively low efficiency of currently used chemotherapy schemes can be explained by the high level of heterogeneity of breast tumors, on the one hand, and by real challenges for its identification in routine everyday clinical practice, on the other. Nevertheless, very active research ongoing in the field nowadays including current report provides new opportunities and technological innovations to tackle those challenges.

Previous and very recent works reported a large number of parameters which are able to grasp breast cancer heterogeneity: clinico-pathological parameters, simple molecular biomarkers and complex clinical and multi-gene molecular classifiers (“gene signatures”). The first and second types of parameters include, for example, histological grade, estrogen receptor status, progesterone receptor status, lymph node status, Ki67 status, mitotic index, tumor size. The histological Nottingham Grading System discriminates 3 distinct grades: grade 1(G1), grade 2(G2) and grade 3(G3) [8]. NPI score is a typical example of a complex clinical biomarker which is based on three simple clinical parameters-tumor size, lymph node status and histological grade and can identify three prognostic groups with 10-year survival rates 83%, 52% and 13% [9]. However, Nottingham grading system has substantial limitations due to high genetic heterogeneity within each of subtypes. Not fully characterized genetic heterogeneity of G3, G2 and, most probably, G1 breast tumors could be one of the reasons of inconsistency in histologic grading between institutions and, as a consequence, the reason why some health institutions do not include histologic grading in their staging criteria [10, 11].

Intrinsic molecular classification independently sorted out all types of breast tumors into 5 distinct molecular subtypes different in prognosis and therapeutic treatment: basal-like, luminal A, luminal B, ERBB2-enriched and normal-like [12, 13]. Alternatively, in multiple recent studies application of novel complex multigene classifiers led to the discovery that some of the already classical intrinsic subtypes turned out to be heterogeneous in terms of survival [14, 15]. However, typically each of the classifiers was efficient only within one specific subtype and has limited tumor stratifying/prognostic power in the other subtypes.

Gene pairs as distinct prognostic biomarkers can have higher prognostic impact than individual genes in various cancers [16, 17]. The expression levels ratio (expression index) of two genes-HOXB13 and IL17BR—have been shown to be efficient in prediction of recurrence risk in ER-positive, lymph node negative breast cancer patients after hormonotherapy (tamoxifen) [17]. Nevertheless, a single-gene-pair ratio cannot cover all possible and obviously non-linear relationships between the genes and their associations with diseases, medical conditions and population variation. Mechanistic interpretation of the biological changes associated with the single gene ratio tests is not clear. Thus, such signatures have practical limitations in the context of sensitivity and specificity. The robustness of such single gene-pair classifiers for prognosis raised hot debates in the literature [18].

Below we determine several practical challenges in the process of making therapeutic decisions for cancer patients, and specifically breast cancer patients, which include:

i) making therapeutic decisions within poorly differentiated (G3 tumors) tumors, especially within basal-like G3 breast tumors, until now represents a problem for implementation by clinical oncologists;
ii) basal-like breast cancers representing 15-20% of invasive breast cancers are poorly differentiated high grade (typically, G2 or G3) tumors which frequently do not express hormone ER-, PgR- and ERBB2-receptors and are considered to have the worst prognosis [19]. This subtype is genetically more homogenous than the triple-negative group (i.e., ER“−”, PgR“−”, HER2“−”) [20], and therefore, problematic for clinical prognosis and optimal treatment.
iii) luminal A breast cancers which express hormone receptors, have an overall good prognosis and can be treated by hormone therapy, nevertheless even within this group it is necessary to identify tumors that will relapse and metastasize and might be treated with chemotherapy;
iv) grade 1 (G1) and grade 1-like breast tumors (G1, G1-like) are considered to be the low-risk prognosis group which can routinely be determined by histological analysis. However, within this group there is a substantial chance of relapse and metastasis cases which might be treated with chemotherapy;
v) Relatively “good” prognosis group of breast tumors predominantly includes ER-positive (ER“+”) and lymph node negative (LN“−”) patients. However, within that group, a subset of patients still develops tumor recurrence after curative surgery and adjuvant tamoxifen systemic therapy [21].

The biological functions and molecular processes of a significant number of genes in the computationally derived molecular signatures have not been well characterized in many of cancer sub-groups of interest (e.g. in G1 breast cancer), making the determination of the personalized diagnostics or prognosis genes unattainable. Additionally, functional interconnection of a collection of the genes in a signature (often derived computationally from the limited genome-wide studies) in a given cancer subtype is poorly understood. At present, identification of molecular targets for therapeutic intervention are only curiously considered in the computational strategies of the prognostic gene signature discovery methods.

Novel integrative computational, genome-wide and biological mechanism-driven strategies for cancers are promising to discover prognostic signatures that will provide oncologists with unbiased computational predictions and mechanistic interpretations of the pathobiology process associated with the identified gene signatures, enabling decision making about tumor subtype classification, disease recurrence risk stratification and the most appropriate therapeutic strategy of a patient. In particular, re-classification of the G2 breast cancer patients onto G1-like and G3-like subtypes identified to the 5-gene tumor aggressiveness gene (TAG) signature [22], in which genes are functionally associated to each other in a genome of breast cancer cells and play critical role within cell cycle, mitosis and kinetochore machineries. Only such an approach could permit an appropriate interpretation of the results and maximize the usefulness of the signature.

Sense-antisense gene pairs (SAGPs) are naturally occurring gene architectures in which paired genes are located on different strands of a chromosome, transcribed in opposite directions and share a common locus (overlapping region) [23] and, therefore, are functionally connected. Recent data indicate that the expressions of genes-members in SAGPs can be coordinated through specific molecular mechanisms which may not be applicable for the gene pairs without sense-antisense overlaps [24, 25, 26, 27, 28]. It has been shown that antisense transcription and alternative splicing are tightly coordinated processes [25, 27, 29, 30, 31]. Recently Morrissy et at [27] reported the role of SA overlapping regions on slowing down the PoIII complex and, as a consequence, increase of the alternative splicing rate at the same regions. Systematic changes/deregulation of co-expression profiles in such gene pairs have been shown to be directly or indirectly associated with pathogenesis of various cancers including breast, colon, lung, gastric and endometrial cancers as well as B-cell lymphomas and acute lymphoblastic leukemia [16, 23, 32, 33, 34]. Deregulation of co-expression profile in such gene pairs could be a driver of cancer progression and a source for discovery of novel and distinct molecular subtypes of breast cancer and other cancers. Specific and systematic changes of genes expression in cancer-relevant SAGPs could be systematically exploited to detect and to monitor the significant differences in tumor aggressiveness, to identify novel mechanically relevant and robust biomarkers for those differences and make prognosis/prediction of clinical outcome of cancer patients.

Thus, cancer-relevant SAGPs could be utilized to predict patient risk groups and subgroups (in context of survival time or/and disease progression) using respective gene expression values for these genes. The predicted groups could be further implemented for an identification of specific and robust prognostic biomarkers with mechanistic interpretations of biological changes (e.g., associated with the SAGPs signature) appropriating for therapeutic targeting.

Therefore, there is a continuing need in the art for systematic identification of cancer-relevant SAGPs coupled with their direct application in clinical practice.

### SUMMARY OF THE INVENTION

In general terms, the present invention proposes a computerized method of identifying candidate biomolecules relevant to a medical condition, the candidate biomolecules being putative clinical biomarkers for prognosis of, or putative therapeutic targets for treating, the medical condition. The method comprises identifying a set of SAGPs which optimally stratifies low-risk and high-risk patient sub-populations, identifying genes amongst the SAGPs which are differentially expressed between the sub-populations, and identifying biologically significant genes amongst the differentially expressed genes found in the patient sub-populations The SAGPs may be those listed in Tables 1A and 1B, for example, which are cis-anti-sense interconnected gene pairs.

The invention also provides methods and kits for prognosis of survival or/and treatment response, for example using the identified differentially significant genes belonging specific biological mechanisms. Embodiments of the invention provide a computational method for identification of SAGPs which are relevant to a variation of medical condition and disease outcome, particularly breast cancer. Embodiments also provide an implementation of this method providing identification of statistically and biologically specific patient stratification and prognostic disease models via the cancer relevant small gene signatures (prognostic predictors). Such strategy allows a mechanistic interpretation of pathobiological changes in the tumors and their subtypes associated with the deducted prognostic molecular signatures for patient stratification and prognosis, and for identification of appropriate prognostic biomarkers for the most optimal therapeutic intervention.

In one aspect, the present invention provides a computerized method of identifying candidate biomolecules relevant to a medical condition, the candidate biomolecules being putative clinical biomarkers for prognosis of, or putative therapeutic targets for treating, the medical condition, the method comprising:
for each subject k of a set of K subjects suffering from the medical condition, receiving subject data which indicates (i) for each gene pair i, j of a plurality of sense-antisense gene pairs (SAGPs), corresponding gene expression values y_{i,k}, y_{j,k} of subject k; and (ii) a survival time and survival event of subject k;
identifying, using said subject data, a prognostic subset of said SAGPs which optimally stratifies the subjects into low-risk and high-risk disease progression subgroups;
comparing gene expression values of each gene in the low-risk and high-risk subgroups which have been stratified by said prognostic subset of SAGPs, to identify a set of prognostic genes which are differentially expressed between the low-risk and high-risk subgroups; and
identifying one or more predefined biologically-related categories of genes which are over-represented in the set of differentially expressed prognostic genes, wherein the candidate biomolecules comprise genes or gene products belonging to said over-represented categories.

In another aspect, the present invention provides a computerized method of clinical outcome prognosis in a subject having a medical condition, the method comprising:
receiving data representing parameters of one or more statistical partition models (SPMs) said SPMs being configured to stratify a cohort of subjects having the medical condition into subgroups, said parameters representing, for each gene pair of one or more sense-antisense gene pairs (SAGPs), a pair of lines in a two-dimensional space spanned by respective expression level values of respective genes i, j in the gene pair, the pair of lines being formed using two cut-off values cⁱ and c^{j}, and each of the lines having a non-zero angle α to each of two axis directions in the space indicating increasing values of a corresponding one of the expression level values;
receiving expression level data representing expression levels in the subject of genes of one or more selected SAGPs; and
for each SAGP of the selected SAGPs, comparing the expression levels to the pair of lines for the SAGP to obtain comparison data indicating on which side of the pair of lines the expression values for the subject lie, thereby obtaining a prediction of a subgroup to which the subject belongs.

In a further aspect, the present invention provides a kit for predicting clinical outcome in a subject having a medical condition, the kit comprising: a plurality of polynucleotide sequences, ones of the plurality of polynucleotide sequences being capable of specifically hybridizing to and/or detecting a gene of a plurality of genes and/or an expression product of the gene to obtain respective gene expression values, wherein the plurality of genes comprises one or more of the sense-antisense gene pairs (SAGPs) listed in Table 1A, and written instructions for comparing, and/or a tangible computer-readable medium having stored thereon machine-readable instructions for causing a computer processor to compare, the respective gene expression values to optimal gene expression cut-off values, wherein the plurality of genes comprises no more than 100 genes; and wherein the optimal gene expression cut-off values are determined for each SAGP by:
(i) defining a plurality of trial values for each of two cut-off values cⁱ and c^{j};
(ii) for each of a plurality of angles α, for each subject, and for each of the trial cut-off values cⁱ and c^{j};
(a) comparing the expression values to a respective pair of lines in a two-dimensional space spanned by the expression values to obtain comparison data indicating on which side of the pair of lines the expression values for the corresponding subject lie, the pair of lines being formed using the cut-off values cⁱ and c^{j}, each of the lines having angle α to a direction in the space indicating increasing values of a corresponding one of the expression values; and
(b) generating at least one SPM based on the comparison data; and
(iii) selecting the one of the SPMs (‘the maximally predictive SPM’) which has the maximal statistical value in predicting the survival times of the subjects,
whereby the cut-off values cⁱ and c^{j} for the maximally predictive SPM are the optimal gene expression cut-off values.

In a yet further aspect, the invention provides a computerized method of composite survival prediction combining the output values from a plurality of SPMs associated with prognosis of a potentially fatal medical condition in each subject k of a set of K subjects suffering from the medical condition, each SPM being a model of the statistical significance of the expression level values of a corresponding set of one or more genes or gene pairs, the method employing test data which for each gene i of the pair of genes indicates a corresponding gene expression value y_{i,k} of subject k;
the method including:
for each subject obtaining for each of the SPMs a respective risk level value indicative of a risk level for the subject;
forming a weighted average of the risk level values using a set of respective weights, the weights being indicative of the relative quality of patient separation according to the given SPM versus others of the respective models in context of statistical significance of the relative risk statistics of the medical condition;
comparing the weighted average with a cut-off value to obtain a prognosis value.

In a still further aspect of the present invention, there is provided a method of prognosis of survival or treatment response in a subject suffering from breast cancer, comprising:

obtaining a test sample from the subject;

measuring a gene expression level in the test sample for one or more of the prognostic genes obtained according to the first or second aspects of the invention and listed in Table 11; and
comparing the measured gene expression level to a predefined threshold;
wherein a measured gene expression level which is above the predefined threshold is indicative of a poor prognosis.

In a still further aspect, the present invention provides a kit for prognosis of survival or treatment response in a subject having breast cancer, the kit comprising: at least one nucleic acid probe capable of specifically hybridizing to and/or detecting a gene of a plurality of genes and/or an expression product of the gene, wherein the plurality of genes comprises one or more of the genes listed in Table 11, and wherein the plurality of genes comprises no more than 200 genes.

In yet another aspect of the present invention, there is provided a system for identifying candidate biomolecules relevant to a medical condition, the candidate biomolecules being putative clinical biomarkers for prognosis of, or putative therapeutic targets for treating, the medical condition, the system comprising at least one processor and a tangible computer-readable storage medium having stored thereon machine-readable instructions which, when executed, cause the at least one processor to:
for each subject k of a set of K subjects suffering from the medical condition, receive subject data which indicates (i) for each gene pair i, j of a plurality of sense-antisense gene pairs (SAGPs), corresponding gene expression values y_{i,k}, y_{j,k} of subject k; and (ii) a survival time and survival event of subject k;
identify, using said subject data, a prognostic subset of said SAGPs which optimally stratifies the subjects into low-risk and high-risk disease progression subgroups;
compare gene expression values of each gene in the low-risk and high-risk subgroups which have been stratified by said prognostic subset of SAGPs, to identify a set of prognostic genes which are differentially expressed between the low-risk and high-risk subgroups; and
identify one or more predefined biologically-related categories of genes which are over-represented in the set of differentially expressed prognostic genes, wherein the candidate biomolecules comprise genes or gene products belonging to said over-represented categories.

The method may include genome wide screening and selection of a relatively large number (at least 50 SAGPs) to identify SAGPs which are significantly correlated with the medical condition and survival disease outcome data, and then use them to construct a statistics-based prognostic algorithm/method which can generate a most predictive statistical partition model (SPM) based on the estimated cut-offs of gene expression values of the SAGPs. The SAGP for which their best SPM is found is then used for construction of the composite prognosis model (CPM) and stratification of the patients according to the estimated risk outcome.

Next, the method may use the patient classification provided by SAGP CPM for further identification of the specific and reliable differentially expressed genes (DEG) signature in context of discovery of mechanistically related biomarkers (e.g., spliceosome prognostic gene signature) including the genes which could be the most appropriate for therapeutic targeting.

In one embodiment, a method referred to herein as 2-Dimensional Rotated Data-Driven grouping (“2D RDDg”) is provided. In 2D RDDg, expression level values for two genes of a gene pair, expressed as points in a two-dimensional space spanned by the expression level values of a plurality of subjects, are compared to perpendicular cut-off lines which are iteratively rotated in the two dimensional space at a succession of incrementally different angles, performing stratification of the subjects into two subgroups (e.g. low- and high-risk) during each iteration, without losing their orthogonality property, to improve the quality of a statistical partition/dichotomization model in relation to a medical condition or a genetic or phenotypic variation.

In other embodiments, there is provided a computer-implemented method for identification of prognostic SAGPs, comprising: receiving expression data indicative of expression levels of a plurality of genes of a plurality of sense-antisense gene pairs (SAGPs) for a plurality of subjects; identifying, from the expression data, SAGPs for which expression levels of genes in respective pairs are significantly correlated with each other and with a survival or treatment outcome for a medical condition; and identifying a set of prognostically significant SAGPs from among the identified SAGPs using 2D DDg or 2D RDDg. Each of the prognostically significant SAGPs assigns (stratifies) each subject to a low- or high-disease development risk subgroup, refined by the 2D DDg or 2D RDDg method. The method may further comprise applying a weighted voting procedure to p-values of the prognostically significant SAGPs to the stratified subjects to obtain a weighted voting grouping for each subject.

Embodiments of the invention make it possible to extract SAGPs relevant to a medical condition such as cancer, or breast cancer, as well as their combinations which are highly prognostically significant within the diverse subgroups/subtypes of the medical condition.

A computational algorithm (2D RDDg) for patient grouping may be specifically adapted for the usage of those SAGPs and substantially improves the accuracy of stratification and prognosis of patients' outcome. Embodiments of the invention make it possible to substantially improve the accuracy of classification of any pathological samples using survival analysis.

Embodiments of the present invention also propose a sense-antisense gene classifier SAGC as a complex biomarker as a specific subset of gene pairs to substantially improve the accuracy of classification of breast cancer tumors into low risk (LR) and high risk (HR) subgroups. This classifier either outperforms or has a comparable accuracy of stratification and clinical outcome prognosis as compared with currently known complex multi-gene biomarkers/classifiers and clinical tests/assays.

Specifically, embodiments of the present invention propose a new molecular classifier: a sense-antisense gene classifier (SAGC) which is composed of 12 distinct classification units—sense-antisense gene pairs (SAGPs) or 24 individual genes, correspondingly.

These gene pairs are shown in Table 1B below.

The molecular classifier can be used for stratification and prognosis/prediction of novel LR and HR subgroups within total unselected groups as well as within various characterized subgroups/subtypes of breast cancer. The classifier is demonstrated below to be of use for nine different subgroups/subtypes of breast tumors and for tumors of two other epithelial cancers: ER“+”, LN“−” breast tumors treated with tamoxifen; ER“+”, LN“−” PgR“+” breast tumors with size not exceeding 2 cm before curative surgery and not received systemic treatment; grade 3 (G3) breast tumors; G3 and G3-like breast tumors; G1 and G1-like breast tumors; G1 breast tumors; ER“−” breast tumors; basal-like grade 3 breast tumors and luminal A breast tumors, colon cancer stage II tumors and non-small lung cancer tumors. The proposed SAGC classifier substantially outperforms many of the currently known classifiers in accuracy. At the same time, the same set of gene pairs (and a multigene assay) can be used for various molecularly distinct subpopulations of breast tumors, which is not possible for any of the currently known classifiers. Therefore, the SAGC classifier is, to our knowledge, the first multitask complex multi-gene classifier of breast cancer ever proposed based on gene expression studies. We further expect that the classifier could be highly efficient in other subpopulations of breast tumors.

Typically, the classifier contains a core sense-antisense gene pair for a specific subpopulation of breast cancer under prognosis: for example, the SAGP (RNF139/TATDN1) for ER“+”, LN“−” breast cancer patients shows similar accuracy in prognosis of clinical outcome as the currently commercially available two-gene classifier HOXB13/IL17BR. In order to improve the accuracy of our classifier in each of the specific breast tumors subpopulations, additional gene pairs could be introduced in the classifier (maximum number of additional gene pairs-11).

In the era of stratified and personalized medicine a cancer patient with a tumor categorized into a subpopulation or subtype of tumors distinct in terms of molecular etiology and/or patient survival would receive a distinct stratified/individual treatment scheme. This can optimize the ratio: treatment efficiency/life quality for each individual patient. In that context the routine and accurate identification of novel molecular subgroups within the known clinical/genetic subgroups and subtypes would be very helpful to achieve that important goal.

### BRIEF DESCRIPTION OF THE FIGURES

Embodiments of the invention will now be described, by way of non-limiting example only, with reference to the accompanying figures, in which:

FIG. 1 is a flow diagram showing the derivation of a classifier in a method which is an embodiment of the invention;

FIG. 2 is a diagram describing the usage of the classifier;

FIG. 3 illustrates the principle of partition of tumors/patients using 2-D DDg survival analysis as an example of implication of a statistical partition model;

FIG. 4 shows experimental data demonstrating the superiority of the 2-D RDDg method over the 2-D DDg method used in the embodiment of FIG. 1;

FIG. 5, which is composed of FIGS. 5(a) and 5(b), illustrates the synergistic effect on patient survival for two SAGPs from the SAGC classifier as compared with patient survival for individual genes of the same SAGPs;

FIG. 6, which is composed of FIGS. 6(a)-(c), illustrates the prediction of clinical outcome and stratification for ER-positive, LN-negative breast cancer patients who received systemic tamoxifen treatment as well as for ER-positive, LN-negative and PgR-positive breast cancer patients who did not receive any systemic treatment, using the SAGC classifier;

FIG. 7 illustrates the prognosis of clinical outcome and stratification for grade three breast cancer patients using the SAGC classifier;

FIG. 8 illustrates the prognosis of clinical outcome and stratification for grade three and grade three-like breast cancer patients using the SAGC classifier;

FIG. 9 illustrates the prognosis of clinical outcome and stratification for grade one and grade one-like breast cancer patients using the SAGC classifier;

FIG. 10 illustrates the prognosis of clinical outcome and stratification for grade one breast cancer patients using the SAGC classifier;

FIG. 11 illustrates the prognosis of clinical outcome and stratification for ER-breast cancer patients using the SAGC classifier;

FIG. 12 illustrates the prognosis of clinical outcome and stratification for breast cancer patients with basal-like G3 tumors using the SAGC classifier;

FIG. 13 illustrates the prognosis of clinical outcome and stratification for breast cancer patients with Luminal A tumors using the SAGC classifier;

FIG. 14, which is composed of FIGS. 14A and 14B, illustrates the prognosis of clinical outcome and stratification for A) colon cancer patients with stage II tumors, B) patients with non-small lung cancer, using the SAGC classifier;

FIG. 15, which is composed of FIGS. 15A to 15G, illustrates the higher accuracy and robustness of the full SAGC in stratification of breast tumors as compared with distinct SAGPs;

FIG. 16, which is composed of FIG. 16A-16G, illustrates partitions of breast cancer patients in 5 unselected total groups. A and B are the Uppsala and Stockholm cohorts (training groups); and C, D, E, F and G are the Marseille, Harvard, Origene, Singapore and Metadata cohorts correspondingly (testing groups);

FIG. 17, which is composed of FIG. 17A-17J, shows characteristics of breast cancer patients belonging to the HR subgroups identified by the SAGC from total unselected groups as well as novel potential genes-biomarkers/drug targets candidates—for HR subgroups derived when applying SAGC.

FIG. 18 illustrates the principle of iterative rotation of X- and Y-axes in the 2-D RDDg method as an improvement of the 2-D DDg method for patient partitioning where X- and Y-axes have been fixed and only a limited number of design combinations (14) were possible.

FIG. 19, which is composed of FIGS. 19A and 19B, illustrates comparisons of the set of SAGC-associated genes with the set of genes of Genetic Grade Signature and with the set of breast cancer-associated genes derived from the MalaCard database.

FIG. 20, which is composed of FIGS. 20A and 20B, illustrates partitions of 42 unselected breast cancer patients in which technical validation of SAGC was performed. FIG. 20A shows partitioning using nine SAGPs of SAGC (Table 9) as applied using 2D RDDg and WVG procedures (training mode) to microarray expression data; FIG. 20B shows partitioning using the same nine SAGPs of SAGC (Table 9) as applied using 2D RDDg and WVG procedures (training mode) to QRT-PCR expression data; and

FIG. 21 is a block diagram of an exemplary system for implementing methods according to embodiments of the invention.

### DEFINITIONS

As used herein, gene expression level value is a measure of expression activity of a gene by detection of mRNA and for the protein molecules in a given tissue sample.

As used herein, a combination refers to any association between or among two or more components. The combination can be two or more separate components, such as two compositions or two collections, can be a mixture thereof, such as a single mixture of the two or more items, or any variation thereof. The items of a combination are generally functionally associated or related.

As used herein, the term “comprising” is to be interpreted as specifying the presence of the stated features, integers, steps or components as referred to, but does not preclude the presence or addition of one or more features, integers, steps or components, or groups thereof. However, in context with the present disclosure, the term “comprising” also includes “consisting of”. The variations of the word “comprising”, such as “comprise” and “comprises”, have correspondingly varied meanings.

The term “gene pair” refers to a combination of two selected nucleic acid sequences. The two selected nucleic acid sequences can be two separate components, such as two compositions. For example, the two selected nucleic acid sequences may be immobilized at two discrete positions on a solid substrate. Correspondingly, a combination of gene pairs refers to at least two such gene pairs (i.e. at least four selected nucleic acid sequences). With a combination of two or more gene pairs, each selected nucleic acid sequence may be immobilized at discrete positions forming an array on a solid substrate.

The term “risk”, or “relative risk” refers to a measure of separability between two (or more) Kaplan-Meier survival curves related to the potentially fatal medical condition or disease.

The term “statistical partition model (SPM)” defines cut-off values of gene expression level values (low or high) and typically also other necessary parameters (e.g., partition design, rotation angle (see Methods) for a gene or a gene pair in a given group of tumor samples (obtained from distinct patients) and stratifies them into subgroups with, respectively, a relatively high-risk- and a low-risk of a potentially fatal medical condition.

The term “medical condition associated feature” refers to any gene product (e.g. mRNA, (gene expression values detectable by micro-array, PCR-based assays, or other mRNA quantification techniques such as massively parallel sequencing) or protein (detected by immuno-staining, mass-spectrometry, etc) or any other quantitative features (e.g. clinical classification score) useful for discrimination between different states or degrees of a medical condition, and may include combinations of such features (e.g. a ratio of the RNA expression levels, produced by a given gene set, expressed in the same tissue or tissues of a given a patient).

The term “prognostic method”, as used herein, refers to a stratification of patients with a medical condition (e.g. cancer) into two (or more) survival significant sub-groups via any “process of optimization”, including (but not limited to) (i) a rank-order of the patients with a given medical condition according a medical condition associated feature value (e.g., gene expression value) of a training data set and (ii) an identification of cut-off value(s), splitting this feature value onto two (or more) grades which via a survival prediction model (e.g., Data Driven grouping (DDg)) assign the patients with such medical condition to one of statistically distinct disease development risk sub-groups.

The method of “composite survival prediction” (CSP) refers to the group of prognostic methods which integrates the information for individual features (e.g., genes or gene pairs expression signals) into a significantly improved integrated partition of the patients. CSP includes, but is not limited to, Weighted Voting Grouping (WVG), Hierarchical Clustering Analysis (HCA) and Principal Component Analysis (PCA).

The term “disease prognosis model” (DPM) refers to a mathematical model of optimization procedure of the patient stratification into low-risk and high-risk subgroups implemented through the use of any of SPMs and any of methods of CSP. For a given patient, DPM with the most appropriate SPMs and CSP (optimized using training dataset(s)) is used for prognosis/prediction of patient “relative risk” and/or clinical outcome.

As used herein, “differentially expressed” means that a gene is expressed differently, for example in mRNA level, in two or more given samples or groups of samples. The gene may be determined to be differentially expressed by any method known in the art, for example by applying a fold-change threshold for the relative expression level or relative mean expression level in the two samples, or by a parametric or non-parametric statistical testing procedure such as a t-test (including a moderated t-test such as that disclosed in [35]), or for digital gene expression measurement platforms such as mRNA-Seq, Fisher's exact test or likelihood ratio statistics based on a generalized linear model (see, for example, Bullard, J. H. et al, [36] and references cited therein).

The term “original/total group of BC patients” refers to the entire cohort of patients from a given clinical center or hospital without any preselecting by clinical and pathological parameters or conventional clinical biomarker (e.g., ER-status, Histological grade, Ki67 etc.).

The term “Functional gene annotation/Gene Ontology” refers to the bioinformatics project providing ontology of defined terms representing genes and their product properties and covering three gene ontology classes: cellular component, molecular function and biological process.

Functional Gene Annotation/Gene Ontology Enrichment Analysis (FGA/GO EA) is refers to an estimation procedure whether certain Functional Gene annotation/Gene Ontology categories or terms in a gene list are present in higher numbers than it would be expected by chance using a statistical test as known in the art (e.g., Fisher's exact test, or a hypergeometric test, with p-values adjusted using a multiple-testing correction method such as the Holm-Bonferroni method, or a method of controlling the false discovery rate, such as the Benjamini-Hochberg procedure).

The term “polynucleotide sequence” refers to a sequence of nucleotides in a biopolymer composed of 13 or more nucleotide monomers covalently bonded in a chain.

As used herein, the term “oligonucleotide” refers to a short single-stranded nucleic acid biopolymer (typically from 2 to 100 bases) composed of nucleotides and used for artificial gene synthesis, DNA sequencing, as molecular hybridization probes at discrete positions on a solid substrate, and for polymerase chain reaction (PCR).

The term “oligonucleotide sequence” refers to a sequence of nucleotides in an oligonucleotide.

Accordingly, an array refers to a plurality of biological molecules (e.g., oligonucleotides, polypeptides, antibodies, etc) immobilized at discrete positions on a solid substrate. Typically, the position of each of the molecule in the array is known, so as to allow for identification of a target molecule in a sample following analysis.

As used herein, the term “microarray” refers to a substrate comprising a plurality of biological macromolecules (e.g., proteins, polypeptides, nucleic acids, antibodies, etc.) affixed to its surface. In some embodiments, the location of each of the macromolecules in the microarray is known, so as to allow for identification of the samples following analysis.

The term “DNA microarray” refers to a solid support platform (nylon membrane, glass or plastic) on which single stranded DNA is printed or otherwise affixed (for example, as part of a masked or maskless photolithographic fabrication process) in localized features (e.g. nucleic acid probes or probesets for detecting gene expression) that are arranged in a regular grid-like pattern.

The term “reverse transcription polymerase chain reaction” refers to the method used to quantitatively detect gene expression though creation of complimentary DNA from transcribed RNA.

### DETAILED DESCRIPTION OF THE EMBODIMENTS

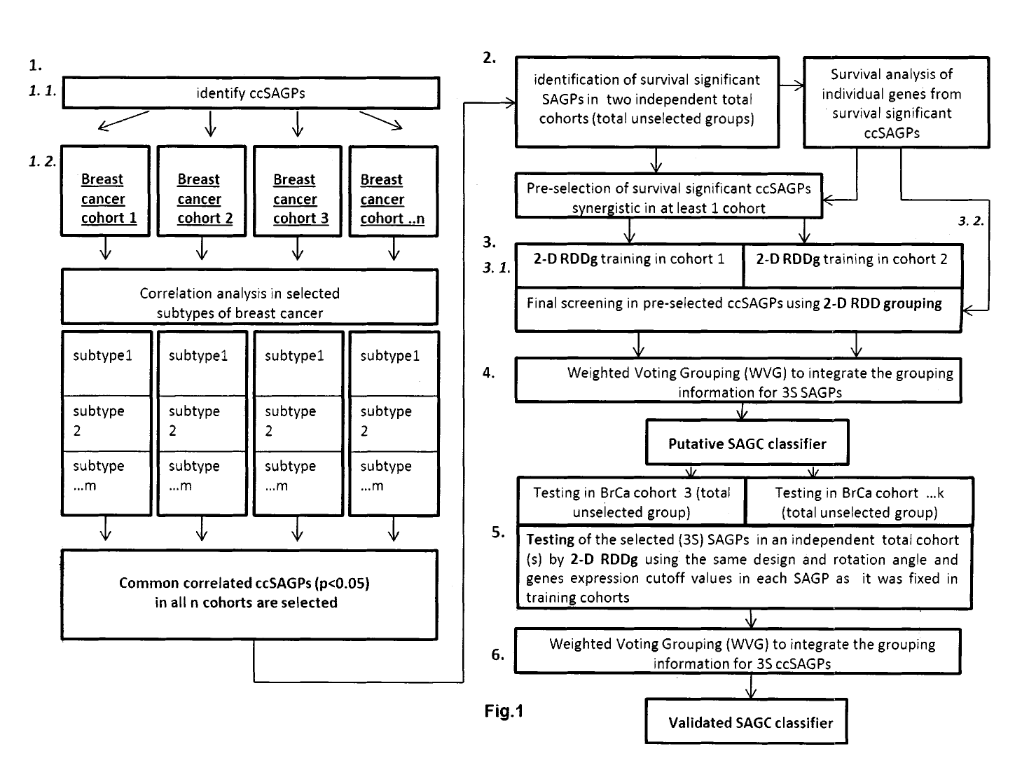

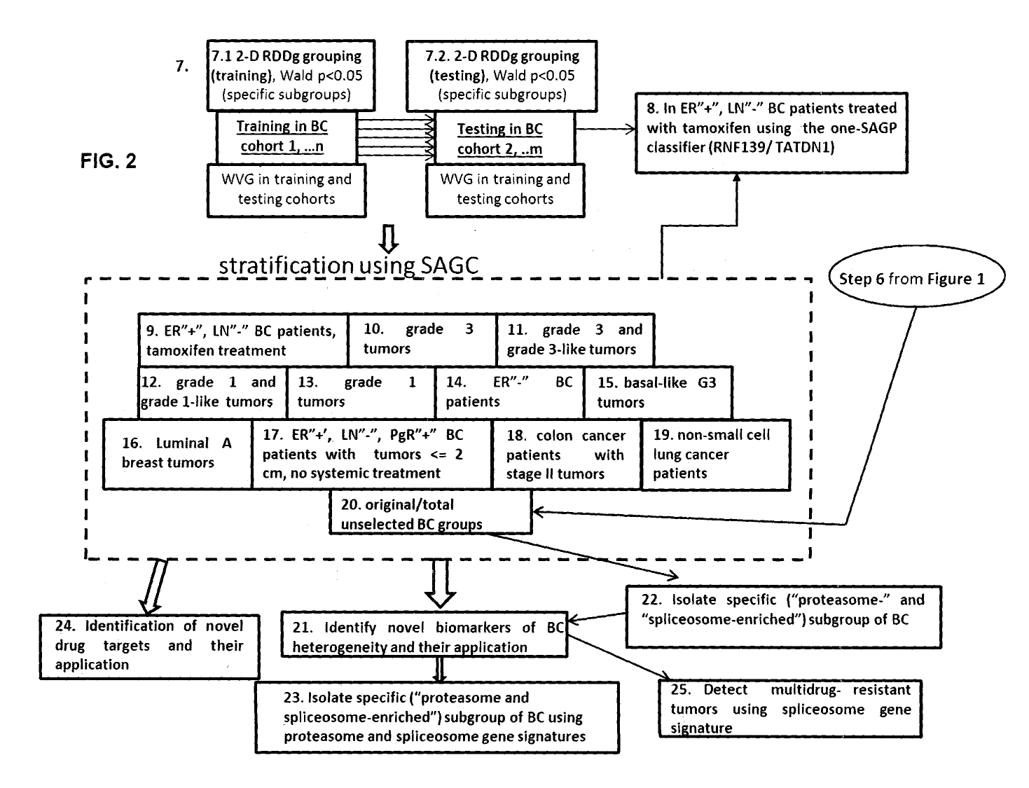

FIG. 1 shows the steps of a computational method for generating a SAGC classifier according to embodiments of the invention. The steps are explained below, and we simultaneously explain an example which implements the steps.

Herein, we deal with but one essential subclass of SAGPs in which each gene-partner can encode a protein (coding-coding SAGPs-ccSAGPs). The genes of ccSAGPs are highly populated in the genome, relatively higher expressed in cancer cells and better annotated than other classes of SAGPs (non-coding-coding or non-coding-non-coding SAGPs). Besides, in ccSAGPs expression patterns of both genes-partners could be mutually regulated effecting the levels of their protein products with presumably stronger combined impact for the cells fate.

A first step (step 1 in FIG. 1) is the isolation of ccSAGPs relevant to a medical condition, such as cancer or breast cancer. Based on public literature analysis and our own previous studies, we suggested that ccSAGPs in which gene partners show significant correlations of their expression values across samples can have functional and/or clinical relevance to a medical condition, such as cancer or breast cancer. The method for isolation of breast cancer-relevant ccSAGPs (BCR-ccSAGPs, or hereafter BCR-SAGPs) described below is applicable to any sense-antisense transcript pairs and any sense-antisense gene pairs. This is performed by the following sub-steps of step 1:

Step 1.1. All ccSAGPs from publicly available annotation databases (e.g., USAGP database [29]) are identified by (manually and/or automatically) searching the databases;
Step 1.2. Gene pairs identified in step 1.1 are screened to select BCR-SAGPs. This step may be done using the criteria of significant Kendall tau correlations (p<0.05) which assumes that if gene expression levels for genes in a sense-antisense gene pair are significantly correlated across patients they could be co-regulated by common biological/molecular mechanism(s). This step is done in at least three independent cohorts to guarantee the robustness of the selected gene set. Selection of ccSAGPs with significant correlations is done within already characterized subgroups and subtypes (e.g., grade 3 tumors, basal-like subtype or grade 3 tumors, non-basal-like subtypes) of breast tumors in order to minimize effect of false-positive correlations and the fraction of less relevant gene pairs. Correlation analysis is performed for each cohort and each subgroup, to produce a respective set of ccSAGPs with significant correlations between the genes-partners included in each ccSAGP and finds those ccSAGPs which are in common subset found across the cohorts.

In one example, we selected the robust set of 73 BCR-SAGPs (Table 1A) within the groups of patients with Grade 3 tumors of basal-like subtype and within the combined groups of patients with Grade 3 tumors of “non-basal-like” subtypes (ERBB2-enriched+Luminal A+Luminal B+Normal-like subtypes) from 3 independent breast cancer cohorts (Uppsala, Stockholm and Harvard 1).

Steps 2-6. Screening and validation of gene pairs to select synergistic survival significant ccSAGPs (referred to herein as 3S-SAGPs). This may be done using the criteria of survival significance (Wald p<0.05).

Step 2 is to perform survival analysis of the ccSAGPs obtained in step 1. The survival analysis procedure we developed for this proposal is performed for pre-selection of synergistic survival significant ccSAGPs and uses a combination of 1D-DDg and 2-D DDg procedures. The 2-D DDg method is used to pre-select survival significant ccSAGPs; within the pre-selected ccSAGPs, and the 1D-DDg method is used to select 3S-SAGPs.

The 2-D DDg method is itself an extension of an algorithm known as the one-dimensional (1-D) DDg method [37]. The 1-D DDg method associates clinical data to single gene expression data, available for a set of patients K suffering from a medical condition, via survival analysis with the Cox proportional hazards model. We denote the clinical and gene expression data for each patient k=1, K as (tₖ, eₖ, y_{i,k}) where tₖ indicates the survival time, eₖ is a binary outcome of patient's k status at time tₖ (e.g. eₖ=1 if relapse occurs and 0 otherwise) and y_{i,k} is the expression value of gene i, i=1, . . . , N. The 1-D DDg method finds for each gene i an optimal cut-off value cⁱ, that partitions the K* subjects into those with expression values (or log transformed expression values) above and below the threshold. The 1-D DDg tries out a number of trial values for cⁱ, and for each trial value, it finds the subset of the K subjects such that y_{i,k} is above the trial value of cⁱ. The survival times/events are fitted to a Cox proportional hazard regression model,

log hₖⁱ(tₖ|xₖⁱ,βᵢ)=αᵢ(tₖ)+βᵢxₖⁱ (1)

using a regression parameter βᵢ corresponding to the gene i, and then the regression parameter βᵢ is used to obtain a Wald p-value (significance value) indicative of the prognostic significance of the gene, using

$$P value ( {β}_{i} ) = Pr ( \frac{{β}_{i}^{2}}{var ( {β}_{i} )} > {χ}_{v}^{2} )$$

where χᵥ² denotes the chi-square distribution with v degrees of freedom. The algorithm then finds the trial value of cⁱ such that this significance value is maximized. This gives the cut-off value cⁱ for which gene i has maximal prognostic significance. The algorithm can then estimate which genes are associated with the medical condition: the ones for which the maximum prognostic significance is highest.

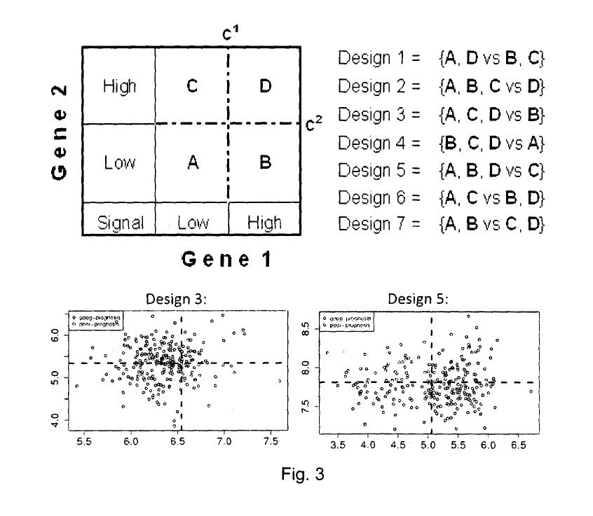

The 2-D DDg method [37] extends this idea to gene pairs, assuming that in some situations the expression values of individual genes organized in 2-dimensional space as gene pairs may provide a better statistical partition model of survival prognosis than the expression levels of individual genes organized in 1-dimensional space. A pair of genes is labeled i,j. The method uses a number of “designs” (models) illustrated in FIG. 3, which shows a two dimensional plot with yᵢ, yⱼ, as axes. The 2-D area is divided into four regions A, B, C and D, defined as follows:

A: y_{i,k}<cⁱ and y_{j,k}<c^{j}

B: y_{i,k}≧cⁱ and y_{j,k}<c^{j}

C: y_{i,k}<cⁱ and y_{j,k}≧c^{j}

D: y_{i,k}≧cⁱ and y_{j,k}≧c^{j} (2)

Each of the seven models is then defined as a respective selection from among the four regions:

Design 1 indicates whether the subject's expression signal are within regions A or D, rather than B or C.

Design 2 indicates whether the subject's expression levels are within regions A, B or C, rather than D.

Design 3 indicates whether the subject's expression levels are within regions A, C or D, rather than B.

Design 4 indicates whether the subject's expression levels are within regions B, C or D, rather than A.

Design 5 indicates whether the subject's expression levels are within regions A, B or D, rather than C.

Design 6 indicates whether the subject's expression levels are within regions A or C, rather than B or D.

Design 7 indicates whether the subject's expression levels are within regions A or B, rather than C or D.

Note that design 6 is equivalent to asking only whether the expression level of gene 1 in the subject is below or above c¹ (i.e. it assumes that the expression value of gene 2 is not important). Model 7 is equivalent to asking only whether the expression for gene 2 in the subject is above or below c² (it assumes that the expression value of gene 1 is not important). Thus, models 1-5 are referred to as “synergetic” (1-5), and the models 6 and 7 as “independent”.

The 2-D DDg algorithm considers all pairs of genes (i, j) in turn. For each pair, it considers each of the seven designs. For each design, it obtains a unique patients' grouping. For example, for design 1, the following subjects' grouping is obtained: patients with expressions (y_{i,k}, y_{j,k}) falling in A and D belong to Group 1; patients with expressions (y_{i,k′}, y_{j,k′}) falling in B and C belong to Group 2. Thus in Group 1 are the subjects with y_{i,k}<cⁱ and y_{j,k}<c^{j} or yi,k≧c^{j} and yj,k≧c^{j}. Let us define a parameter x_{i,j,k}^{m}, where x_{i,j,k}^{m}=1 if and only if, for genes i and j, and design m (m=1, . . . 7), the expression levels y_{i,k} and y_{j,k} meet the conditions of design m. The algorithm then fits the survival values to the Cox proportional model:

log h^{i,j}ₖ(tₖ|x_{i,j,k}^{m},β_{i,j}^{m})=α_{i,j}(tₖ)+β_{i,j}^{m}·x_{i,j,k}^{m}, (3)

and finds the design with the smallest Wald p-value β_{i,j}^{m} (i.e. highest statistical significance). The algorithm then seeks the pairs of genes for which this significance value is the smallest. Thus the algorithm has found both a significant pair of genes, and a design indicating which form of correlation between the genes' expression levels is statistically significant to the medical condition.

Note that FIG. 3 is based on the horizontal and vertical axes X and Y, each of them indicating a direction in which the expression level of only a single gene increases.

Step 3 is performed in order to select the highly robust synergistic survival significant ccSAGPs and utilizes another survival analysis procedure which is an extension of the 2-D DDg method [37], adapted to any correlated gene pairs (including ccSAGPs and other subclasses of sense-antisense transcripts and gene pairs). The extension is termed “2-D Rotated Data-Driven grouping” (2-D RDDg).

The rotated 2-D Data-Driven grouping (2-D RDDg) is a generalization of the 2-D DDg algorithm that considers patients' grouping using different angles for separating the data. In other words, the original X, Y axes are iteratively rotated by angle α, without losing their orthogonality property, and in each rotation the patients are grouped as before. The best grouping is the one that minimizes the Wald P value of the β coefficient of the Cox proportional model.

Note that instead of rotating (transforming) the data by using trigonometric functions:

$$[ \begin{matrix}{X}^{′} \\ {Y}^{′}\end{matrix} ] = [ \begin{matrix}X \\ Y\end{matrix} ] \times [ \begin{matrix}cos ( α ) & - sin ( α ) \\ sin ( α ) & cos ( α )\end{matrix} ]$$

where X′, Y′ and X, Y denote the new and the old coordinates, respectively, the algorithm is preferably implemented by rotating the axes themselves. In fact, these two possibilities are equivalent mathematically, but it is conceptually easier for a viewer to see different grouping patterns when the axes are rotated.

The steps of an implementation of the 2-D RDDg algorithm are as follows. Assume that, for each of a number of subjects k=1, K, expression level data exists for each of n gene pairs, where n is at least 10, or much higher.

1. A pair of genes is generated, and considered as a probeset pair denoted by i,j where i takes values in the range 1, . . . , N−1, and j takes values in the range i+1, . . . , N. For each probeset of the pair, form the candidate cutoffs vectors \vec{w}ⁱ=yᵢ* and \vec{w}ⁱ=yᵢ* of dimension 1×Q each, where Q is an integer. The values of \vec{w}ⁱ are expression levels for gene i falling into (q₁₀ⁱ, q₉₀ⁱ), i.e. the range of values between the 10^{th} and 90^{th} quantiles of the distribution of the log-transformed intensities. Similar logic holds for \vec{w}^{j}. We generate all Q² trial cut-off pair values of the predefined quantiles. Thus, each element of the (\vec{w}ⁱ, \vec{w}^{j}) pair is a trial cutoff pair value for gene pair i, j.

For 1-D DDg, the value of Q depends on the sample size. In the Stockholm cohort we have 159 samples (patients) and within the (q₁₀ⁱ, q₉₀ⁱ) interval there are approximately Q=120 patients. In the Uppsala cohort, Q is approximately 220.

For 2-D DDg, we need all possible pairs, so in the Stockholm cohort Q=120*120 (all 120 values of gene i for all 120 values of gene j) and in the Uppsala Q=220*220 (similarly). So, there is no standard Q value. It is determined from the data. The standard values for this algorithm are that we always take the 10^{th} and 90^{th} quantiles of the distribution of the expression levels.

Optionally, a “filtration step” is performed in which the algorithm finds which of the Q trial cut-off values in produces the global minimum P value in a 1-D DDg algorithm (i.e. each trial cut-off value is used to partition the patients, and the result is fitted to Eqn. (1)), and a number (e.g. 10) of other trial cut-off values having the next lowest P values. Then, the Q-dimensional vector of cut-offs for gene i is replaced by a vector having only these cut-off values. The filtration can do the same for \vec{w}^{j}. Subsequently, only the “filtered” cut-off pairs are considered in the 2-D version of the algorithm.

2. Denote each element of \vec{w}ⁱ as \vec{w}_{z′}ⁱ. Similarly for \vec{w}^{j}. For zᵢ=1 and zⱼ=1 (the first elements of \vec{w}ⁱ and \vec{w}^{j}), and for design 1 in FIG. 3 (i.e. design m where m=1), partition the patients according to the corresponding trial cut-off values and the scheme of FIG. 3, to derive x_{i,j,k}ⁿ as a dichotomous variable. The algorithm then evaluates the prognostic significance of pair i, j for the cutoffs (\vec{w}_{z}ⁱ, \vec{w}_{z}^{j}) by model (1) by fitting the survival values to

log h^{i,j}ₖ(tₖ|x_{i,j,k}^{m},β_{i,j}^{m}=α_{i,j}(tₖ)+β_{i,j}^{m}·x_{i,j,k}^{m} (4)

which is the same as Eqn. (3) above. This is iterated for each of the other six designs of FIG. 3 (i.e. m=2, . . . , 7).

3. Iterate for all combinations of \vec{w}ⁱ and \vec{w}^{j} cutoffs, to find the design and the cut-off values giving the highest statistical significance value (i.e. lowest p-value).

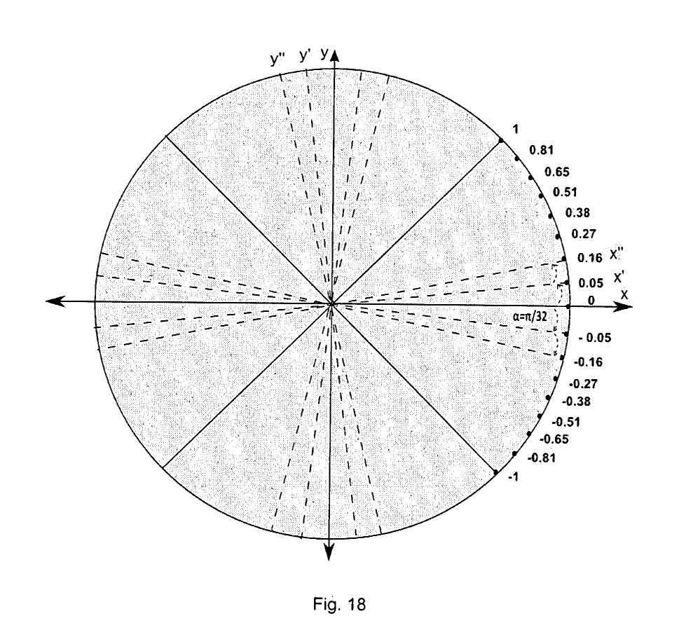

4. For each of a number of values s=1, . . . , S, define a corresponding angle αₛ. These angles are spaced apart by a regular amount such as π/32. For each value of s, rotate each of the X, Y axes by angle αₛ. This is illustrated in FIG. 18, with the angles αₛ spaced apart by π/32. The rotation works as follows:
(i) Denote the tan transformation value of an angle α in the range 0 to π as tan(α). Note that in the experiments we approximated tan(π/2)=1.63E+16.
(ii) The original axes correspond to a pair of trial cut-offs cⁱ and c^{j}. For each αₛ (s=1, . . . , S), calculate a value b₀=c^{j}+tan(αₛ)×c^{j} and use it to calculate a new X axis X′=b₀−tan(αₛ)×Y, and calculate a value b₁=c^{j}−tan(αₛ)×cⁱ giving new Y axis Y′=b₁−tan(αₛ)×X.
(iii) Using these revised axes, run 2-D DDg for all combinations of \vec{w}ⁱ and \vec{w}ⁱ cutoff pairs. Provided that the assumptions of model (1) are satisfied, the best cutoff pair and grouping scheme is the one with the smallest βᵢⱼ p-value.

5. Iterate the above steps for all i and j combinations of the N genes (i=N−1, j=i+1, . . . , N). Optionally, this may be performed only for sense-antisense gene pairs. Pairs of genes for which the result of step 4 is most significant are identified.

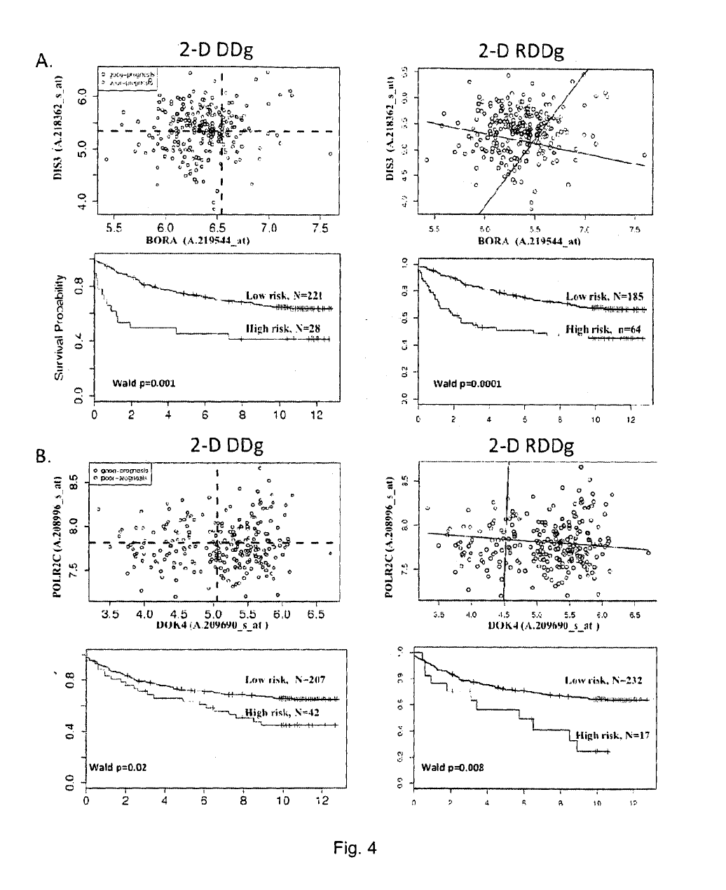

This 2-D RDDg method has a higher accuracy in grouping of patients using ccSAGPs than the 2-D DDg method because it considers the effect of significant positive correlations typical for genes-members of BCR SAGPs. Also, it makes it possible to select more optimal partitions of breast cancer patients into low-risk and high-risk subgroups. This is illustrated by FIG. 4 for patients from the Uppsala cohort where the upper parts of FIG. 4A and FIG. 4B are graphs having horizontal and vertical axes representing respectively the expression levels of two respective genes. The upper left part of FIG. 4A and FIG. 4B shows a partitioning by 2-D DDg (the optimized cut-off values are shown by dashed horizontal and vertical lines), producing a significance level of p=0.001 (FIG. 4A) and p=0.02 (FIG. 4B). The upper right part of FIG. 4A and FIG. 4B shows a partitioning by 2-D RDDg. In this case, the optimized axes are rotated relative to the axes of 2-D DDg, and the significance values are improved to 0.0001 and 0.008 respectively. The lower parts of FIGS. 4A and 4B show, respectively, the survival probability curves obtained.

Step 3 is performed for multiple cohorts of subjects (in our experiment—for two cohorts: the Uppsala and the Stockholm cohorts), to obtain respective sets of pairs of genes which are robustly survival significant using 2-D RDDg method. Step 3 is composed of step 3.1 and 3.2. In the step 3.1 the designs, rotation angles and cut-offs are chosen (to have the lowest Wald p-values for each pair) which are most optimal for all cohorts analysed and, therefore, can be more robust. We name this step also the training step.

Step 3.2 includes application of 1 D-DDg algorithm for each of the gene-members of BCR-SAGPs within total groups of breast cancer patients in order to estimate Wald p-value for each of all of the individual genes composing the ccSAGPs. Finally, those gene pairs are chosen which show lower synergistic 2-D RDDg Wald p-value as compared with 1-D DDg p-values for individual genes in all analysed cohorts (in our experiment-two cohorts). Therefore, typically, the number of survival significant ccSAGPs is expected to be less after step 3.2, than the total number of survival significant pairs extracted by applying 2-D RDDg at step 3.1.

Step 4 included application of Statistically Weighted Voting Grouping (WVG) procedure for integration of survival information for individual gene pairs into a dramatically improved patients partition. Due to the fact that the finally selected set of 3S-SAGPs showed highly significant integrated patients partition at the step 4, we named this gene pairs set as the putative sense-antisense gene classifier (SAGC). The gene pairs composing it are shown in Table 1B. Table 2 shows the p-values for the individual genes and gene pairs listed in Table 1B, to demonstrate that the test of step 3.2 was passed (refer to the first three columns under each of the headings “Stockholm cohort” and “Uppsala cohort”). Much lower integrated WVG Wald p-values (Table 2) than any of the 2-D RDDg p-values indicated that step 4 was passed as well.

Table 1B gives the host genes, Affymetrix probe sets and representative RNA transcripts for the SAGC. The best RNA ID corresponding to the Affymetrix probeset have been chosen. Priority for selection was as follows: a) best ID by chromosome coordinates; b) for the type of IDs: first, well characterized RefSeq NM IDs, then-RefSeq mRNA IDs and, finally, —EST IDs have been chosen. 1-paired transcript located on the same strand as NPC1 gene but within the territory of C18orf8 gene; 2-putative 14kD protein containing SHMT homology, clone pUS1215 from breast cancer cell line ZR-75-1; 3-fetal brain EST from cDNA clone FCBBF3000065. These three genes are indexed by superscripts in Table 1B.

Importantly, to our knowledge, none of the gene pairs composing SAGC have been suggested to be involved in breast cancer, though as individual genes, twelve out of twenty four genes composing SAGC have been reported as associated with various cancers (Table 8). That fact highlights the novelty of our approach.

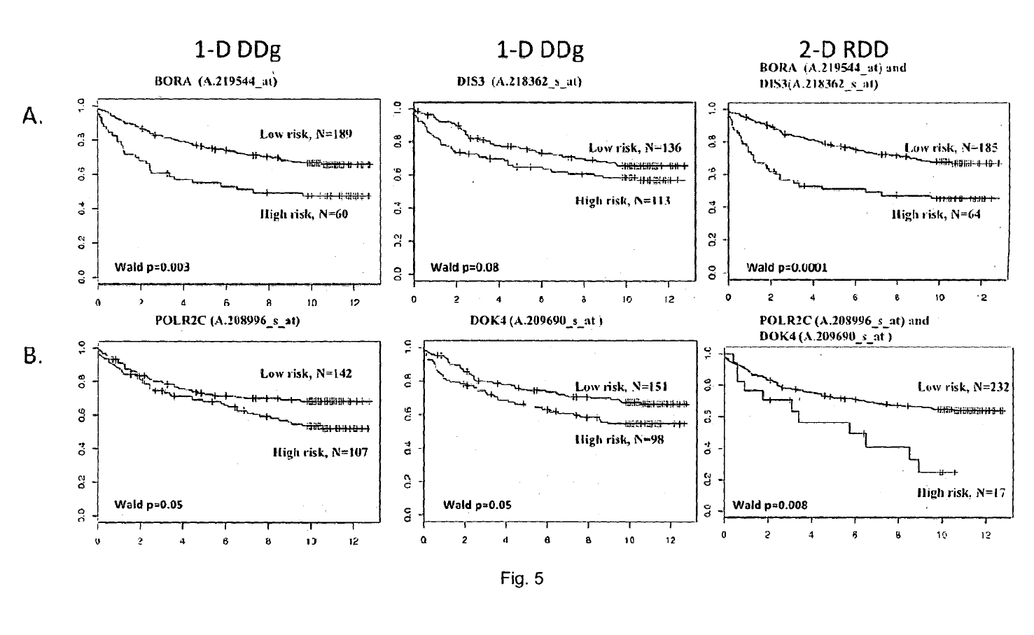

Selection of synergistic SAGPs assumes that classification of breast tumors using such gene pairs is more efficient than classification using individual genes composing ccSAGP, therefore, such gene pairs can be considered as distinct classification modules in further analyses. Thus, referring to FIG. 5, FIG. 5A gives the survival curves for two individual genes which form a pair in Table 1B, and for the pair in combination; and FIG. 5B gives the survival curves for two other individual genes which form a pair in Table 1B, and the pair in combination.

Steps 4 and 6 of FIG. 1 refer to a Weighted Voting Grouping (WVG) procedure to integrate the grouping information for 12 individual gene pairs into an integrated grouping output. The WVG is based on integrative combining of several significant or, sometimes, also non-significant features into a composite, final grouping. The algorithm of WVG is as follows:
1. Select the g significant paired features of the list sorted by the 2-D RDDg P value in ascending order. Assign to each pair g the weight to

$${w}_{g} = \frac{- {log}_{10} {p}_{g}}{{\sum }_{g = 1}^{G} - {log}_{10} {p}_{g}} ,$$

where p_{g} is the 2-D RDDg P-value of pair g, G is the total number of significant pairs (here G=12), the transformation of p_{g} into

$$\frac{1}{{p}_{g}}$$

gives more weight to the low 2-D RDDg P values (most significant pairs) and Σ_{g=1}^{G} w_{g}=1.
2. For each g calculate the group indices x₉^{(k)}×w_{g} which is a weighted grouping for each patient k. Note that x_{g}^{(k)} takes values 1 (low-risk) or 2 (high-risk).
3. For each patient k and G*=3, . . . , G estimate the summary weighted group for each patient Sₖ=Σ_{g=1}^{G*} x_{g}^{(k)}×w_{g} and run the 1-D DDg to find the cut-off that maximizes the separation of the low-risk and high risk survival curves. This cut-off determines the patient grouping of the weighted voting.
4. The best signature is the one involving G* pairs that minimize the P value of 1-D DDg (step 3 of WVG).

The WVG step allows integration of the grouping information for 12 gene pairs into a dramatically improved integrated grouping. In table 2, the numbers in the columns LR subgroup and HR subgroup are the number of individuals in these cohorts in each of the groups. The numbers were produced by RDDg, without use of the WVG step.

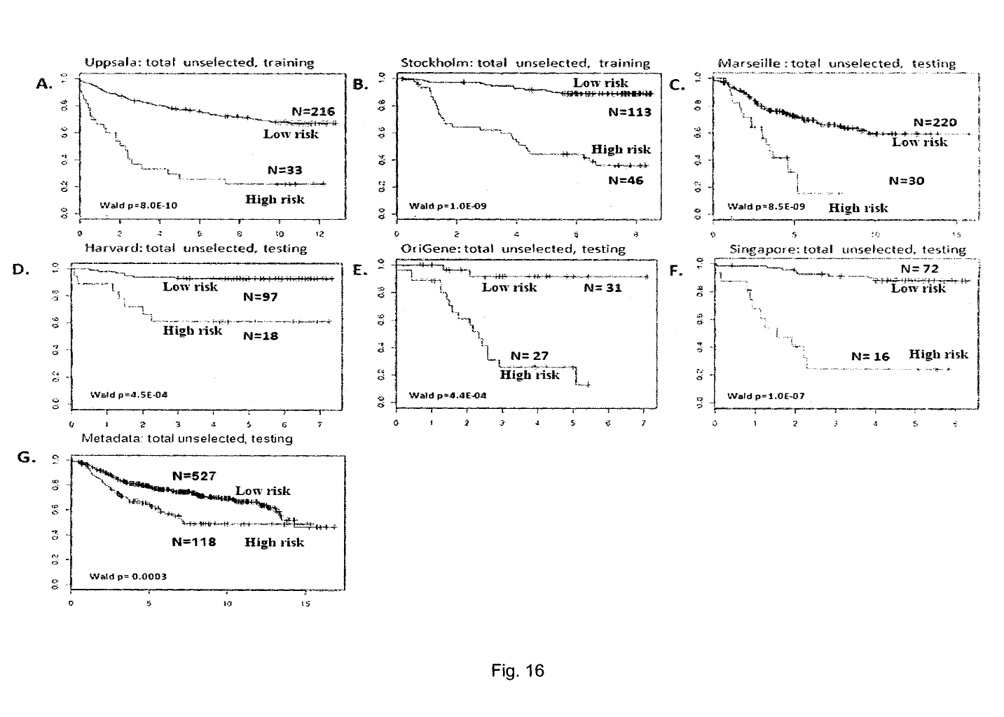

Step 5 of FIG. 1 is testing of the selected 12 SAGPs (putative SAGC classifier) in at least one independent breast cancer cohort to validate the result. Survival analysis is performed as in step 3.1, using the rotation angles and designs obtained in step 2. Grouping information on step 6 is integrated as in step 4. Because of the biological variability which is often observed between cohorts used for training and testing, strict fixation of the gene expression cutoffs in the training and the testing groups is not recommended. For the optimal partition of patients in the testing cohort, slight relaxation of the gene expression cutoff is advised. If step 6 returns such result as integrated grouping with WVG p-value less than 0.05, we conclude that the SAGC is validated for the given type of tumors. In our experiment, for total unselected breast tumors, SAGC have been validated in four independent cohorts (FIG. 16).

We now turn to FIG. 2, showing the use of the SAGC classifier obtained by the method of FIG. 1.

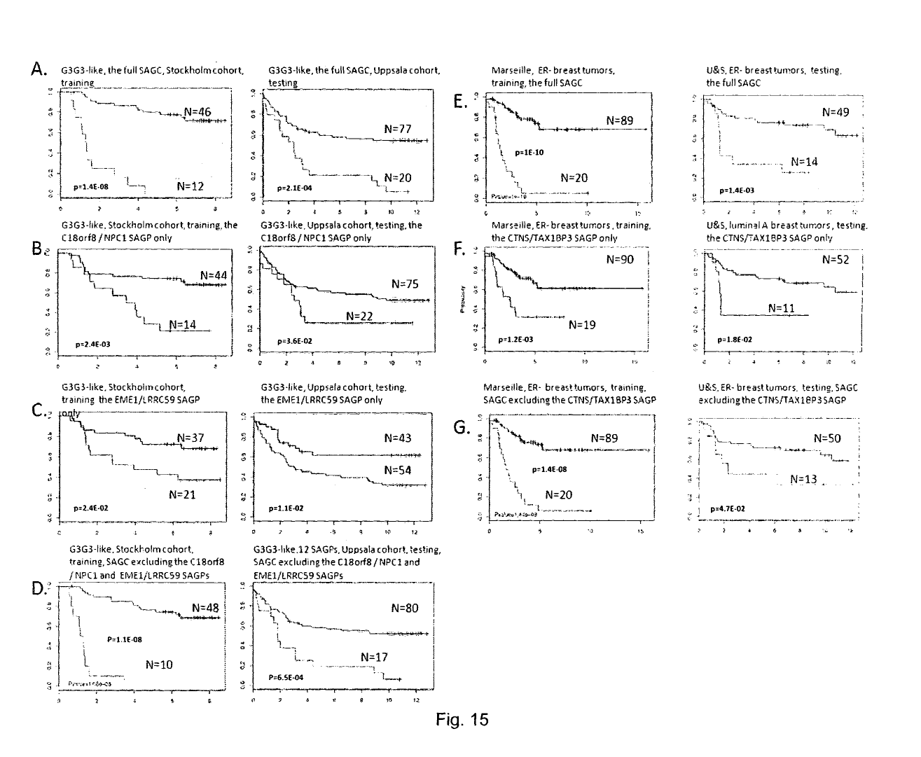

Step 7 is training and testing of the SAGC classifier for each new subpopulation or subtype of breast tumor, and comprises sub-steps 7.1 and 7.2. Sub-step 7.1 is selection of the best design, the best rotation angle and gene expression cut-offs for each of the 12 pairs of genes using the 2-D RDDg algorithm with consequent WVG procedure. The procedure is the same as in steps 3 and 4 (FIG. 1) except that no further filtering of the gene pairs is performed. Sub-step 7.2 is performed as in steps 5 and 6 (testing). Typically, the individual gene pairs which are survival significant in the training and the testing can be used as tumors classifiers; they represent the “core” SAGPs for the given tumors subpopulation. Their usage together with the rest of the signature is more efficient and robust after applying the WVG procedure (FIG. 15).

For example, within G3, G3-like breast tumors, application of the full SAGC leads to a substantially better patients partition into high-risk and low-risk subgroups (FIG. 15, A as compared with the only one SAGP(C18orf18/NPC1)(FIG. 15, B) or with only one SAGP(EME1/LRRC59) (FIG. 15, C) applied to the same tumors sample. Alternatively, excluding those SAGPs from classifier returns slightly worse but still significant patients partition in the testing experiment (FIG. 15, D, right panel). Similarly, in the ER “−” breast tumors sample, patients partition using the only one SAGP(CTNS/TAX1BP3) returns worse results (FIG. 15, F) than the full SAGC (FIG. 15, F).

The rest of FIG. 2 shows sixteen example methods in which the SAGC classifier can be used. The SAGC classifier may be used in any one of the examples shown, or in more than one.

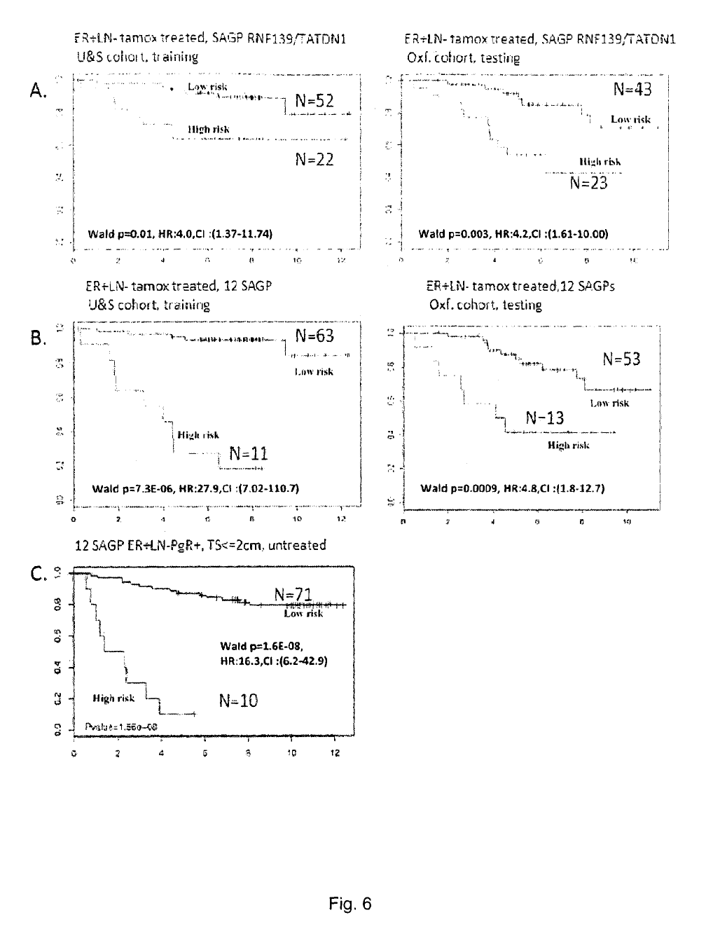

Step 8. A method for stratification and prediction of clinical outcome of ER“+”, LN“−” breast cancer patients who received adjuvant systemic tamoxifen treatment after curative surgery using the two-gene (SAGP) classifier RNF139/TATDN1. The results are shown in FIG. 6A and in Table 5. Though they represent the core SAGPs for the given tumors subpopulation, their usage together with the rest of the signature is more efficient and robust. The method includes estimation of the optimal cut-offs for expression values for each of the two genes, the optimal design and rotation angle using 2-D RDDg algorithm in one training cohort composed of at least 50 breast cancer patients with consequent testing in at least one cohort composed of at least 50 patients. Reference [38] addressed a similar problem with the two-gene expression ratio (HOX13:IL17BR).

Step 9. A method for stratification and prediction of clinical outcome of ER“+”, LN“−” breast cancer patients received adjuvant systemic tamoxifen treatment after curative surgery using SAGC classifier (12 gene pairs, 24 genes). The results are shown in FIGS. 6B and 6C. The method includes estimation of the optimal cut-offs for expression values for each of the twenty four genes, the optimal designs and rotation angles using 2-D RDDg algorithm in all 12 SAGPs in one training cohort composed of at least 50 breast cancer patients with consequent testing in at least one cohort composed of at least 50 patients. The optimal classification parameters for all 12 ccSAGPs are presented in Table 7, A. Reference [39] addressed the same problem with the Oncotype DX Assay (21 genes).

Step 10. A method for stratification and prognosis of clinical outcome of breast cancer patients with grade 3 tumors using VPRBP/RBM15B SAGP (Table 5) as well as the full SAGC classifier (12 gene pairs, 24 genes). The results are shown in FIG. 7. It includes estimation of the optimal cut-offs for expression values for each of the genes, the optimal design and rotation angle using the 2-D RDDg algorithm in one training cohort composed of at least 50 patients with consequent testing in at least one cohort composed of at least 50 patients. The optimal classification parameters for all 12 ccSAGPs are presented in Table 7, B. Reference [40] addressed the same problem with a molecular cytogenetic classifier.

Step 11. A method for stratification and prognosis of clinical outcome of breast cancer patients with grade 3 and grade 3-like tumors using SAGPs C18orf8/NPC1 and EME1/LRRC59 (Table 5) as well as the full SAGC classifier (12 gene pairs, 24 genes). The results are shown in FIG. 8. It includes estimation of the optimal cut-offs for expression values for each of the genes, the optimal design and rotation angle using the 2-D RDDg algorithm in one training cohort composed of at least 50 patients with consequent testing in at least one cohort composed of at least 50 patients. The optimal classification parameters for all 12 ccSAGPs are presented in Table 7, C. We are not aware of a similar method.

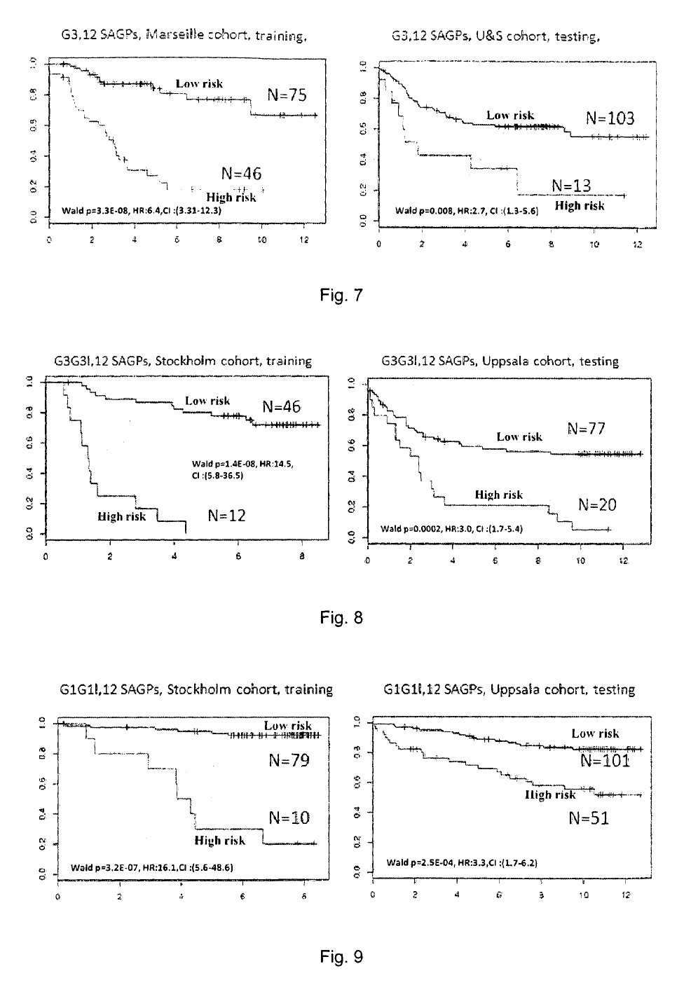

Step 12. A method for stratification and prognosis of clinical outcome of breast cancer patients with grade 1 and grade 1-like tumors using SHMT1/SMCR8 SAGP (Table 5) as well as the full SAGC classifier (12 gene pairs, 24 genes. The results are shown in FIG. 9. It includes estimation of the optimal cut-offs for expression values for each of the genes, the optimal design and rotation angle using 2-D RDDg algorithm in one training cohort composed of at least 50 patients with consequent testing in at least one cohort composed of at least 50 patients. The optimal classification parameters for all 12 ccSAGPs are presented in Table 7, D. We are not aware of a similar method.

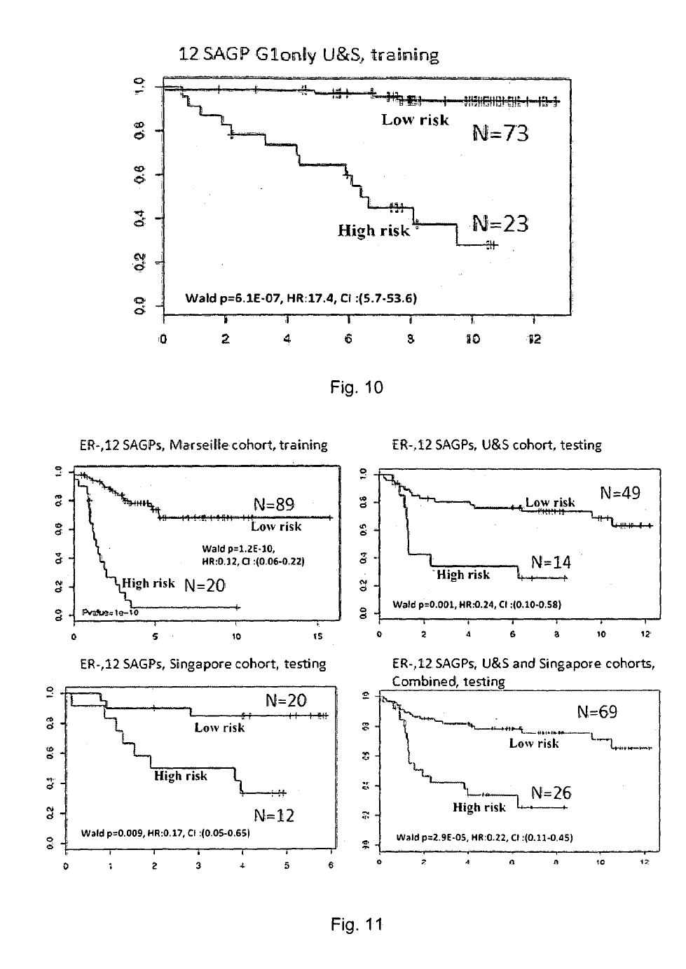

Step 13. A method for stratification and prognosis of clinical outcome of breast cancer patients with grade 1 breast tumors using the full SAGC classifier (12 gene pairs, 24 genes). The results are shown in FIG. 10. It includes estimation of the optimal cut-offs for expression values for each of the genes, the optimal design and rotation angle using the 2-D RDDg algorithm in one training cohort composed of at least 50 patients with consequent testing in at least one cohort composed of at least 50 patients. The optimal classification parameters for all 12 ccSAGPs are presented in Table 7, E. We are not aware of a similar method.

Step 14. A method for stratification and prognosis of clinical outcome of ER“−”, breast cancer patients from total unselected groups using the CTNS/TAX1BP3 SAGP (Table 5) as well as the full SAGC classifier (12 gene pairs, 24 genes). The results are shown in FIG. 11. It includes estimation of the optimal cut-offs for expression values for each of the twenty four genes, the optimal designs and rotation angles using the 2-D RDDg algorithm for each of the genes in one training cohort composed of at least 50 breast cancer patients with consequent testing in at least one cohort composed of at least 50 patients. The optimal classification parameters for all 12 ccSAGPs are presented in Table 7, F. Reference [41] addressed a similar problem using a seven-gene immune response module.

Step 15. A method for stratification and prognosis of clinical outcome of breast cancer patients with basal-like grade 3 (G3) breast tumors using the SAGPs CTNS/TAX1 BP3 and RNF139/TATDN1 (Table 5) as well as the full SAGC classifier (12 gene pairs, 24 genes). It includes estimation of the optimal cut-offs for expression values for each of the twenty four genes, the optimal designs and rotation angles using the 2-D RDDg algorithm for all the genes in one training cohort composed of at least 50 breast cancer patients with consequent testing in at least one cohort composed of at least 50 patients. The optimal classification parameters for all 12 ccSAGPs are presented in Table 7, G. Reference [42] addressed the same problem using a 14-gene signature (14 genes), and Reference [15] addressed it using a 28-kinase metagene classifier (28 genes).

Step 16. A method for stratification and prognosis of clinical outcome of breast cancer patients with Luminal A breast tumors using the BIVM/KDELC1 SAGPs (Table 5) as well as the full SAGC classifier (12 gene pairs, 24 genes). It includes estimation of the optimal cut-offs for expression values for each of the twenty eight genes, the optimal designs and rotation angles using the 2-D RDDg algorithm in all 12 SAGPs in one training cohort composed of at least 50 breast cancer patients with consequent testing in at least one cohort composed of at least 50 patients. The optimal classification parameters for all 12 ccSAGPs are presented in Table 7, H. Reference [14] addressed the same problem using a sixteen kinase gene expression classifier.

Step 17. A method for stratification and prognosis of clinical outcome of ER“+”, LN“−”, PgR“+” breast cancer patients with breast tumors <=2 cm at the time of curative surgery who usually do not receive any systemic treatment, using the SAGC classifier (12 gene pairs, 24 genes). It includes estimation of the optimal cut-offs for expression values for each of the twenty four genes, the optimal designs and rotation angles using the 2-D RDDg algorithm in all 12 SAGPs in one training cohort composed of at least 50 breast cancer patients. The optimal classification parameters for all 12 ccSAGPs are presented in Table 7, I. We are not aware of a similar method.

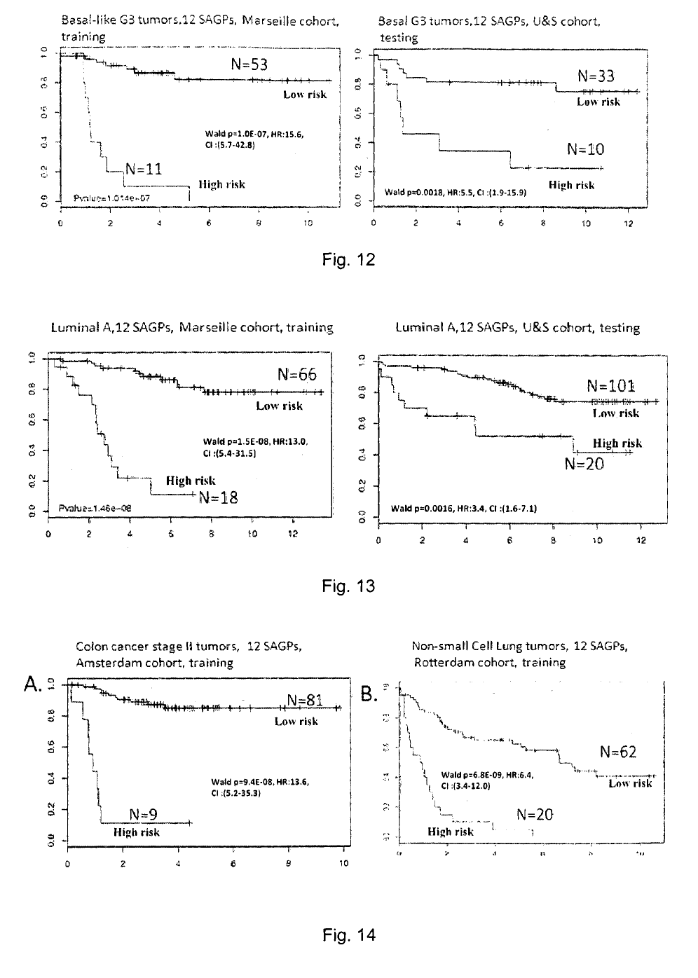

Step 18. A method for stratification and prognosis of clinical outcome of colon cancer patients with stage II tumors using the SAGC classifier (12 gene pairs, 24 genes). Results are shown in FIG. 14A. It includes estimation of the optimal cut-offs for expression values for each of the twenty four genes, the optimal designs and rotation angles using the 2-D RDDg algorithm in all 12 SAGPs in one training cohort composed of at least 50 colon cancer patients. The optimal classification parameters for all 12 ccSAGPs are presented in Table 7, J. Reference [43] addressed the same problem using a colon cancer stem cell gene signature.

Step 19. A method for stratification and prognosis of clinical outcome of non-small lung cancer patients from total unselected group using the SAGC classifier (12 gene pairs, 24 genes). It includes estimation of the optimal cut-offs for expression values for each of the twenty four genes, the optimal designs and rotation angles using the 2-D RDDg algorithm in all 12 SAGPs in one training cohort composed of at least 50 non-small lung cancer patients. The optimal classification parameters for all 12 ccSAGPs are presented in Table 7, K. Reference [44] addressed the same problem with a non-small lung cancer 17-gene signature.

Step 20. A method for stratification and prognosis of clinical outcome of breast cancer patients from original/total unselected group using the SAGC classifier (12 gene pairs, 24 genes). It includes estimation of the optimal cut-offs for expression values for each of the twenty four genes, the optimal designs and rotation angles using the 2-D RDDg algorithm in all 12 SAGPs in one training cohort composed of at least 50 breast cancer patients. The optimal classification parameters for all 12 ccSAGPs are presented in Table 7, L.

Step 21. A method for identification of SAGC classification-associated biomarkers of breast tumor heterogeneity which are specific and reliable in a context of patient survival, as well as mechanistically related biomarkers mostly appropriate for therapeutic targeting. The method includes the following steps:
i) obtain gene expression data for at least two independent groups of cancer patients with a given cancer and retrospective post-operation survival data (e.g., total unselected cohort);
ii) in each cohort, classify breast cancer patients into low-risk and high-risk subgroups using the workflow described in steps 3-6 of FIG. 1 and in step 7 of FIG. 2;
iii) stratify patients into the disease risk subgroups in each unrelated cohort using the prognostic model and our algorithm
iv) identify the robust differentially expressed genes (DEG) defined as a common sub-set of DEGs derived with the same disease prognosis model of patient's stratification and found in all studied unrelated cohorts;
v) identify high-confidence overrepresented gene ontology categories within the list of the robust DEGs using Functional Gene Annotation/Gene Ontology enrichment analysis (e.g., Database for Annotation, Visulization and Integrated Discovery (DAVID) Bioinformatics tools, http://david.abcc.ncifcrf.gov/) and/or network analysis (e.g. MetaCore; GeneGo of Thomson Reuters, http://portal.genego.com) providing a set of mechanistically-driven gene subsets and gene networks, allowing finally to select one or more prognostic signatures with mechanistic interpretation of patho-biological changes in the cancer-related and robust differentially expressed genes, collectively associated with the identified gene subset(s).
vi) using manual literature curation, publicly and commercially available drug target databases, identifying novel/prospective and known biomarkers within the identified mechanistic-driven gene signature, containing the most appropriate molecular targets for optimal therapeutic intervention.

The method has been successfully used to identify breast cancer patients with distinct prognosis of breast cancer recurrence (as shown below). We apply our method to two original total (unselected) breast cancer patient cohorts (Uppsala and Stockholm cohorts (training) as well as to Marseille, Harvard 2, Singapore and OriGene cohorts (testing)). The optimal parameters of SAGC for original cohorts are presented in Table 7L.

The method can be also applied to a patient subpopulation with a given tumor subtype shown to be heterogeneous upon application of SAGC and described in the steps 9-19 above. Because the tumors in subpopulations/subtypes are biologically more homogeneous than the tumors in original unselected cohorts, for the identification of robust DEGs and associated mechanistically-related and therapeutic biomarkers, at least three independent patient groups with size at least 100 patients in each is recommended. We are not aware of a similar method.

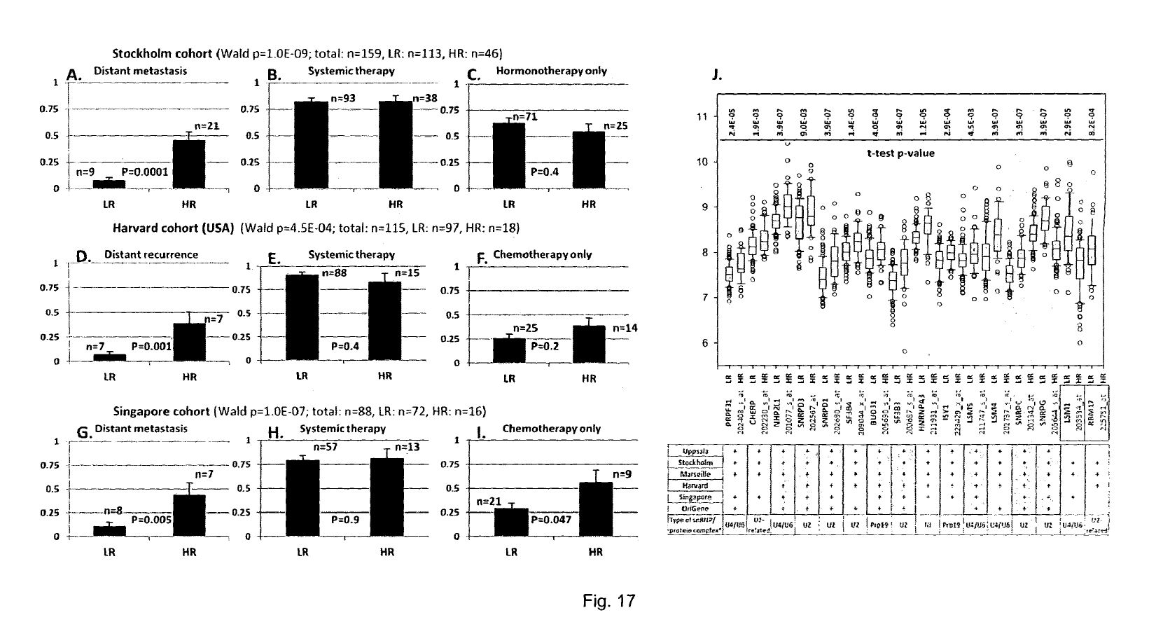

Step 22. A method for identification of specific HR subgroups (with a relative upregulation of “proteasome- and spliceosome-enriched” genes associated with poor prognosis of breast tumors) of breast cancer patients from original/total unselected groups using SAGC and method described on Step 20. Results of application of this method are shown in Table 10 and FIG. 17. FIG. 17A-I show the effect of different treatment modalities (chemo- and hormonotherapy) on HR subgroup separated by SAGC in three independent cohorts; FIG. 17.J shows an example of 14 genes involved in precatalytic spliceosome complex B robustly overexpressed in HR subgroups in six studied cohorts (919 patients). The upper panel shows overexpression of the genes in HR vs. LR subgroups in the Stockholm cohort. The genes in boxes are LSM1 (oncogene and potential drug target) and RBM17 (confers multidrug resistance upon overexpression) shown for comparison. The lower panel summarizes overexpression data for six independent cohorts. “+”—indicates that the given gene is significantly overexpressed in HR subgroup of the given cohort with t-test p-value <0.05. The seven most robust genes are in grey. Reference for the type of snRNP confirms that all 16 genes shown belong to the same specific stage: precatalytic spliceosome complex B.

That specific subgroup is characterized by: i) significantly higher rate of distant metastases/distant recurrence; ii) resistance to chemotherapy and hormonotherapy (FIGS. 17C, F and I); iii) GO term(s) enrichment of deregulated (overexpressed) genes belonging to the specific stage of splicing cycle-precatalytic stage of spliceosome assembly or complex B (see below with reference to FIG. 17J and to Table 10).

Step 23. A method for identification of specific HR subgroups (with “proteasome-” and “spliceosome-enriched” breast tumors) of breast cancer patients from original/total unselected groups of breast tumors using genes of proteasome and/or spliceosome complex B in breast tumors. The method includes computational procedures on steps 3-6 in FIG. 1 of the current invention to any gene pairs (not necessarily, sense-antisense gene pairs) composed of the proteasome or spliceosome genes from Tables 10. This method is a generalization to the method reported on Step 21. Identification of patients with “proteasome-” and “spliceosome-enriched” tumors could be beneficial for the development of the mechanistic-driven prognostic and prediction methods which consequently lead to the tailoring of adjuvant treatment plans based on anti-tumor drugs targeting proteasome and spliceosome (and, specifically, precatalytic stage of spliceosome). This mechanistically-driven patient survival prognosis model could be potentially effective while it uses the same combined biomarker for the disease prognosis and treatment prediction of the tumors having overrepresented and overexpressed genes of spliceosome machinery. Such schemes would use the combination of conventional drugs targeting cell cycle and DNA replication, hormone therapy as well as agents targeting specific components of spliceosome machinery. For example, transient, short-term treatments after surgery with drugs specifically targeting the spliceosome, the fidelity of the splicing process [45] and, more specifically, precatalytic stage of spliceosome assembly, might not lead to dramatic drug side effects due to their selective tumor cytotoxicity [46, 47]. Although it could definitely increase the tumor's sensitivity for the consequent standard chemotherapy treatment [47]. Andre et al [4] have addressed the same problem using a high-dimensional (1228-probe set) molecular classifier.

Step 24. A method for identification of novel drug targets using SAGC and their implication. In the current proposal, we identified the certain genes of proteasome and spliceosome as novel prospective therapeutic target(s) in primary breast tumors which were classified as “proteasome-” and “spliceosome-enriched” HR subtype and were revealed using SAGC. We propose that existing or novel drugs which could be used for the treatment breast cancer patients belonging to the “proteasome-” and “spliceosome-enriched” subgroup can be identified based our prognostic method and our SAGC. The “proteasome-” and “spliceosome-enriched” subtype of breast tumors could be sensitive to: i) anti-spliceosome drugs belonging to the GEX1 group [48]; ii) synthetic compounds spliceostatin A, meayamycin, meayamycin B and their derivatives which target U2 snRNP and block spliceosome complex A formation [49]; iii) groups of compounds called sudemycins and their derivatives; iv) groups of compounds called pladienolides and their derivatives, such as E7107; v) compound isoginkgetin and its analogs targeting precatalytic stage of spliceosome assembly and inhibiting the A to B spliceosome complex transition [50]; vi) anti-proteasome drugs targeting i) the 20S proteolytic proteasome subunit (such as Bortezomib); ii) the 19S proteolytic proteasome subunit (such as b-AP15).

We are aware of two similar developments. Firstly, a study in which it has been shown that anti-LSM1(anti-oncogene) antisense gene therapy can be effective in vitro (pancreatic cell line) and in vivo (SCID-Bg mice) for pancreatic cancer treatment [51, 52]. Specifically, a single intramural injection of an adenoviral vector expressing a 900-base pair antisense RNA to CaSm (LSM1) directly to subcutaneous AsPC-1 tumors reduced in vivo tumor growth by 40% and extended median survival time from 35 to 60 days [51]. Secondly, a study in which treatment of human breast cancer MCF-7 cells by synthetic compounds FR901464 and meayamycin specifically targeting spliceosome (and, namely SF3b complex) inhibited their proliferation [53]. These results provide independent support of our spliceosome signature, deduced via prognostic method presented in this specification (see Steps 20-24).

Step 25. A method for detecting multidrug-resistant tumors (i.e., resistant to chemo- and hormonotherapy) in primary breast tumors using the genes of precatalytic stage of spliceosome assembly (complex B). Increased level of gene expression for those 14 genes in breast cancer patients indicates the phenotype of resistance to standard chemo- or hormonotherapy. In Reference [54] the authors have addressed the same problem, and showed that the over-expression of the U2-related splicing component RBM17 (SPF45) could be the causative factor and indicator of multidrug-resistant phenotype in HeLa cells. These results support our identification of the 14-gene spliceosome signature and its importance as a mechanistically-driven complex prognostic biomarker.

### Advantages of the Embodiments Over Existing Technologies

### Practical Advantages

1) The proposed two-gene classifier RNF139/TATDN1 achieved similar or higher accuracy in prediction of clinical outcome and stratification of ER“+”, LN“−” breast cancer patients who received systemic tamoxifen treatment—to the two-gene expression ratio (HOX13:IL17BR) [38, 55]. The SAGC classifier outperformed the HOX13:IL17BR classifier in the testing experiment (lower log-rank p-value, larger difference for 5-year- and 10-year DFS between LR and HR subgroups). See FIG. 6A, and Tables 3A1 and 3A2, example 1.

2) The SAGC classifier (12 gene pairs, 24 genes) achieved substantially higher accuracy in prediction of clinical outcome and stratification of ER“+”, LN“−” breast cancer patients who received systemic tamoxifen treatment than the Oncotype DX Assay (21 genes) [39]. The SAGC classifier outperformed the Oncotype DX Assay: lower likelihood ratio p-values and larger differences for 5-year- and 10-year DFS between LR and HR subgroups both in the training and testing experiments. See FIG. 6B, and Tables 3A1 and 3A2, example 2.

3) The SAGC classifier (12 gene pairs, 24 genes) achieved substantially higher accuracy in prognosis of clinical outcome and stratification of breast cancer patients with grade 3 tumors. The SAGC classifier outperformed the molecular cytogenetic classifier: dramatically lower log-rank p-value and larger differences for 5-year- and 10-year DFS between LR and HR subgroups in-training experiments. See FIG. 7, and Tables 3A1 and 3A2, example 3.

4) The SAGC classifier (12 gene pairs, 24 genes) makes possible a prognosis of clinical outcome and stratification of breast cancer patients with grade 3 and grade 3-like tumors. This is shown in FIG. 8, and Tables 3B1 and 3B2, example 4. No other way of doing this is currently known.

5) The SAGC classifier (12 gene pairs, 24 genes) makes possible the accurate prognosis of clinical outcome and stratification of breast cancer patients with grade 1 and grade 1-like tumors. This is demonstrated by FIG. 9, and Tables 3B1 and 3B2, example 5. No other way of doing this is currently known.

6) The SAGC classifier (12 gene pairs, 24 genes) makes possible the accurate prognosis of clinical outcome and stratification of breast cancer patients with grade 1 tumors. This is demonstrated by FIG. 10, and Tables 3B1 and 3B2, example 6. No other way of doing this is currently known.

7) The SAGC classifier (12 gene pairs, 24 genes) makes possible prognosis of clinical outcome and stratification of ER“−” breast cancer patients with similar or higher accuracy than the prototype—the seven-gene classifier from Reference [41]. The SAGC classifier outperformed the corresponding prototype in the training and testing experiments (lower log-rank p-values, larger differences for 5-year- and 10-year RFS/DFS between LR and HR subgroups). This is demonstrated in FIG. 11, and Tables 3B1 and 3B2, example 7.

8) The SAGC classifier (24 genes) provides higher accuracy in prognosis of clinical outcome and stratification of breast cancer patients with basal-like grade 3 (G3) breast tumors as compared with 2 prototypes—the 14-gene signature (14 genes) from Reference [42] and the 28-kinase immune metagene (28 genes) from Reference [15]. The SAGC classifier outperformed the prototype 1 in the testing experiment (lower log-rank p-value). It outperformed the prototype 2 (lower log-rank p-values in the training experiment, larger differences for 5-year RFS/DFS between LR and HR subgroups). See FIG. 12 and Tables 3B1, 3B2, 3C1 and 3C2, example 8.

9) The proposed SAGC classifier (24 genes) provided substantially higher accuracy in prognosis of clinical outcome and stratification of breast cancer patients with Luminal A breast tumors as compared with the prototype-sixteen kinase gene expression classifier from Reference [14]. SAGC classifier outperformed the corresponding prototype in the training and testing experiments (lower log-rank p-values, larger differences for 5-year- and 10-year RFS/DFS between LR and HR subgroups). See FIG. 13, and Tables 3C1 and 3C2, example 9.

10) The SAGO classifier (12 gene pairs, 24 genes) made it possible to predict the clinical outcome and stratify breast cancer patients with generally favorable prognosis: ER“+”, LN“−”, PgR“+” patients with tumors <=2 cm who usually do not receive systemic chemo- or tamoxifen therapy. See FIG. 6C, and Tables 3C1 and 3C2, example 10.

11) The proposed SAGO classifier (24 genes) permitted substantially higher accuracy in prognosis of the clinical outcome and stratification of colon cancer patients with stage tumors as compared with the prototype-colon cancer stem cell gene signature from Reference [43]. The SAGC classifier outperformed the corresponding prototype in the training experiment (lower log-rank p-values, larger differences for 5-year RFS between LR and HR subgroups). See FIG. 14A, Tables 3C1 and 3C2, example 11.

12) The proposed SAGC classifier (24 genes) provided substantially higher accuracy in prognosis of clinical outcome of non-small lung cancer patients from total unselected group as compared with the prototype-non-small lung cancer 17-gene signature from Reference [44]. The SAGC classifier outperformed the corresponding prototype in the training experiment (lower log-rank p-values, larger differences for 5-year and 10-year OS between LR and HR subgroups). See FIG. 14B, and Tables 3C1 and 3C2, example 12.

13) The SAGC classifier (12 gene pairs, 24 genes) made possible identification of novel biomarkers of breast tumors heterogeneity as well as novel drug targets using SAGC.

14) The SAGO classifier (12 gene pairs, 24 genes) made possible identification of breast tumors (breast cancer patients) with “proteasome-” and “spliceosome-enriched” BC subtype characterized by: i) high rate of distant recurrence/distant metastases; ii) resistance to chemo- and hormonotherapy; iii) overrepresented deregulated (overexpressed) genes of proteasome and spliceosome (see FIG. 17J and Table 10). Consider a currently known prototype—a 1228-probeset molecular classifier from Reference [4]. Similarly to the SAGC classifier, the 1228-probeset classifier is able to identify breast cancer samples with differential expression of spliceosome genes. However, the SAGC has the following advantages: i) 1228-probeset classifier have been specifically designed to improve the diagnosis of breast tumors, i.e. by distinguishing between benign lesions (normal breast tissue) and malignant breast tumors and it may not be suitable (if otherwise, special study must be provided) for prognostic identification within malignant breast tumors, i.e. by distinguishing between high-metastatic and low-metastatic malignant tumors; ii) prototype uses 1228 discriminative features for classification while SAGC-only 24; therefore, the SAGC is much easier to implement as a routine laboratory assay; iii) the prototype classifier is based on supervised approach and is only useful for identification of predetermined and already known (e.g., benign vs. malignant) breast tissue subpopulations, while the SAGC is based on an unsupervised approach and, hence, can be used to identify previously unknown genetically and clinically distinct breast tumors subtypes; iv) The SAGC classifier identifies tumors with overexpression of specific genes of proteasome and spliceosome, and that fact can be crucial for development and/or implication of novel and already existing drugs, specifically targeting the proteasome or spliceosome.

15) The experimental results obtained from the embodiment suggested the possibility of using the genes of proteasome and spliceosome for identification of tumors of “proteasome-” and “spliceosome-enriched” BC subtype by application gene pairs composed of any of those genes in Table 10 to procedures in steps 3-6 in FIG. 1 as an alternative to usage of the SAGC.

16) The experimental results obtained from the embodiment suggested the possibility of using the genes spliceosome as robust biomarkers for detecting breast tumors with multidrug resistance (i.e., chemo- and hormonotherapy) corresponding to HR subgroups selected by SAGC in primary breast tumors. As shown in FIG. 17J, the proposed prototype—U2 snRNP-related splicing component RBM17 (SPF45) is less robust in primary breast tumors (significantly overexpressed in HR subgroups in at least 3 cohorts out of 7 tested) than 14 others identified in the current invention. The extensive application of the SAGC in 6 independent cohorts with diverse ethnic composition (totally, 919 patients, FIG. 17, J) reveals at least 8 other genes (highlighted in grey) of spliceosome which show more reliable overexpression in HR subgroups as compared with LR subgroups (significantly overexpressed in HR subgroups in 6 cohorts out of 6 tested).

17) The experimental results obtained using the embodiment suggested the possibility of using the genes of proteasome and spliceosome as potential drug targets for treatment of breast cancer patients with “proteasome-” and “spliceosome-enriched” subtype of breast tumors (see method 20 above). In the similar development 1 (see method 20 above) another gene of U4/U6 snRNP(LSM1) was proposed as antisense RNA therapy target for treatment of pancreatic but not breast cancer. At least eight genes of precatalytic stage of spliceosome showed more robust overexpression than LSM1 in “spliceosome enriched” breast tumors. In the similar development 2 (see method 33 above) the study was performed using MCF-7 breast cancer cell lines; in the current proposal the primary breast tumors have been studied. Our focus was the breast tumors belonging specifically to “proteasome-” and “spliceosome-enriched” subtype. Similar development 2 focused targeting SF3B complex using drugs FR901464 and meayamycin targeting spliceosome complex A; in our proposal we also suggest targeting precatalytic stage of spliceosome (complex B) by drug isoginkgetin or its analogs.

### Microarray Analysis

Total RNA was obtained for 58 breast cancer patients from OriGene Technology (Rockville, Md.). Agilent 2100 bio analyzer was used to check the quality of selected total RNA. All the RNA samples used for microarray studies had a RIN value above 8 indicating good quality of RNA. The GeneChip 3′ In vitro transcription (IVT) protocol that includes Reverse transcription to synthesize First strand cDNA, Second-strand cDNA, Biotin-modified mRNA labeling, mRNA purification and fragmentation were carried out using Affymetrix manufacturer's protocol. A total of 500 ng of RNA was used for the above procedures. Positive control RNA provided by the manufacturer was included for quality control check. Hybridization, subsequent washing, and staining of the arrays were carried out as outlined in the GeneChip® Expression Technical Manual. 62 Affymetrix GeneChip® Human Genome U133 Plus 2.0 oligonucleotide chips were used for gene expression analysis. Hybridization was carried out for 16 h; washing and staining were undertaken in Affymetrix Fluidics Station 450 workshop. Probe arrays were scanned using Affymetrix GeneChip Scanner 3000, covering 47,000 transcript variants, containing over 38,500 function-known genes, based on databases (GenBank, dbEST, RefSeq, UniGene database (Build 159 Jan. 25, 2003), Washington University EST trace repository, NCBI human genome assembly (Build 31)).

### Validation of SAGC.

Biological validation of SAGC was performed in the total unselected groups in the testing groups (FIGS. 16, C, D, E and F) as well as in various diverse specific BC subgroups (FIGS. 6, 7, 8, 9, 11, 12 and 13). In each case the optimal parameters (design, rotation angle and two gene expression cutoffs) selected in certain BC groups/subgroups (training mode) were fixed and applied in the testing groups (testing mode) microarray datasets from independent clinical centers. Batch effect correction between training and testing BC groups/subgroups were performed using ANOVA model.

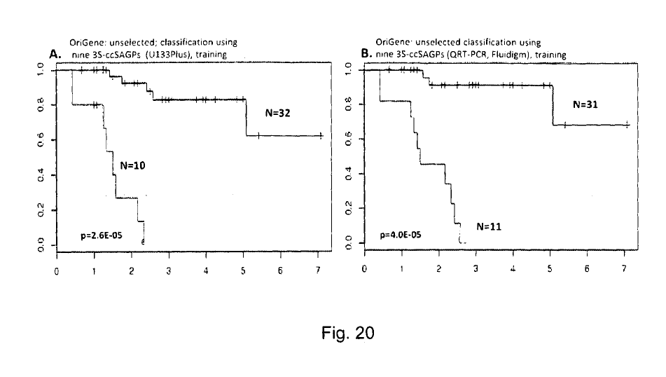

For technical validation of SAGC, the selected ccSAGPs identified using microarray data were validated using strand-specific QRT-PCR. We designed a protocol for strand-specific QRT-PCR for nine out of twelve SAGPs (eighteen genes, Table 11) in order to exclude undesirable noisy signal for gene expression from an opposite DNA strand within the regions of sense-antisense overlaps. Classification of forty two unrelated breast tumors purchased from OriGene (OriGene Technologies, Rockville, Md.) was performed in parallel using the U133Plus microarray expression data (FIG. 20, A) and the QRT-PCR expression data for the same genes and patients (FIG. 20, B). The 2-D RDDg and WVG procedures in the training mode were independently applied to both datasets. The two independent methods of gene expression detection showed strong concordance in the partitions determined for the patients (Cohen's Kappa=0.56, p=0.001). Therefore, we developed a prototype of the first QRT-PCR-based sense-antisense gene pairs assay. The advantages of our multigene assay included the use of the extreme computational procedures for efficient survival analysis (2-D RDDg and WVG) as well as use of the microfluidic high-throughput Fluidigm technology for accurate and fast expression detection for many genes at a time.

### Strand-Specific Quantitative RT-PCR

cDNA synthesis was carried out for 42 total RNAs (250 ng) of breast cancer patient samples purchased from Origene Technologies (Rockville, Md.) using a gene-specific pool of reverse primers specific for the regions of sense/anti-sense transcripts in separate reactions. Oligoprimers were selected based on being located within specific regions spanned by corresponding Affymetrix probesets. Pre-amplification step for sense/anti-sense cDNAs of 42 patient samples was conducted (LifeTechnologies, Taqman PreAmp Master Mix kit) using a gene-specific pool of sense/anti-sense of forward and reverse primers by including actin beta (ACTB) and TATA box binding protein (TBP) as endogenous controls. Taqman probes were designed for all sense and anti-sense genes and also for the endogenous controls. A 96.96 Dynamic Array IFC was prepared according to the manufacturer's instructions (Fluidigm, San Francisco, Calif.) and as described in Reference [56]. Quantitative PCR was performed using a gene assay (1st BASE, Singapore), according to the protocol for the Biomark System (Fluidigm, San Francisco, Calif.). Reaction conditions were as follows: 50° C. for 2 min, 70° C. for 30 min, 25° C. for 10 min and 50° C. for 2 min and 95° C. for 10 min, followed by 40 cycles of 95° C. for 15 sec and 60° C. for 60 sec. Data processing and Ct values extraction was done by using detector threshold settings, allowing thresholds to be individually set for each gene, and linear baseline correction was performed using Biomark Real-time PCR Analysis software (v.3.0.4) (Fluidigm, San Francisco, Calif.). Relative quantification of various genes was done using the ΔΔCt method [57]. A list of forward and reverse primers for both sense/anti-sense genes along with respective fluorescent Taqman probes labeled with FAM-TAMRA quencher is shown in Table 9.

The Applicability of the SAGC for Identification of Novel Biomarkers of Breast Tumors Heterogeneity, Biomarkers of Resistance to Standard Chemo- and Hormonotherapy as Well as for Discovery of Novel Potential Drug Targets for Specific Breast Tumor Subtypes.

In order to test if SAGC can identify candidates for novel robust biomarkers of specific breast tumors subpopulations we applied SAGC for 7 independent total unselected cohorts having 1161 breast cancer patients in total. In the first step, optimal parameters for the 2-D RDDg procedure (design, rotation angle and gene expression cutoff) were chosen and fixed in the training procedure (Uppsala and Stockholm cohorts) and applied to 5 other independent testing cohorts (Marseille, Harvard, OriGene, Singapore and Metadata cohorts, FIG. 16A-G).

The second step included identification of differentially expressed genes between low-risk and high risk subgroups using EDGE software [58] in the Uppsala, Stockholm and Metadata cohorts (training cohorts for differential expression). The robust list of 1377 genes which passed the selection criteria (FDR corrected t-test Q-value<0.01) simultaneously in three cohorts were selected for further FGA/GO enrichment analysis by DAVID software. We found among 978 genes upregulated in HR subgroups within the category KEGG_PATHWAY such FGA terms as “DNA replication” (p=2.1e-10)”, “cell cycle” (p=3.3e-14), “mismatch repair” (p=1.2e-4) (Tables 6 and 11). Similarly, within the category SP_PIR_KEYWORDS we observed strong enrichments for cell division, mitosis, DNA replication and ubiquitin conjugation pathway. Importantly, among all 978 differentially expressed upregulated genes the FGA term “Proteasome” (KEGG_PATHWAY, p=5.5e-17) had showed the strongest enrichment (p=5.5E-17). Within the same category, we also observed strong enrichment for the term “Spliceosome” (p=8.5E-05). Moreover, among upregulated genes several other categories revealed various terms associated with proteasome, splicing and spliceosome: “proteasome complex” (GOTERM_CC_FAT, 9.8E-18), “mRNA splicing” (SP_PIR_KEYWORDS, p=1.3e-07), “RNA splicing” (GOTERM_BP_FAT, p=6.8e-08) and others (Table 6).

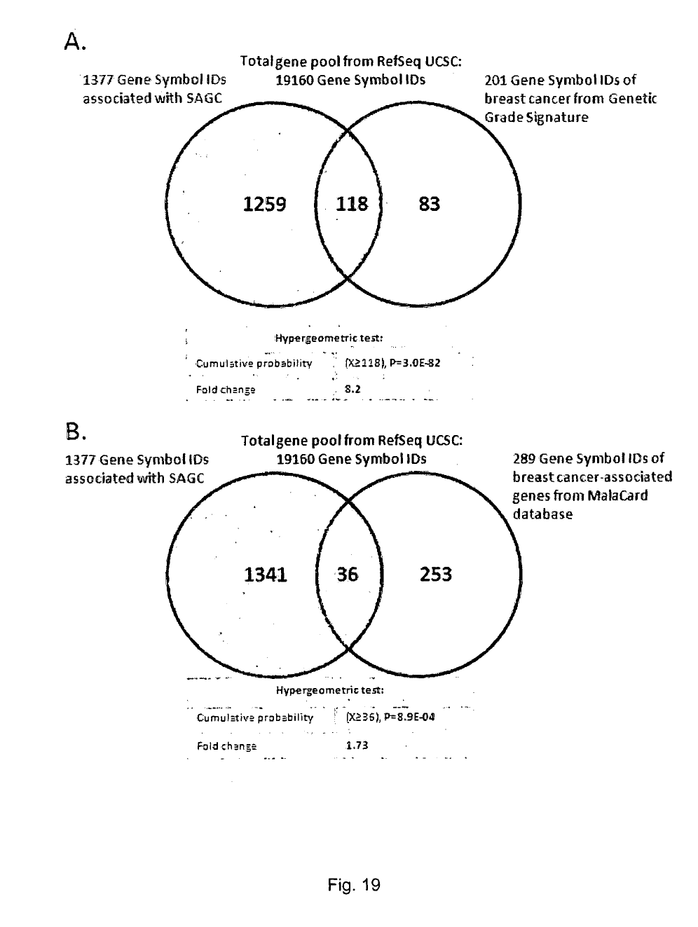

In order to get an idea how the SAGC-associated genes (i.e., differentially expressed genes between HR and LR subgroups derived by SAGC) are related to currently known breast cancer-associated genes, we compared the SAGC-associated gene set with: 1) the published gene set of Genetic Grade Signature (201 unique Gene Symbols) [22]; 2) the reliable set of 289 genes significantly associated with breast cancer from MalaCard database (http://www.malacards.org/card/breast_cancer). In the first comparison, striking enrichment (8.2 times, p=3.0E-82, FIG. 19, A) in the intersection between two sets strongly indicated that both sets must belong to the same pool of breast cancer-associated genes, though 1259 SAGC-associated genes were new. Similarly, in the second comparison, highly significant enrichment (1.73, p=8.9E-04, FIG. 19, B) in the intersection independently confirmed that SAGC-associated genes belong to the extensive pool of breast cancer-associated genes. Nevertheless, 1341 genes from SAGC-associated genes set have not been previously annotated as breast cancer-associated. We concluded that application SAGC for breast tumors classification can be efficiently used to discover a large number of potentially novel breast cancer biomarkers.

Uppsala, Stockholm and Metadata cohorts showed significant enrichment of FGA/GO terms for proteasome and spliceosome genes between HR and LR subgroups (Tables 6, 10 and 11). We suggested that HR-subgroups selected by SAGC demonstrate similar specific molecular characteristic and we proposed that they belong to the same novel subtype of breast tumors enriched by the overexpressed genes of proteasome and spliceosome. More detailed analysis revealed that the identified spliceosome genes mostly belong to the same specific stage of spliceosome cycle-precatalytic spliceosome, or complex B. Of note, this stage of splicing cycle is marked by formation of snRNP complex composed of U1-, U2-snRNPs, Prp19 complex and U4/U5/U6 tri-snRNPs and followed by the catalytic spliceosome, or active complex C, when chemical steps of splicing occur. Complex C misses the U4/U6 snRNPs [59]. The stage of complex B is also distinct from the stage of complex A where only U1- and U2-snRNPs, but not Prp19 and U4/U5/U6 tri-snRNPs are involved [59]. FIG. 17 shows 14 genes of spliceosome overexpressed in “spliceosome enriched” subtype mostly belong to the U2-, U4/U6-snRNPs or to the Prp19 protein complex.

Analysis of 27 proteasome genes (proteasome gene signature) identified under the DAVID term “hsa03050: Proteasome” revealed that they are evenly representing both the 20S core particle and the 19S regulatory particle of proteasome (Tables 6, 10 and 11). The association of the SAGC-based classification with proteasome (20S and 19S subunits) and spliceosome (precatalytic splicing) genes is interesting in context of drug targets for BC. The first anti-proteasome drug targeting the 20S proteolytic proteasome subunit, Bortezomib, was developed [60] and approved by US FDA for treatment of multiple myeloma. However, due to drug resistance, its efficiency in BC was insignificant when used as a single agent. Recently, a novel drug targeting the 19S-proteasome subunit, b-AP15, was identified and tested against several cancers [61] in mice. In contrast to Bortezomib, b-AP15 induced apoptosis regardless of mutations or deletions in TP53 or amplification of BCL2 [61]. These data suggest that the development of multigene classifiers to specifically identify and predict “proteasome-” and “spliceosome-enriched” patient subgroups could improve personalized treatment schemes in BC. In turn, these therapies could be combined with standard adjuvant therapy and known or novel anti-proteasome and anti-spliceosome drugs [60, 61, 62]

We suggested that those 25 spliceosomal and 27 proteasomal genes (Table 10) could be used for development of novel biomarker(s)/drug targets specific for the “proteasome-” and “spliceosome enriched” subtype identified by SAGC. Noteworthy, that similar scheme could be applied within other specific subpopulations of breast tumors and, correspondingly, novel biomarkers of high-risk subgroups could be identified by SAGC.

As more detailed drug treatment information has been available in the Stockholm, Harvard, OriGene and Singapore cohorts, we checked if SAGC could be useful for the assessment of drug resistance in standard treatment schemes after curative surgery. In four cohorts total percentages of patients who underwent systemic treatment (chemotherapy or hormonotherapy or both) was not different in LR and HR subgroups (FIGS. 17B, E and H, OriGene cohort not shown). Although, in HR subgroups, the percentages of patients who received only chemotherapy were significantly (Singapore and OriGene cohort) or non-significantly (Harvard cohort) higher than in LR subgroups indicating the presence of chemoresistance in HR subgroups (FIGS. 17I and F). In HR-subgroup of Stockholm cohort (FIG. 17C) resistance to hormonotherapy was observed. These findings are interesting because previously it has been shown that deregulation of certain splicing factors (such as RBM17/SPF45 or SF3B1) may confer multidrug resistance in cancers [54, 63]. Importantly, among ten genes encoding spliceosome components and robustly over-expressed in HR subgroups in 6 independent breast cancer cohorts, two-SF3B4 (SAP49) and SF3B3(SAP130)-belong to the same SF3b protein complex as an important specific sub-component of spliceosome (U2-snRNP). The SF3b complex represents specific interest because it has been actively studied as potential promising anticancer drug target [53, 64]. E.g., Spliceostatin A (FR901464) is a potent antitumor natural product that binds to the SF3b complex and inhibits pre-mRNA splicing in vitro and in vivo [65]. An analogue of FR901464, meayamycin is even more effective as an antiproliferative agent against human breast cancer MCF-7 cells [64]. As a consequence, specific splicing changes induced by SSA can lead to down-regulation of genes important for cell division, including Cyclin A2 and Aurora A kinase providing an explanation for antiproliferative effects of SSA. SF3B1(SAP155) is the direct target of GEX1A [66]. SF3B3 has been shown to be direct interactor of another anti-spliceosome drug-pladienolide B [67]. SSA and meayamycin are among the most potent anticancer drugs that do not bind to either DNA or microtubule [45]. Pladienolide synthetic derivate E7107 has entered phase I clinical trials against thyroid cancer and has led to stable disease or delayed disease progression in a subset of patients [68]. Mechanistically, there is an accumulating evidence for strong link of splicing machinery deregulation, cell cycle progression and genome instability [69, 70, 71, 72]. Nevertheless, a substantial challenge for applications of novel promising anti-spliceosome drugs is identifying subsets of tumors that might be susceptible to splice-inhibition therapy [73]. To our knowledge the current proposal is the first study in the field of breast cancer research which provides a detailed approach to identify such subsets of tumors. In this context, we suggest that for those breast cancer patients who have tumors enriched with deregulated (overexpressed) genes of proteasome and spliceosome, anti-proteasome and anti-spliceosome drugs could be a good alternative to inhibit cell cycle progression and tumor growth. In contrast, potentially, the patients who still would have high recurrence rate, but without deregulated expression pattern of spliceosome genes in their tumors, may not benefit from anti-spliceosome therapy.

More intriguing potential drug for such breast cancer patients would be naturally occurring biflavonoid isoginkgetin which have been shown to be general inhibitor of splicing in vitro and in vivo [50]. In in vitro reactions, isoginkgetin caused the arrest of spliceosome assembly and sequestered pre-mRNA in complex A. Importantly, isoginkgetin is also known as an inhibitor of tumor invasion through regulation of PI3K/Akt/NF-kappa B signaling pathway in MDA-MB-231 breast cancer cell line [74]. As in our study we observed robust upregulation of several genes specific for the following complex B in the “spliceosome-enriched” subtype, isoginkgetin could be an even more specific drug for such breast cancer patients than pladienolides, spliceostatin A and sudemycins [48].

Alternatively, those 27 genes of proteasome and 25 spliseosome genes robustly overexpressed in SAGC HR subgroups could be used directly to develop a specific assay(s) for prognosis of breast cancer outcome. Correct identification of that specific subgroup of patients (either by SAGC or using the genes of proteasome and/or spliceosome as biomarkers or both in combination) would facilitate development of novel systemic treatment schemes and modalities for them. Such schemes would use the combination of conventional drugs targeting cell cycle and DNA replication, hormonotherapy as well as agents targeting specific components of spliceosome.

Another important property of the most anti-spliceosome drugs is their highly selective tumor cytotoxicity as opposed to normal tissues [46, 47]. One could suggest, that transient, short term tumors treatment with drugs specifically targeting spliceosome may not lead to substantial drug side effects, though it could potentially lead to significant increase of tumor's sensitivity in the course of the following standard chemotherapy. On the other hand, efficiency/drug resistance effects of the novel combined treatment schemes could be tested by the SAGC (FIG. 17A-I). Specific trial studies in specific patients subgroups identified by SAGC could provide the clues to resolve that challenge.

### The Clinical Data Used in the Above Experiments

The published datasets as well as our own original breast cancer dataset used in this document are summarized in Table 4.

For the microarray and survival analyses we used two independent microarray datasets from Sweden—the Uppsala cohort representing breast=cancer patients resected in Uppsala County and the Stockholm cohort derived from breast cancer patients operated on at the Karolinska Hospital [22, 75]; one dataset from France-including 250 breast cancer patients at the Institute Paoli-Calmettes and Hopital Nord (Marseille) [76]. The Harvard cohort 1 included primary 38 breast tumors classified as basal-like and non-basal-like subtypes obtained as anonymous samples from Harvard SPORE blood and tissue repository [77]. The Harvard cohort 2 (115 samples) was another collection of primary breast tumors from NCI-Harvard Breast SPORE blood and tissue repository [78]. The Singapore samples were derived from patients operated on at the National University Hospital (Singapore) from Feb. 1, 2000, through Jan. 31, 2002 [22]. Colon cancer microarray dataset was collected in Academic Medical Center in Amsterdam (Netherlands) [43], Non-Small Lung Cancer Dataset—from Erasmus University Medical Center in Rotterdam (Netherlands) [44].

To obtain the additional large testing group used to verify the SAGC as well as to do massive DEG analysis, we combined the microarray expression datasets from 5 independent BC cohorts (Metadata: combined the Oxford, the Guys hospital (GEO accessions: GSE6532, GSE9195), the Harvard (GEO accession: GSE19615), the Marseille (GEO accession: GSE21653) and the BII-OriGene cohorts (GEO accession: GSE61304). To obtain the testing group for verification of the SAGC in G3 breast tumors and other tumors subpopulations we joined microarray expression datasets of the Uppsala and Stockholm cohorts into the whole dataset with consequent batch effect correction using dChip [79]. Further, we checked the quality of the joined dataset applying the R-package arrayQualityMetrics [80].

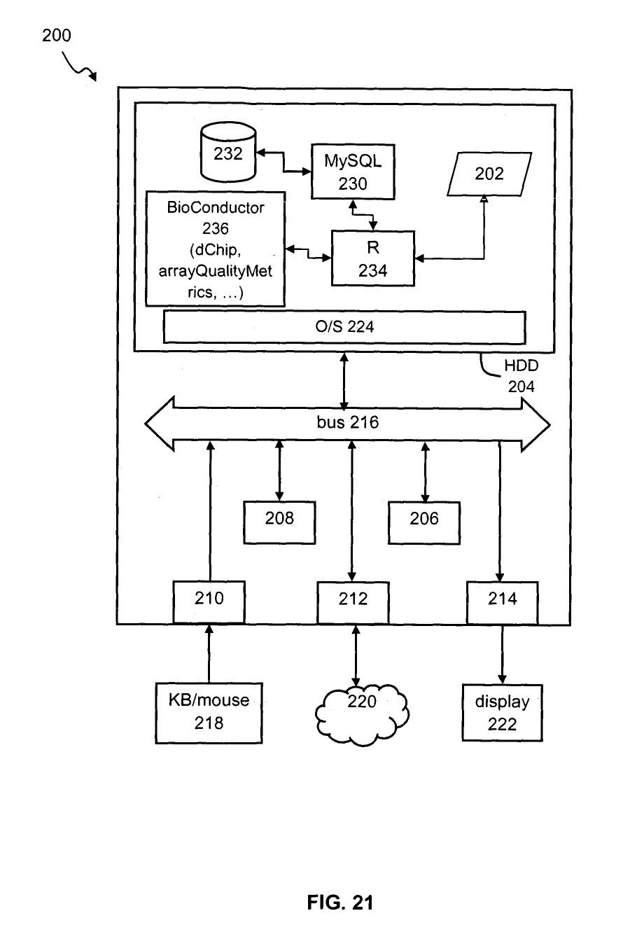

The methods according to the described embodiments may be implemented on a standard computer system such as an Intel IA-32 based computer, as shown in FIG. 21. Some or all of the processes 1 to 25 (FIG. 1 and FIG. 2) executed by the system 200 are implemented in the form of programming instructions of one or more software modules or components 202 stored on tangible and non-volatile (e.g., solid-state or hard disk) storage 204 associated with the computer system 200, as shown in FIG. 21. However, it will be apparent that the processes could alternatively be implemented, either in part or in their entirety, in the form of one or more dedicated hardware components, such as application-specific integrated circuits (ASICs), and/or in the form of configuration data for configurable hardware components such as field programmable gate arrays (FPGAs), for example.

As shown in FIG. 21, the system 200 includes standard computer components, including random access memory (RAM) 206, at least one processor 208, and external interfaces 210, 212, 214, all interconnected by a bus 216. The external interfaces include universal serial bus (USB) interfaces 210, at least one of which is connected to a keyboard 218 and pointing device such as a mouse, and a network interface connector (NIC) 212 which connects the system 200 to a communications network 220 such as the Internet.

The system 200 also includes a display adapter 214, which is connected to a display device such as an LCD panel display 222, and a number of standard software modules, including an operating system 224 such as Linux or Microsoft Windows. The system 200 may include structured query language (SQL) support 230 such as MySQL, available from http://www.mysql.com, which allows data to be stored in and retrieved from an SQL database 232. The database 232 may store the gene expression data from the plurality of subjects, for example, and may also store the output of the processes described above (classification parameters, identification of gene pairs, and so on). In one embodiment, the modules implementing the above processes are realized as scripts 202 received as input by the R statistical programming environment 234, which has associated with it a plurality of add-on modules including dChip and arrayQualityMetrics of Bioconductor 236. The scripts 202 contain instructions for performing, within the R environment 234, a series of computational operations corresponding to some or all of the steps 1 to 25 of FIGS. 1 and 2.

Certain embodiments may relate to a kit for predicting clinical outcome in a subject having a medical condition. The kit may comprise a plurality of polynucleotide sequences or other probes capable of specifically binding to a target sequence in a sample (for example, a tissue sample, or a body fluid sample such as blood, urine, saliva, etc.) to allow a concentration or copy number of the target sequence in the sample to be quantified. As is well-known in the art, such probes may comprise a detectable label such as a fluorescent, phosphorescent or radioactive moiety which emits detectable electromagnetic or other radiation. For example, the probes may be fluorescent reporter probes used in a quantitative PCR process. In another example, the probes may be unlabelled oligonucleotide or cDNA probes bound to a solid support, to which labelled target sequences (each bound to a fluorescent dye, for example) can specifically hybridize in order to quantify the concentration or copy number of the target sequences.

The kit may comprise a plurality of polynucleotide sequences being capable of specifically hybridizing to and/or detecting a gene of a plurality of genes and/or an expression product of the gene to obtain respective gene expression values. In particular, the plurality of genes may comprise genes of one or more of the sense-antisense gene pairs (SAGPs) listed in Table 1A. Preferably the kit comprises polynucleotide sequences corresponding to no more than 100 genes.

The kit may also comprise written instructions for comparing the respective gene expression values to optimal gene expression cut-off values for respective ones of the plurality of genes in order to make the prediction of clinical outcome. For example, the written instructions may contain the cut-off values and an indication of the clinical relevance of expression of respective genes being above or below respective cut-off values.

In some embodiments the kit may comprise, alternatively to or in addition to the written instructions, a tangible computer-readable medium having stored thereon machine-readable instructions for causing a computer processor to compare the respective gene expression values to optimal gene expression cut-off values for respective ones of the plurality of genes in order to make the prediction of clinical outcome. In some embodiments the optimal gene expression cut-off values are determined for each SAGP by:
(i) defining a plurality of trial values for each of two cut-off values cⁱ and c^{j};
(ii) for each of a plurality of angles α, for each subject, and for each of the trial cut-off values cⁱ and c^{j}:
(a) comparing the expression values to a respective pair of lines in a two-dimensional space spanned by the expression values to obtain comparison data indicating on which side of the pair of lines the expression values for the corresponding subject lie, the pair of lines being formed using the cut-off values cⁱ and c^{j}, each of the lines having angle α to a direction in the space indicating increasing values of a corresponding one of the expression values; and
(b) generating at least one SPM based on the comparison data; and
(iii) selecting the one of the SPMs (‘the maximally predictive SPM’) which has the maximal statistical value in predicting the survival times of the subjects,
whereby the cut-off values cⁱ and c^{j} for the maximally predictive SPM are the optimal gene expression cut-off values.

### Advantageous Features of the Invention

Preferred embodiments of the invention exhibit the following advantageous features:

1. A fully automatic method of identification of human breast cancer associated ccSAGPs which expression pattern models and model′ cut-off values form a highly confidence combined survival prognostic signature (CSPS) stratifying the patients onto favorable and unfavorable subgroups predicted within conventional clinical or/and molecular classification systems of breast tumors (FIG. 1, steps 1-6).

2. A fully automatic method of identification of human breast cancer associated ccSAGPs which expression pattern models and model′ cut-off values form a highly confidence CSPS stratifying the patients onto favorable and unfavorable subgroups within conventional clinical or/and molecular classification of colon and lung tumors. The same is applicable to any other oncologic diseases or other disease when information about patient's survival or other time-course treatment response is available.

3. A fully automatic method of breast cancer patient's risk stratification based on statistical voting of negatively and positively correlated and physically interconnected ccSAGPs forming cancer's patient CSPS which stratifying the patients onto favorable and unfavorable clinical subgroups and which is also applicable to the stratification of breast cancer, lung cancer, and colon cancer types or subtypes. The same is applicable to any other oncologic diseases or other disease when information about patient's survival or other time-course treatment response is available.

4. More generally, a fully automatic method of cancer patient's risk stratification based on statistical voting of correlated or co-regulated or physically interconnected gene pairs (or/and other linked feature pairs characterizing neoplastic process) forming cancer patient′ CSPS, which stratifying/discriminating the patients having a given tumor type (or/and a subtype) onto favorable and unfavorable clinical subgroups. The same is applicable to any oncologic diseases or other disease when information about patient's survival or other time-course treatment response is available.

5. A method of implementation of sense-antisense gene classifier (SAGC) as a complex biomarker composed of a specific subset of gene pairs which can substantially improve the accuracy of re-classification of breast cancer tumors into relatively low-risk (unfavorable) and relatively high-risk (favorable) subgroups within patient's group defined by conventional clinical or/and molecular classification system of breast tumors (FIG. 2). SAGC may be also implemented not only to breast tumors, but also to any oncologic diseases or other disease when information about patient's survival or other time-course treatment response is available.

6. A fully automatic method of patient's survival prediction adapted to any correlated gene pairs (including ccSAGPs and all other subclasses of sense-antisense transcripts and gene pairs) and termed the 2-D rotation data-driven grouping (2-D RDDg). The method is applicable not only to ccSAGPs, but also to any significantly correlated gene pairs/transcripts including other known classes of sense-antisense gene pairs and sense-antisense transcripts pairs.

7. A computerized method of integration of survival information for individual gene pairs into a dramatically improved patients partition which is based on statistically weighted voting grouping procedure. The method is applicable not only to individual gene pairs but also to any individual genes or to other characteristics of the patients with available survival information.

8. A computerized method for implication of any gene pairs including sense-antisense gene pairs for prognosis/prediction and stratification in cancer patients with available survival information. The method includes estimation of the optimal cut-offs for expression values for each of the two genes, the optimal design and rotation angle using 2-D RDDg procedure in one training cohort composed of at least 50 breast cancer patients with consequent testing using 2-D RDDg procedure in at least one cohort composed of at least 50 patients. The method is applicable not only to breast cancer patients, but also to any cancer patients with available survival information.

9. A computerized method for implication of sense-antisense gene classifier which includes at least two steps (training and testing procedures) using 2-D RDDg procedure coupled with WVG procedure and is based on methods in features 5 and 4 (FIG. 2, Steps 7.1 and 7.2). The method includes estimation of the optimal parameters for 2-D RDDg procedure (training procedure) for individual gene pairs and their testing using 2-D RDDg procedure as in claim 8. The method is applicable not only to breast cancer patients, but also to any cancer patients with available survival information.

10. A computerized method for stratification and prediction of clinical outcome of ER“+”, LN“−” breast cancer patients who received adjuvant systemic tamoxifen treatment after curative surgery using the RNF139/TATDN1 SAGP. The method includes estimation of the optimal parameters for 2-D RDDg procedure (training procedure) for the individual gene pair and its testing using 2-D RDDg procedure as in claim 8.

11. A computerized method for stratification and prediction of clinical outcome of ER“+”, LN“−” breast cancer patients received adjuvant systemic tamoxifen treatment after curative surgery using SAGC. The method includes estimation of the optimal parameters for 2-D RDDg procedure (training procedure) and the testing procedure for all ccSAGPs comprising SAGC as described in feature 8. SAGC is implemented as in feature 9.

12. A computerized method for stratification and prognosis of clinical outcome of breast cancer patients with grade 3 tumors using the VPRBP/RBM15B SAGP as well as the full SAGC. The method includes estimation of the optimal parameters for 2-D RDDg procedure (training procedure) and the testing procedure for all ccSAGPs comprising SAGC as described in feature 8. SAGC is implemented as in feature 9.

13. A computerized method for stratification and prognosis of clinical outcome of breast cancer patients with grade 3 and grade 3-like tumors using the SAGPs C18orf8/NPC1 and EME1/LRRC59 as well as the full SAGC. The method includes estimation of the optimal parameters for 2-D RDDg procedure (training procedure) and the testing procedure for all ccSAGPs comprising SAGC as described in feature 8. SAGC is implemented as in feature 9.

14. A computerized method for stratification and prognosis of clinical outcome of breast cancer patients with grade 1 and grade 1-like tumors using the SHMT1/SMCR8 SAGP as well as the full SAGC. The method includes estimation of the optimal parameters for 2-D RDDg procedure (training procedure) and the testing procedure for all ccSAGPs comprising SAGC as described in feature 8. SAGC is implemented as in feature 9.

15. A computerized method for stratification and prognosis of clinical outcome of breast cancer patients with grade 1 breast tumors using the full SAGC. The method includes estimation of the optimal parameters for 2-D RDDg procedure (training procedure) and the testing procedure for all ccSAGPs comprising SAGC as described in feature 8. SAGC is implemented as in feature 9.

16. A computerized method for stratification and prognosis of clinical outcome of ER“−” breast cancer patients from total unselected groups using the CTNS/TAX1 BP3 SAGP as well as the full SAGC. The method includes estimation of the optimal parameters for. 2-D RDDg procedure (training procedure) and the testing procedure for all ccSAGPs comprising SAGC as described in feature 8. SAGC is implemented as in feature 9.

17. A computerized method for stratification and prognosis of clinical outcome of breast cancer patients with basal-like grade 3 (G3) breast tumors using the SAGPs CTNS/TAX1BP3 and RNF139/TATDN1 as well as the full SAGC. The method includes estimation of the optimal parameters for 2-D RDDg procedure (training procedure) and the testing procedure for all ccSAGPs comprising SAGC as described in feature 8. SAGC is implemented as in feature 9.

18. A computerized method for stratification and prognosis of clinical outcome of breast cancer patients with Luminal A breast tumors using the BIVM/KDELC1 SAGPs as well as the full SAGC. The method includes estimation of the optimal parameters for 2-D RDDg procedure (training procedure) and the testing procedure for all ccSAGPs comprising SAGC as described in feature 8. SAGC is implemented as in feature 9.

19. A computerized method for stratification and prognosis of clinical outcome of ER“+”, LN“−”, PgR“+” breast cancer patients with breast tumors <=2 cm on the moment of curative surgery who usually do not receive any systemic treatment, using the SAGC. The method includes estimation of the optimal parameters for 2-D RDDg procedure (training procedure) and the testing procedure for all ccSAGPs comprising SAGC as described in feature 8. SAGC is implemented as in feature 9.

20. A computerized method for stratification and prognosis of clinical outcome of colon cancer patients with stage II tumors using the SAGC. The method includes estimation of the optimal parameters for 2-D RDDg procedure (training procedure) and the testing procedure for all ccSAGPs comprising SAGC as described in feature 8 SAGC is implemented as in feature 9.

21. A computerized method for stratification and prognosis of clinical outcome of non-small lung cancer patients from total unselected group using the SAGC. The method includes estimation of the optimal parameters for 2-D RDDg procedure (training procedure) and the testing procedure for all ccSAGPs comprising SAGC as described in feature 8. SAGC is implemented as in feature 9.

22. A computerized method for identification of novel biomarkers of breast tumors heterogeneity as well as novel potential candidates for drug targets using SAGC. i) stratification of breast cancer patients into low-risk and high-risk subgroups using the workflow described in steps 3-6 of FIG. 1 and in step 7 of FIG. 2; ii) identification of robust differentially expressed genes between the subgroups in each unrelated cohort; iii) intersection of the lists of differentially expressed genes among several unrelated breast cancer cohorts; iv) identification of overrepresented gene ontology terms for the list of intersection. Method is applicable not only to breast cancer patients, but also to any cancer patients or disease patients with available survival information.

23. A computerized method for the identification of a high risk disease recurrence patient subgroup of BC patients, which primary tumors are characterized by over-expression of “proteasome-enriched” and “spliceosome-enriched” genes (Table 10) including the genes differentially expressed between low-risk and high-risk groups defined by SAGC in several original patient cohorts. Such specific patient subgroups are characterized by: i) significantly higher rate of distant metastases/distant recurrence events; ii) more often resistance against primary chemotherapy and hormone therapy (FIGS. 17C, F and I); iii) significant enrichment by genes belonging to the proteasome and spliceosome (Tables 10 and 11, FIG. 17)). Method includes all features of claim 1 and provides an implementation of the SAGC in computational procedures on the steps 3-6 from FIG. 1 of the current invention.

24. A computerized method for the stratification of BC patients and an identification of a high-risk subgroup of the patients with “spliceosome-enriched” in total unselected groups of the patients using 27-gene prognostic signature (or proteasome-based predictor) of proteasome machinery and 25-gene prognostic signature of spliceosome machinery (or spliceosome-based predictor) (Table 10).

25. An assay/kit for detecting multidrug-resistant tumors (i.e., resistant to chemotherapy- and hormonotherapy) in breast tumors and their treatment monitoring using the proteasome-based predictor and spliceosome-based predictor of (Table 10).

26. A method for identification of novel drug targets using strategy of discovery of SAGC classifier and the signature of spliceosome complex B.

27. A method for identification of novel cancer biomarker or drug targets using genes of SAGC or the products derived from the genes of that molecular signature and used as the biomarkers or drug targets.

28. A method for identification of novel cancer biomarker or drug targets using genes of the proteasome and spliceosome or the products derived from the same gene's and used as the biomarkers or drug targets.

29. An assay/kit using combined any genes of SAGC and their products as biomarkers of breast, lung, colon and other cancers.

TABLE 1A
Breast cancer-relevant SAGPs identified in embodiments of the current invention. Highlighted (bold text) BCR-SAGPs comprise
SAGC. *: http://mgc.nci.nih.gov/

| | | | | Mammalian | | | Length of |
|---|---|---|---|---|---|---|---|
| # of | # of | | | Gene | | | sense- |
| BCR- | Host | Host Gene | Representative | Collection | Host gene description (UCSC Genome | DNA | antisense |
| SAGP | Gene | Symbol | transcript ID | cDNA clone ID* | Browser) | strand | overlap, bp |
| 1 | 1 | MSH6 | NM_000179 | BC004246 | mutS homolog 6 | “+” | 34 |
| | 2 | FBXO11 | NM_025133 | BC130445 | F-box only protein 11 isoform 1 | “−” | |
| 2 | 3 | BOLA2 | NM_001031827 | BC130401 | bolA homolog 2 (E. coli) (BOLA2) | “−” | 464 |
| | 4 | SLX1A | NM_001014999 | BC141497 | SLX1 structure-specific endonuclease | “+” | |
| | | | | | subunit homolog A (S. cerevisiae) | | |
| 3 | 5 | C6orf120 | NM_001029863 | BC051700 | hypothetical protein LOC387263 | “+” | 2402 |
| | 6 | PHF10 | NM_133325 | BC020954 | PHD finger protein 10 isoform b | “−” | |
| 4 | 7 | FBXO8 | NM_012180 | BC014679 | F-box only protein 8 | “−” | 575 |
| | 8 | KIAA1712 | NM_001040157 | BC038667 | HBV PreS1-transactivated protein 3 | “+” | |
| | | | | | isoform a | | |
| 5 | 9 | C11orf48 | NM_024099 | BC001434 | hypothetical protein LOC79081 | “−” | 116 |
| | 10 | C11orf83 | NM_001085372 | BC090057 | hypothetical protein LOC790955 | “+” | |
| 6 | 11 | POLR2I | NM_006233 | BC067794 | DNA directed RNA polymerase II | “−” | 319 |
| | | | | | polypeptide I | | |
| | 12 | TBCB | NM_001281 | BC005969 | cytoskeleton associated protein 1 | “+” | |
| 7 | 13 | ATP1B1 | NM_001677 | BC000006 | ATPase, Na+/K+ transporting, beta 1 | “+” | 192 |
| | | | | | polypeptide (ATP1B1) | | |
| | 14 | NME7 | NM_013330 | BC006983 | nucleoside-diphosphate kinase 7 isoform a | “−” | |
| 8 | 15 | WDR77 | NM_024102 | BC001679 | WD repeat domain 77 | “−” | 88 |
| | 16 | ATP5F1 | NM_001688 | BC005366 | ATP synthase, H+ transporting, | “+” | |
| | | | | | mitochondrial F0 | | |
| 9 | 17 | SSB | NM_003142 | BC001289 | autoantigen La | “+” | 308 |
| | 18 | METTL5 | NM_014168 | BC093014 | methyltransferase like 5 | “−” | |
| 10 | 19 | NR2C2 | NM_003298 | BC030715 | nuclear receptor subfamily 2, group C, | “+” | 762 |
| | | | | | member 2 | | |
| | 20 | MRPS25 | NM_022497 | BC003590 | mitochondrial ribosomal protein S25 | “−” | |
| 11 | 21 | ANXA9 | NM_003568 | BC005830 | annexin A9 | “+” | 566 |
| | 22 | FAM63A | NM_018379 | BC032321 | hypothetical protein LOC55793 isoform 1 | “−” | |
| 12 | 23 | KMO | NM_003679 | BC005297 | kynurenine 3-monooxygenase | “+” | 2493 |
| | 24 | OPN3 | NM_014322 | BC036773 | opsin 3 | “−” | |
| 13 | 25 | RFXANK | NM_003721 | BC114563 | regulatory factor X-associated ankyrin- | “+” | 455 |
| | | | | | containing protein | | |
| | 26 | NR2C2AP | NM_176880 | BC057837 | TR4 orphan receptor associated protein | “−” | |
| | | | | | TRA16 | | |
| 14 | 27 | EIF3J | NM_003758 | BC002719 | eukaryotic translation initiation factor 3, | “+” | 108 |
| | 28 | SPG11 | NM_025137 | BC024161 | spastic paraplegia 11 (autosomal | “−” | |
| | | | | | recessive) | | |
| 15 | 29 | BUD31 | NM_003910 | BC104670 | G10 protein | “+” | 2878 |
| | 30 | PTCD1 | NM_015545 | BC103661 | pentatricopeptide repeat domain 1 | “−” | |
| 16 | 31 | ESRRA | NM_004451 | BC011528 | estrogen-related receptor alpha | “+” | 46 |
| | 32 | TRMT112 | NM_016404 | BC017172 | hypothetical protein LOC51504 | “−” | |
| 17 | 33 | PIGB | NM_004855 | BC017711 | phosphatidylinositol glycan, class B | “+” | 426 |
| | 34 | CCPG1 | NM_004748 | BC015203 | cell cycle progression 1 (CCPG1) | “−” | |
| 18 | 35 | CTNS | NM_004937 | BC032850 | cystinosis, nephropathic isoform 2 | “+” | 211 |
| | 36 | TAX1BP3 | NM_014604 | BC023980 | Tax1 (human T-cell leukemia virus | “−” | |
| | | | | | type I) | | |
| 19 | 37 | PLA2G4B | NM_005090 | BC013415 | JMJD7-PLA2G4B protein | “+” | 2 |
| | 38 | SPTBN5 | NM_016642 | NA | spectrin, beta, non-erythrocytic 5 | “−” | |
| 20 | 39 | TOM1L1 | NM_005486 | BC029396 | target of myb1-like 1 | “−” | 762 |
| | 40 | COX11 | NM_004375 | BC005895 | COX11 homolog | “+” | |
| 21 | 41 | TADA3L | NM_006354 | BC013433 | transcriptional adaptor 3-like isoform a | “+” | 189 |
| | 42 | ARPC4 | NM_005718 | BC065423 | actin related protein 2/3 complex subunit 4 | “−” | |
| 22 | 43 | STUB1 | NM_005861 | BC017178 | STIP1 homology and U-box containing | “−” | 1102 |
| | | | | | protein 1 | | |
| | 44 | JMJD8 | NM_001005920 | BC137101 | jumonji domain containing 8 | “+” | |
| 23 | 45 | ACAT2 | NM_005891 | BC000408 | acetyl-Coenzyme A acetyltransferase 2 | “−” | 558 |
| | 46 | TCP1 | NM_001008897 | BC000665 | T-complex protein 1 isoform b | “+” | |
| 24 | 47 | NUDT5 | NM_014142 | BC000025 | nudix-type motif 5 | “−” | 183 |
| | 48 | CDC123 | NM_006023 | BC009598 | cell division cycle 123 | “+” | |
| 25 | 49 | AIMP2 | NM_006303 | BC002853 | aminoacyl tRNA synthetase complex- | “+” | 1588 |
| | 50 | EIF2AK1 | NM_014413 | BC006524 | interacting multifunctional protein 2 | “−” | |
| | | | | | eukaryotic translation initiation factor | | |
| | | | | | 2-alpha | | |
| 26 | 51 | SLC25A40 | NM_018843 | BC027322 | mitochondrial carrier family protein | “−” | 149 |
| | 52 | DBF4 | NM_006716 | BC047693 | activator of S phase kinase | “+” | |
| 27 | 53 | NPRL2 | NM_006545 | BC050412 | tumor suppressor candidate 4 | “+” | 190 |
| | 54 | CYB561D2 | NM_007022 | BC047691 | cytochrome b-561 domain containing 2 | “−” | |
| 28 | 55 | RER1 | NM_007033 | BC004965 | RER1 retention in endoplasmic reticulum 1 | “+” | 643 |
| | 56 | PEX10 | NM_002617 | BC018198 | peroxisome biogenesis factor 10 isoform 2 | “−” | |
| 29 | 57 | RNF139 | NM_007218 | BC021571 | ring finger protein 139 | “+” | 125 |
| | 58 | TATDN1 | NM_032026 | BC064964 | TatD DNase domain containing 1 | “−” | |
| 30 | 59 | RHOQ | NM_012249 | BC056154 | ras-like protein TC10 | “+” | 3415 |
| | 60 | PIGF | NM_002643 | BC029408 | phosphatidylinositol glycan anchor | “−” | |
| | | | | | biosynthesis, | | |
| 31 | 61 | AUP1 | NM_181575 | BC033646 | ancient ubiquitous protein 1 | “+” | 443 |
| | 62 | HTRA2 | NM_013247 | BC000096 | HtrA serine peptidase 2 isoform 1 | “−” | |
| | | | | | preproprotein | | |
| 32 | 63 | RBM15B | NM_013286 | BC001367 | RNA binding motif protein 15B | “+” | 2039 |
| | 64 | VPRBP | NM_014703 | BC022792 | HIV-1 Vpr binding protein | “−” | |
| 33 | 65 | C18orf8 | NM_013326 | BC008305 | colon cancer-associated protein Mic1 | “+” | 282 |
| | 66 | NPC1 | NM_000271 | BC063302 | Niemann-Pick disease, type C1 | “−” | |
| | | | | | precursor | | |
| 34 | 67 | GLT8D1 | NM_018446 | BC119670 | glycosyltransferase 8 domain containing 1 | “+” | 192 |
| | 68 | SPCS1 | NM_014041 | BC000884 | signal peptidase complex subunit 1 | “−” | |
| | | | | | homolog | | |
| 35 | 69 | MKRN2 | NM_014160 | BC001799 | makorin ring finger protein 2 | “+” | 111 |
| | 70 | RAF1 | NM_002880 | BC018119 | v-raf-1 murine leukemia viral oncogene | “−” | |
| | | | | | homolog | | |
| 36 | 71 | C1orf131 | NM_152379 | BC036800 | hypothetical protein LOC128061 | “+” | 6 |
| | 72 | GNPAT | NM_014236 | BC000450 | glyceronephosphate O-acyltransferase | “−” | |
| 37 | 73 | PDSS1 | NM_014317 | BC049211 | prenyl diphosphate synthase, subunit 1 | “+” | 202 |
| | 74 | ABI1 | NM_001012751 | BC024254 | abl-interactor 1 isoform c | “−” | |
| 38 | 75 | PPIL2 | NM_014337 | BC000022 | peptidylprolyl isomerase-like 2 isoform a | “+” | 377 |
| | 76 | YPEL1 | NM_013313 | BC034486 | yippee-like 1 | “−” | |
| 39 | 77 | TMEM97 | NM_014573 | BC017362 | transmembrane protein 97 | “−” | 359 |
| | 78 | IFT20 | NM_174887 | BC038094 | intraflagellar transport protein IFT20 | “+” | |
| 40 | 79 | KIAA0195 | NM_014738 | BC042942 | hypothetical protein LOC9772 | “+” | 192 |
| | 80 | CASKIN2 | NM_020753 | BC066643 | cask-interacting protein 2 isoform a | “−” | |
| 41 | 81 | MRPL19 | NM_014763 | BC021051 | mitochondrial ribosomal protein L19 | “+” | 41 |
| | 82 | C2orf3 | NM_003203 | BC064559 | hypothetical protein LOC6936 | “−” | |
| 42 | 83 | TOX4 | NM_014828 | BC020727 | epidermal Langerhans cell protein LCP1 | “−” | 1038 |
| | 84 | METTL3 | NM_019852 | BC001650 | methyltransferase like 3 | “+” | |
| 43 | 85 | CEP68 | NM_015147 | BC030534 | centrosomal protein 68 kDa | “+” | 155 |
| | 86 | RAB1A | NM_004161 | BC000905 | RAB1A, member RAS oncogene family | “−” | |
| | | | | | isoform 1 | | |
| 44 | 87 | SLC25A32 | NM_030780 | BC021893 | solute carrier family 25, member 32 | “−” | 527 |
| | 88 | DCAF13 | NM_015420 | BC035190 | WD repeats and SOF1 domain | “+” | |
| | | | | | containing | | |
| 45 | 89 | MRPS7 | NM_015971 | BC000241 | mitochondrial ribosomal protein S7 | “−” | 147 |
| | 90 | MIF4GD | NM_020679 | BC033759 | MIF4G domain containing | “+” | |
| 46 | 91 | MRPS18C | NM_016067 | BC005186 | mitochondrial ribosomal protein S18C | “+” | 834 |
| | 92 | FAM175A | NM_139076 | BC016905 | coiled-coil domain containing 98 | “−” | |
| 47 | 93 | KDELC1 | NM_024089 | BC001297 | KDEL (Lys-Asp-Glu-Leu) containing 1 | “+” | 6 |
| | 94 | BIVM | NM_017693 | BC075084 | basic, immunoglobulin-like variable | “−” | |
| | | | | | motif | | |
| 48 | 95 | WDR55 | NM_017706 | BC002482 | WD repeat domain 55 | “+” | 173 |
| | 96 | DND1 | NM_194249 | BC033496 | dead end homolog 1 | “−” | |
| 49 | 97 | ZNF721 | NM_133474 | NA | zinc finger protein 721 | “−” | 454 |
| | 98 | PIGG | NM_017733 | BC001249 | phosphatidylinositol glycan anchor | “+” | |
| | | | | | biosynthesis, class G | | |
| 50 | 99 | INTS8 | NM_017864 | BC099915 | integrator complex subunit 8 | “+” | 269 |
| | 100 | CCNE2 | NM_057749 | BC007015 | cyclin E2 | “−” | |
| 51 | 101 | GLOD4 | NM_016080 | BC015848 | glyoxalase domain containing 4 | “+” | 59 |
| | 102 | RNMTL1 | NM_018146 | BC050614 | RNA methyltransferase like 1 | “−” | |
| 52 | 103 | SUPT7L | NM_014860 | BC045638 | SPTF-associated factor 65 gamma | “−” | 112 |
| | 104 | SLC4A1AP | NM_018158 | BC099711 | solute carrier family 4 (anion exchanger), | “+” | |
| 53 | 105 | C11orf57 | NM_018195 | BC048133 | hypothetical protein LOC55216 isoform a | “+” | 339 |
| | 106 | TIMM8B | NM_012459 | BC000711 | translocase of inner mitochondrial | “−” | |
| | | | | | membrane 8 | | |
| 54 | 107 | COG1 | NM_018714 | BC047465 | component of oligomeric golgi complex 1 | “−” | 1154 |
| | 108 | FAM104A | NM_001098832 | BC025238 | hypothetical protein LOC84923 isoform 1 | “+” | |
| 55 | 109 | TNFAIP1 | NM_021137 | BC001949 | tumor necrosis factor, alpha-induced | “+” | 377 |
| | | | | | protein 1 | | |
| | 110 | POLDIP2 | NM_015584 | BC009265 | DNA polymerase delta interacting protein 2 | “−” | |
| 56 | 111 | FAM173A | NM_023933 | BC001181 | hypothetical protein LOC65990 | “+” | 9 |
| | 112 | CCDC78 | NM_001031737 | BC027941 | coiled-coil domain containing 78 | “−” | |
| 57 | 113 | C13orf34 | NM_024808 | BC056143 | aurora borealis | “+” | 789 |
| | 114 | DIS3 | NM_014953 | BC012468 | DIS3 mitotic control isoform b | “−” | |
| 58 | 115 | PYROXD1 | NM_024854 | BC021662 | pyridine nucleotide-disulphide | “+” | 2339 |
| | | | | | oxidoreductase | | |
| | 116 | RECQL | NM_002907 | BC001052 | RecQ protein-like | “−” | |
| 59 | 117 | RPF1 | NM_025065 | BC016051 | RNA processing factor 1 | “+” | 28 |
| | 118 | GNG5 | NM_005274 | BC003563 | guanine nucleotide binding protein (G | “−” | |
| | | | | | protein), | | |
| 60 | 119 | ITPKC | NM_025194 | BC026903 | inositol 1,4,5-trisphosphate 3-kinase C | “+” | 5 |
| | 120 | C19orf54 | NM_198476 | BC020262 | hypothetical protein LOC284325 | “−” | |
| 61 | 121 | MAK16 | NM_032509 | BC028230 | MAK16 homolog | “+” | 2543 |
| | 122 | C8orf41 | NM_001102401 | BC007387 | hypothetical protein LOC80185 | “−” | |
| 62 | 123 | POLR2C | NM_032940 | BC000409 | DNA directed RNA polymerase II | “−” | 52 |
| | 124 | DOK4 | NM_018110 | BC001540 | polypeptide C | “+” | |
| | | | | | docking protein 4 | | |
| 63 | 125 | BCCIP | NM_078468 | BC009771 | BRCA2 and CDKN1A-interacting protein | “+” | 216 |
| | | | | | isoform | | |
| | 126 | DHX32 | NM_018180 | BC002473 | DEAD/H (Asp-Glu-Ala-Asp/His) box | “−” | |
| | | | | | polypeptide 32 | | |
| 64 | 127 | SMCR8 | NM_144775 | BC014179 | Smith-Magenis syndrome | “+” | 184 |
| | 128 | SHMT1 | NM_148918 | BC007979 | chromosome region, | “−” | |
| | | | | | serine hydroxymethyltransferase 1 | | |
| | | | | | (soluble) | | |
| 65 | 129 | KRT10 | NM_000421 | BC034697 | keratin 10 | “−” | 3490 |
| | 130 | TMEM99 | NM_145274 | BC015365 | transmembrane protein 99 | “+” | |
| 66 | 131 | ARRDC1 | NM_152285 | BC032346 | arrestin domain containing 1 | “+” | 29 |
| | 132 | C9orf37 | NM_032937 | BC011781 | chromosome 9 open reading frame 37 | “−” | |
| 67 | 133 | EME1 | NM_152463 | BC016470 | essential meiotic endonuclease 1 | “−” | 227 |
| | 134 | LRRC59 | NM_018509 | BC017168 | homolog 1 | “+” | |
| | | | | | leucine rich repeat containing 59 | | |
| 68 | 135 | PUSL1 | NM_153339 | BC034304 | pseudouridylate synthase-like 1 | “+” | 93 |
| | 136 | CPSF3L | NM_017871 | BC013904 | cleavage and polyadenylation specific | “−” | |
| | | | | | factor | | |
| 69 | 137 | MLX | NM_170607 | BC010689 | transcription factor-like protein 4 isoform | “−” | 893 |
| | 138 | PSMC3IP | NM_016556 | BC008792 | PSMC3 interacting protein isoform 2 | “+” | |
| 70 | 139 | ADCK5 | NM_174922 | BC032402 | aarF domain containing kinase 5 | “−” | 8 |
| | 140 | CPSF1 | NM_013291 | BC017232 | cleavage and polyadenylation specific | “+” | |
| | | | | | factor 1, | | |
| 71 | 141 | FAM71E1 | NM_138411 | BC004941 | hypothetical protein LOC112703 | “+” | 268 |
| | 142 | C19orf63 | NM_175063 | BC062607 | hematopoietic signal peptide-containing | “−” | |
| | | | | | isoform | | |
| 72 | 143 | EXOSC8 | NM_181503 | BC020773 | exosome component 8 | “+” | 301 |
| | 144 | FAM48A | NM_017569 | BC030686 | family with sequence similarity 48, | “−” | |
| | | | | | member A | | |
| 73 | 145 | COG5 | NM_006348 | BC068540 | component of oligomeric golgi complex 5 | “+” | 528 |
| | | | | | isoform | | |
| | 146 | DUS4L | NM_181581 | BC111774 | dihydrouridine synthase 4-like | “−” | |

TABLE 1B
Host genes, Affymetrix probe sets and representative RNA transcripts for SAGC. *: http://mgc.nci.nih.gov/

| | | | | | Mammalian | | |
|---|---|---|---|---|---|---|---|
| | | | | | Gene | | |
| | | | | | Collection | | |
| #ccSAGP | Host gene | Affymetrix | | | cDNA clone | Host gene description (UCSC | |
| in the SAGC | symbol | probeset ID | Best RNA ID¹ | DNA strand | ID* | genomic browser) | Cytoband |
| 1 | C18orf8 | B.232348_at | CA395475¹ | − | BC008305 | Colon cancer-associated protein Mic1 | 18q11.2 |
| | NPC1 | A.202679_at | NM_000271 | − | BC063302 | Niemann-Pick disease, type C1 | |
| | | | | | | precursor | |
| 2 | BORA | A.219544_at | NM_024808 | + | BC056143 | Bora, aurora kinase A activator | 13q22.1 |
| | DIS3 | A.218362_s_at | NM_001128226 | − | BC038101 | DIS3 mitotic control homolog (S. cerevisiae) | |
| 3 | AIMP2 | A.209971_x_at | NM_006303 | + | BC002853 | Aminoacyl tRNA synthetase complex- | 7p22 |
| | | | | | | interacting multifunctional protein 2 | |
| | EIF2AK1 | A.217736_s_at | NM_014413 | − | BC010156 | Eukaryotic translation initiation factor | |
| | | | | | | 2-alpha kinase 1 | |
| 4 | SHMT1 | A.217304_at | Y14488² | − | BC022874 | Serine hydroxymethyltransferase 1 | 17p11.2 |
| | | | | | | (soluble) | |
| | SMCR8 | B.227304_at | NM_144775 | + | BC001018 | Smith-Magenis syndrome | |
| | | | | | | chromosome region | |
| 5 | DOK4 | A.209690_s_at | NM_018110 | − | BC001540 | Docking protein 4 | 16q21 |
| | POLR2C | A.208996_s_at | NM_032940 | + | BC028157 | DNA directed RNA polymerase II | |
| | | | | | | polypeptide C | |
| 6 | MRPS18C | B.228019_s_at | NM_016067 | + | BC005186 | Mitochondrial ribosomal protein S18C | 4q21.23 |
| | FAM175A | B.226521_s_at | NM_139076 | − | BC039573 | Family with sequence similarity 175 | |
| 7 | CTNS | A.204925_at | NM_001031681 | + | BC032850 | Cystinosin, lysosomal cystine | 17p13 |
| | | | | | | transporter | |
| | TAX1BP3 | A.209154_at | NM_014604 | − | BC023980 | Tax1 (human T-cell leukemia virus | |
| | | | | | | type I) binding protein 3 | |
| 8 | EME1 | B.234464_s_at | NM_001166131 | + | BC016470 | Essential meiotic endonuclease 1 | 17q21.33 |
| | | | | | | homolog 1 | |
| | LRRC59 | B.234812_at | DA4891773³ | − | BC017168 | Leucine rich repeat containing 59 | |
| 9 | VPRBP | B.226481_at | NM_014703 | − | BC022792 | Vpr (HIV-1) binding protein (VPRBP) | 3p21.2 |
| | RBM15B | A.202689_at | NM_013286 | + | BC139836 | RNA binding motif protein 15B | |
| 10 | RNF139 | A.209510_at | NM_007218 | + | BC064636 | Ring finger protein 139 | 8q24.13 |
| | TATDN1 | B.223231_at | NM_001146160 | − | BC064964 | TatD DNase domain containing 1 | |
| 11 | SSB | A.201139_s_at | NM_003142 | + | BC020818 | Sjogren syndrome antigen B | 2q31.1 |
| | METTL5 | A.221570_s_at | NM_014168 | − | BC000921 | Methyltransferase like 5 | |
| 12 | BIVM | B.222761_at | NM_001159596 | + | BC051813 | Basic, immunoglobulin-like variable | 13q33.1 |
| | | | | | | motif | |
| | KDELC1 | A.219479_at | NM_024089 | − | BC001297 | KDEL (Lys-Asp-Glu-Leu) containing 1 | |

TABLE 2
Patient's grouping and statistical significance levels of the selected pairs (predictors) in two patient cohorts

| | | | | Stockholm cohort | | | | |
|---|---|---|---|---|---|---|---|---|
| | | | | | | 2-D | | |
| | | | | 1-D | 1-D | RDDg | | |
| Affyprobeset | Gene | Affyprobeset | Gene | DDg | DDg | p- | LR | HR |
| for gene 1 | symbol 1 | for gene 2 | symbol 2 | pvalue1 | pvalue2 | value | subgroup | subgroup |
| B.232348_at | C18orf8 | A.202679_at | NPC1 | 3.9E−02 | 1.6E−02 | 1.3E−02 | 151 | 8 |
| A.219544_at | BORA | A.218362_s_at | DIS3 | 8.0E−03 | 3.0E−04 | 2.7E−07 | 137 | 22 |
| A.209971_x_at | AIMP2 | A.217736_s_at | EIF2AK1 | 1.6E−06 | 2.5E−04 | 1.1E−06 | 108 | 51 |
| A.217304_at | SHMT1 | B.227304_at | SMCR8 | 3.8E−02 | 1.3E−02 | 1.3E−03 | 140 | 19 |
| A.209690_s_at | DOK4 | A.208996_s_at | POLR2C | 1.6E−02 | 1.9E−05 | 1.1E−06 | 133 | 26 |
| B.228019_s_at | MRPS18C | B.226521_s_at | FAM175A | 2.3E−04 | 1.1E−01 | 2.3E−05 | 105 | 54 |
| A.204925_at | CTNS | A.209154_at | TAX1BP3 | 3.1E−01 | 5.2E−03 | 4.7E−03 | 138 | 21 |
| B.234464_s_at | EME1 | B.234812_at | LRRC59 | 2.4E−03 | 1.2E−01 | 4.2E−04 | 93 | 66 |
| B.226481_at | VPRBP | A.202689_at | RBM15B | 1.4E−02 | 3.9E−02 | 8.4E−03 | 120 | 39 |
| A.209510_at | RNF139 | B.223231_at | TATDN1 | 7.0E−03 | 9.4E−03 | 6.6E−03 | 140 | 19 |
| A.201139_s_at | SSB | A.221570_s_at | METTL5 | 9.0E−02 | 2.5E−02 | 1.1E−02 | 121 | 38 |
| B.222761_at | BIVM | A.219479_at | KDELC1 | 6.0E−02 | 1.1E−01 | 1.4E−02 | 97 | 62 |
| | | | | | | WVG | | |
| | | | | | | p-value | | |
| | | | | | | 9.58E−10 | 113 | 46 |
| | | | | Uppsala cohort | | | | |
| | | | | 1-D | 1-D | 2-D | | |
| | | | | DDg | DDg | RDDg | | |
| Affyprobeset | Gene | Affyprobeset | Gene | p- | p- | p- | LR | HR |
| for gene 1 | symbol 1 | for gene 2 | symbol 2 | value1 | value2 | value | subgroup | subgroup |
| B.232348_at | C18orf8 | A.202679_at | NPC1 | 3.2E−02 | 2.8E−03 | 5.8E−10 | 240 | 9 |
| A.219544_at | BORA | A.218362_s_at | DIS3 | 3.0E−03 | 1.4E−01 | 1.5E−03 | 218 | 31 |
| A.209971_x_at | AIMP2 | A.217736_s_at | EIF2AK1 | 5.5E−03 | 2.7E−02 | 3.0E−03 | 178 | 71 |
| A.217304_at | SHMT1 | B.227304_at | SMCR8 | 6.1E−02 | 1.0E−02 | 7.1E−06 | 226 | 23 |
| A.209690_s_at | DOK4 | A.208996_s_at | POLR2C | 4.5E−02 | 5.1E−02 | 1.6E−02 | 211 | 38 |
| B.228019_s_at | MRPS18C | B.226521_s_at | FAM175A | 7.1E−02 | 2.9E−02 | 2.3E−03 | 151 | 98 |
| A.204925_at | CTNS | A.209154_at | TAX1BP3 | 2.3E−02 | 2.2E−03 | 2.6E−05 | 233 | 16 |
| B.234464_s_at | EME1 | B.234812_at | LRRC59 | 2.1E−03 | 6.7E−02 | 5.4E−04 | 135 | 114 |
| B.226481_at | VPRBP | A.202689_at | RBM15B | 3.0E−03 | 3.4E−02 | 2.7E−04 | 193 | 56 |
| A.209510_at | RNF139 | B.223231_at | TATDN1 | 4.1E−02 | 1.9E−02 | 4.2E−03 | 226 | 23 |
| A.201139_s_at | SSB | A.221570_s_at | METTL5 | 2.1E−02 | 1.3E−02 | 9.0E−03 | 173 | 76 |
| B.222761_at | BIVM | A.219479_at | KDELC1 | 1.4E−01 | 7.7E−02 | 4.5E−02 | 155 | 94 |
| | | | | | | WVG | | |
| | | | | | | p-value | | |
| | | | | | | 7.52E−10 | 216 | 33 |

Tables 3A1-3C2. Comparison of the SAGC classifier with the currently known classifiers of breast cancer. Parameters in bold font indicate where SAGC classifier outperforms corresponding prototypes or where prototype is unknown. DFS disease free survival; RFS —recurrence free survival; OS —overall survival; DMFS-distant metastasis free survival; DRFS-distant recurrence free survival; parameters (Hazard Ratio (HR), differences in 5 year- and 10 year DFS, Wald, log-rank and likelihood ratio p-values) are highlighted if outperform either in proposal or in prototype.

TABLE 3A1

| | | | | | | 5 years DFS/ | 10 years DFS/ |
|---|---|---|---|---|---|---|---|
| | | | | | | RFS: LR—low | RFS: LR—low |
| | | | Hazard | | | risk group; | risk group; |
| Example | | Training/ | ratio, | Confidence | Wald/log-rank | HR—high risk | HR—high |
| number/classifier | Dataset | Testing | DFS | interval (CI) | p-value | group | risk group |
| 1. Proposed: | ER“+”LN“−” breast cancer | Training | 4.0 | (1.37-11.74) | Wald p = 0.01; | LR: 92% DFS; | LR: 80% DFS; |
| ccSAGP | patients treated with | | | | Log-rank | HR: 63% DFS | HR: 63% DFS |
| RNF139/ | tamoxifen; Joined Uppsala | | | | p = 0.006 | Difference: | Difference: |
| TATDN1 | and Stockholm cohorts, | | | | | 29% | 17% |
| | n = 74 | | | | | | |
| | ER“+”LN“−” breast cancer | Testing | 4.2 | (1.61-10.0) | Wald | LR: 89% DFS | LR: 71% |
| | patients treated with | | | | p = 0.003; | HR: 54% DFS | HR: < |
| | tamoxifen; Oxford cohort, | | | | Log-rank | Difference: 35% | or = 43% |
| | n = 66 | | | | p = 0.001 | | Difference: > |
| | | | | | | | or = 28% |
| Prototype: | ER“+”LN“−” breast cancer | Training | 3.9 | (1.5-10.3) | Log-rank | LR: 91% | LR: 90% |
| HOXB13/IL17BR | patients treated with | | | | P = 0.007 | HR: 81% | HR: 54% |
| | tamoxifen, n = 122 | | | | | Difference: 10% | Difference: |
| | | | | | | | 36% |
| | ER“+”LN“−” breast cancer | Testing | 2.03 | (1.15-3.59) | Log-rank | LR: 94% DFS; | LR: 78% DFS; |
| | patients treated with | | | | p = 0.015 | HR: 64% DFS | HR: 50% DFS |
| | tamoxifen, n = 130 | | | | | Difference: 30% | Difference: |
| | | | | | | | 28% |
| 2. Proposed: | ER“+”LN“−” breast cancer | Training | 27.9 (DFS, | (7.02-110.7) | Wald p = 7.3E−06; | LR: 95% | LR: 84% |
| 12 ccSAGPs | patients treated with | | multivariate | | Likelihood ratio | HR: 27% | HR: < or = |
| (SAGC) | tamoxifen; joined Uppsala | | with | | p = 2.3E−06 | Difference: 68% | 19% |
| | and Stockholm cohorts, | | tumor | | | | Difference: < |
| | n = 74 | | size | | | | or = 65% |
| | | | and | | | | |
| | | | age) | | | | |
| | ER“+”LN“−” breast cancer | Testing | 4.8 | (1.8-12.7) | Wald | LR: 88% DFS; | LR: 68%% |
| | patients treated with | | (DFS, | | p = 0.0009; | HR: 41% DFS | DFS; |
| | tamoxifen; Oxford cohort, | | multivariate | | Log-rank | Difference: | HR: < or = 41% |
| | n = 66 | | with | | p = 0.0003 | 47% | Difference: > |
| | | | tumor | | | | or = 27% |
| | | | size | | | | |
| | | | and | | | | |
| | | | age*) | | | | |
| Prototype: | ER“+”LN“−” breast cancer | Training | 3.21 | (2.23-4.61) | Likelihood | LR: 98% DRFS; | LR: 94% |
| 21 gene | patients treated with | | (DRFS, | | ratio p = 0.001 | HR: 78% DRFS | DRFS; |
| signature | tamoxifen, n = 668 | | multivariate | | | Difference: | HR: 69% DRFS |
| (Oncotype DX) | | | with | | | 20% | Difference: |
| | | | tumor | | | | 25% |
| | | | size | | | | |
| | | | and | | | | |
| | | | age) | | | | |
| 3. Proposed: | breast cancer patients with | Training | 6.4 | (3.31-12.33) | Wald p = 3.3E−8 | LR: 83% DFS; | LR: 66% |
| 12 ccSAGPs | grade 3 (G3) tumors; | | | | Log-rank | HR: 26% DFS | RFS; |
| (SAGC) | Marseille cohort n = 121 | | | | p = 3.2E−10 | Difference: | HR: 18% RFS |
| | | | | | | 57% | Difference: |
| | | | | | | | 48% |
| | breast cancer patients with | Testing | 2.7 | (1.3-5.6) | Wald p = 0.008 | LR: 63% DFS; | LR: 56% |
| | grade 3 (G3) tumors; joined | | | | Log-rank | HR: 33% DFS | DFS; |
| | Uppsala and Stockholm | | | | p = 0.006 | Difference: | HR: 17% DFS |
| | cohorts, n = 116 | | | | | 30% | Difference: |
| | | | | | | | 39% |
| Prototype: | breast cancer patients with | Training | Non- | Non- | Log-rank | LR: 56% DFS; | LR: 51% DFS; |
| Molecular | G3 tumors; n = 86 | | available | available | p = 0.175(DFS) | HR: 35% DFS | HR: 26% DFS |
| cytogenetic | | | | | | Difference: 21% | Difference: |
| classifier | | | | | | | 25% |

TABLE 3A2

| Example | | | Dataset | Training/ | Classifier |
|---|---|---|---|---|---|
| number/classifier | Dataset | Type of dataset | reference | Testing | reference |
| 1. Proposed: | ER“+”LN“−” breast cancer patients treated | Affymetrix U133A&B microarray | [22, 75] | Training | Current |
| ccSAGP | with tamoxifen; Joined Uppsala and | | | | proposal |
| RNF139/TATDN1 | Stockholm cohorts, n = 74 | | | | |
| | ER“+”LN“−” breast cancer patients treated | Affymetrix U133A&B microarray | [81], | Testing | Current |
| | with tamoxifen; Oxford cohort, n = 66 | | GSE6532 | | proposal |
| Prototype: | ER“+”LN“−” breast cancer patients treated | QRT PCR | NA | Training | Ma et al., |
| HOXB13/IL17BR | with tamoxifen, n = 122 | | | | [17] |
| | ER“+”LN“−” breast cancer patients treated | QRT PCR | NA | Testing | Goetz et |
| | with tamoxifen, n = 130 | | | | al., [55] |
| 2. Proposed: | ER“+”LN“−” breast cancer patients treated | Affymetrix U133A&B microarray | [22, 75] | Training | Current |
| 12 ccSAGPs (SAGC) | with tamoxifen; joined Uppsala and | | | | proposal |
| | Stockholm cohorts, n = 74 | | | | |
| | ER“+”LN“−” breast cancer patients treated | Affymetrix U133A&B microarray | [81], | Testing | Current |
| | with tamoxifen; Oxford cohort, n = 66 | | GSE6532 | | proposal |
| Prototype: | ER“+”LN“−” breast cancer patients treated | QRT PCR | NA | Training | Paik et al., |
| 21 gene signature | with tamoxifen, n = 668 | | | | [39] |
| (Oncotype DX) | | | | | |
| 3. Proposed: | breast cancer patients with grade 3 (G3) | Affymetrix U133Plus microarray | [76] | Training | Current |
| 12 ccSAGPs (SAGC) | tumors; Marseille cohort n = 121 | | | | proposal |
| | breast cancer patients with grade 3 (G3) | Affymetrix U133A&B microarray | (1, 2) | Testing | Current |
| | tumors; joined Uppsala and Stockholm | | | | proposal |
| | cohorts, n = 116 | | | | |
| Prototype: | breast cancer patients with G3 tumors; | CGH analysis of cytokeratin 14- | [40] | Training | Jones et al., |
| Molecular | n = 86 | positive and cytokeratin 14- | | | [40] |
| cytogenetic classifier | | negative tumors | | | |

TABLE 3B1

| | | | Dataset | Training/ | Classifier |
|---|---|---|---|---|---|
| Classifier | Dataset | Type of dataset | reference | Testing | reference |
| Proposed: | G3 and G3-like breast cancer | Affymetrix U133A&B microarray | [22, 75] | Training | Current proposal |
| 4. 12 ccSAGPs | patients, Stockholm cohort, n = 58 | | | | |
| (SAGC) | G3 and G3-like breast cancer | Affymetrix U133A&B microarray | [22, 75] | Testing | Current proposal |
| | patients, Uppsala cohort, n = 97 | | | | |
| Prototype: | | | | | |
| unknown | | | | | |
| Proposed: | G1 and G1-like breast cancer | Affymetrix U133A&B microarray | [22, 75] | Training | Current proposal |
| 5. 12 ccSAGPs | patients, Stockholm cohort, n = 88 | | | | |
| (SAGC) | G1 and G1-like breast cancer | Affymetrix U133A&B microarray | [22, 75] | Testing | Current proposal |
| | patients, Uppsala cohort, n = 152 | | | | |
| Prototype: | | | | | |
| unknown | | | | | |
| Proposed: | G1 breast cancer patients, joined | Affymetrix U133A&B microarray | [22, 75] | Training | Current proposal |
| 6. 12 ccSAGPs | Uppsala&Stockholm cohorts, n = 96 | | | | |
| (SAGC) | | | | | |
| Prototype: | | | | | |
| unknown | | | | | |
| 7. Proposed: | ER“−” breast cancer patients; | Affymetrix U133Plus microarray | [76] | Training | Current proposal |
| 12 ccSAGPs | Marseille cohort, n = 109 | | | | |
| (SAGC) | ER“−” breast cancer patients; joined | Affymetrix U133A&B microarray | [22, 75] | Testing | Current proposal |
| | Uppsala and Stockholm cohorts, n = 63 | | | | |
| | ER“−” breast cancer patients; | Affymetrix U133A&B microarray | [22] | Testing | Current proposal |
| | Singapore cohort, n = 32 | | | | |
| | ER“−” breast cancer patients; | Affymetrix U133A&B microarray | [22, 75] | Testing | Current proposal |
| | combined | | | combined | |
| | (Uppsala&Stockholm and Singapore), | | | (Uppsala&Stockholm | |
| | n = 95 | | | and | |
| | | | | Singapore | |
| | | | | datasets) | |
| Prototype: | ER“−” breast cancer patients, n = 186 | Affymetrix U133A | [41] | Training(joined | Teschendorff et |
| Seven-gene | | Agilent custom microarray | | 3 datasets) | al. [41] |
| immune | ER“−” breast cancer patients, | Affymetrix U133A&B, | [41] | Testing | Teschendorff et |
| response | (combined 6 datasets), n = 469 | Affymetrix U133A microarrays | | (combined 6 | al. [41] |
| module | | | | datasets) | |
| 8. Proposed: | breast cancer patients with basal-like | Affymetrix U133Plus microarray | [76] | Training | Current proposal |
| 12 ccSAGPs | grade 3 breast tumors; Marseille | | | | |
| (SAGC) | cohort n = 64 | | | | |
| | breast cancer patients with basal-like | Affymetrix U133A&B microarray | [22, 75] | Testing | Current proposal |
| | grade 3 breast tumors; joined | | | | |
| | Uppsala and Stockholm cohorts, n = 43 | | | | |
| Prototype 1: | breast cancer patients with basal-like | Affymetrix U133A | [42], | Testing | Hallett et al. [42] |
| 14-gene | breast tumors obtained from joined | microarray | GSE1456, | | |
| signature | dataset, n = 49 | | GSE2034, | | |
| | | | GSE3494, | | |
| | | | GSE6532 | | |

TABLE 3B2

| | | | | | | | 10 years |
|---|---|---|---|---|---|---|---|
| | | | Hazard | | Wald/log- | 5 years | DFS/RFS: |
| | | | ratio | | rank/ | DFS/RFS: | LR—low |
| | | | (survival | | likelihood | LR—low risk | risk group; |
| | | | end- | Confidence | ratio | group; HR—high | HR—high |
| Classifier | Dataset | Training/Testing | point) | interval (CI) | p-value | risk group | risk group |
| Proposed: | G3 and G3-like breast | Training | 14.5 | (5.76-36.53) | Wald | LR: 80% DFS; | NA |
| 4.12 | cancer patients, Stockholm | | | | p = 1.4E−8 | HR: 0% DFS | |
| ccSAGPs | cohort, n = 58 | | | | Log-rank | Difference: | |
| (SAGC) | | | | | p = 7.0-13 | 80% | |
| | G3 and G3-like breast | Testing | 3.0 | (1.69-5.43) | Wald | LR: 60% DFS; | LR: 55% |
| | cancer patients, Uppsala | | | | p = 0.0002 | HR: 21% DFS | DFS; |
| | cohort, n = 97 | | | | Log-rank | Difference: | HR: 5% DFS |
| | | | | | p = 9.7E−05 | 39% | Difference: |
| | | | | | | | 50% |
| Prototype: | | | | | | | |
| unknown | | | | | | | |
| Proposed: | G1 and G1-like breast | Training | 16.1 | (5.64-48.56) | Wald | LR: 95% DFS; | NA |
| 5.12 | cancer patients, Stockholm | | | | p = 3.2E−7 | HR: 30% DFS | |
| ccSAGPs | cohort, n = 88 | | | | Log-rank | Difference: | |
| (SAGC) | | | | | p = 7.1E−12 | 65% | |
| | G1 and G1-like breast | Testing | 3.3 | (1.73-6.16) | Wald | LR: 90% DFS; | LR: 81% |
| | cancer patients, Uppsala | | | | p = 0.00025 | HR: 71% DFS | DFS; |
| | cohort, n = 152 | | | | Log-rank | Difference: | HR: 56% DFS |
| | | | | | p = 0.0001 | 19% | Difference: |
| IP1 | | | | | | | 25% |
| Prototype: | | | | | | | |
| unknown | | | | | | | |
| Proposed: | G1 breast cancer patients, | Training | 17.4 | (5.67-53.62) | Wald | LR: 99% DFS; | LR: 94% |
| 6.12 | joined Uppsala&Stockholm | | | | p = 6.1E−7 | HR: 64% DFS | DFS; |
| ccSAGPs | cohorts, n = 96 | | | | Log-rank | Difference: | HR: 28% DFS |
| (SAGC) | | | | | p = 1.6E−11 | 35% | Difference: |
| | | | | | | | 66% |
| Prototype: | ER“−” breast cancer | Training | 0.12 | (0.06-0.22) | Wald | LR: 73% DFS; | LR: 67% |
| unknown | patients; Marseille cohort, | | | | p = 1.2E−10 | HR: 5% DFS | DFS; |
| 7. Proposed: | n = 109 | | | | Log-rank | Difference: | HR: 5% DFS |
| 12 ccSAGPs | | | | | p = 3.0E−14 | 68% | Difference: |
| (SAGC) | | | | | | | 62% |
| | ER“−” breast cancer | Testing | 0.24 | (0.10-0.58) | Wald | LR: 76% DFS; | LR: 69% |
| | patients; joined Uppsala | | | | p = 0.001 | HR: 34% DFS | DFS; |
| | and Stockholm cohorts, | | | | Log-rank | Difference: | HR: < or = 25% |
| | n = 63 | | | | p = 0.0006 | 42% | DFS |
| | | | | | | | Difference: > |
| | | | | | | | or = 44% DFS |
| | ER“−” breast cancer | Testing | 0.17 | (0.05-0.65) | Wald | LR: 84% DFS; | LR: NA; |
| | patients; Singapore cohort, | | | | p = 0.009 | HR: 33% DFS | HR: NA |
| | n = 32 | | | | Log-rank | Difference: | Difference: NA |
| | | | | | p = 0.003 | 41% | |
| | ER“−” breast cancer | Testing combined | 0.22 | (0.11-0.45) | Wald | LR: 79% DFS; | LR: 72% |
| | patients; combined | (Uppsala&Stockholm | | | p = 2.9E−05 | HR: 34% DFS | DFS; |
| | (Uppsala&Stockholm and | and Singapore | | | Log-rank | Difference: | HR: < or = 26% |
| | Singapore), n = 95 | datasets) | | | p = 6.03E−6 | 45% | DFS |
| | | | | | | | Difference: |
| | | | | | | | > or = 56% |
| Prototype: | ER“−” breast cancer | Training(joined 3 | 0.30 | (0.16-0.56) | Log-rank | Good-up: 83% | Good- |
| Seven-gene | patients, n = 186 | datasets) | | | p = 0.0002 | DFS; | up: 80% DFS; |
| immune | | | | | | poor: 51% DFS | poor: 34% DFS |
| response | | | | | | Difference: | Difference: |
| module | | | | | | 32% | 46% |
| | ER“−” breast cancer | Testing (combined 6 | 0.15 | (0.07-0.36) | Log-rank | Good- | Good- |
| | patients, (combined 6 | datasets) | | | p = 1.0E−06 | up: 99%% | up: 94% |
| | datasets), n = 469 | | | | | DFS; | DFS; |
| | | | | | | poor: 63% DFS | poor: 50% DFS |
| | | | | | | Difference: | Difference: |
| | | | | | | 36% | 44% |
| 8. Proposed: | breast cancer patients with | Training | 15.6 or | (5.67-42.77) | Wald | LR: 81% DFS; | LR: 81% |
| 12 ccSAGPs | basal-like grade 3 breast | | 0.06 | or (0.02-0.18) | p = 1.0E−07 | HR: 9% DFS | DFS; |
| (SAGC) | tumors; Marseille cohort | | | | Log-rank | Difference: | HR: 0% DFS |
| | n = 64 | | | | p = 5.4E−12 | 72% | Difference: |
| | | | | | | | 0% |
| | breast cancer patients with | Testing | 5.5 or | (1.89-15.92) | Wald p = | LR: 81% DFS; | LR: 75% |
| | basal-like grade 3 breast | | 0.18 | or | 0.0018 | HR: 34% DFS | DFS; |
| | tumors; joined Uppsala and | | | (0.06-0.53) | Log-rank | Difference: | HR: 23% DFS |
| | Stockholm cohorts, n = 43 | | | | p = 4.8E−04 | 47% | Difference: |
| | | | | | | | 52 |
| Prototype 1: | breast cancer patients with | Testing | 4.7 | (1.8-12.3) | Log-rank | LR: ≈94% DFS; | NA |
| 14-gene | basal-like breast tumors | | | | p = 0.0017 | HR: 50% DRFS | |
| signature | obtained from joined | | | | | Difference: | |
| | dataset, n = 49 | | | | | 44% | |

TABLE 3C1

| | | | Dataset | Training/ | Classifier |
|---|---|---|---|---|---|
| Classifier | Dataset | Type of dataset | reference | Testing | reference |
| Prototype 2: | breast cancer patients with basal-like | Affymetrix U133Plus | [76] | Training | Sabatier et al. |
| 28-kinase immune | breast tumors; Marseille cohort n = 73 | microarray | | | [15] |
| metagene | breast cancer patients with basal-like | Affymetrix U133A, | [15] | Testing | Sabatier et al. |
| | breast tumors; joined set from | Affymetrix U133A&B | | | [15] |
| | different sources, n = 380 | microarrays | | | |
| 9. Proposed: | breast cancer patients with Luminal A | Affymetrix U133Plus | [76] | Training | Current proposal |
| 12 ccSAGPs (SAGC) | breast tumors; Marseille cohort n = 84 | microarray | | | |
| | breast cancer patients with Luminal A | Affymetrix U133A&B | (1, 2) | Testing | Current proposal |
| | breast tumors; joined Uppsala and | microarray | | | |
| | Stockholm cohorts, n = 121 | | | | |
| Prototype: | breast cancer patients with Luminal A | Affymetrix U133Plus | [14] | Training | Finetti et al. [14] |
| Sixteen kinase gene | breast tumors; Marseille cohort n = 80 | microarray | | | |
| expression classifier | breast cancer patients with Luminal A | Custom Agilent microarray, | [14] | Testing | Finetti et al. [14] |
| | breast tumors; 3 joined datasets, | Affymetrix U133A, | | | |
| | n = 276 | Affymetrix U133A&B | | | |
| | | microarrays | | | |
| 10. Proposed: | ER“+”LN“−”PgR“+”, breast cancer | Affymetrix U133A&B | (1, 2) | Training | Current proposal |
| 12 ccSAGPs (SAGC) | patients with tumors <=2 cm, who did | microarray | | | |
| | not receive any systemic treatment, | | | | |
| | joined Uppsala and Stockholm cohort, | | | | |
| | n = 81 | | | | |
| Prototype: | | | | | |
| unknown | | | | | |
| 11. Proposed: | Colon cancer stage II tumors, | Affymetrix U133Plus | [43] | Training | Current proposal |
| 12 ccSAGPs(SAGC) | Amsterdam cohort, | microarray | | | |
| | N = 89 | | | | |
| Prototype: Colon | Colon cancer stage II tumors, | Affymetrix U133Plus | [43] | Training | [43] |
| cancer stem cell gene | Amsterdam cohort, | microarray | | | |
| signature (187 genes) | N = 89 | | | | |
| 12. Proposed: | Non-small Cell Lung tumors, | Affymetrix U133Plus | [44] | Training | Current proposal |
| 12 ccSAGPs(SAGC) | Rotterdam cohort, | microarray | | | |
| | N = 82 | | | | |
| Prototype: | Non-small Cell Lung tumors, | Affymetrix U133Plus | [44] | Training | [44] |
| Non-small lung cancer | Rotterdam cohort, | microarray | | | |
| 17-gene signature | N = 82 | | | | |

TABLE 3C2

| | | | | | | 5 years | |
|---|---|---|---|---|---|---|---|
| | | | | | | DFS*/RFS: | |
| | | | | | Wald/log- | LR—low risk | 10 years |
| | | | Hazard ratio | | rank/ | group; | DFS/RFS: |
| | | Training/ | (survival | Confidence | likelihood | HR—high risk | LR—low risk group; |
| Classifier | Dataset | Testing | end-point) | interval (CI) | ratio p-value | group | HR—high risk group |
| Prototype2: | breast cancer patients with | Training | 0.21 | (0.06-0.70) | Log-rank | LR: 91% DFS; | 8 year DFS: |
| 28-kinase | basal-like breast tumors; | | | | p = 0.005 | HR: 49% DFS | LR: 91% DFS; |
| immune | Marseille cohort n = 73 | | | | | Difference: | HR: 49% DFS |
| metagene | | | | | | 42% | Difference: 42% |
| | breast cancer patients with | Testing | 0.45 | (0.29-0.69) | Log-rank | LR: 78% DFS; | 8 year DFS: |
| | basal-like breast tumors; | | | | p = 1.6E−04 | HR: 54% DFS | LR: 78% RFS; |
| | joined set from different | | | | | Difference: | HR: 50% RFS |
| | sources, n = 380 | | | | | 24% | Difference: 28% |
| 9. Proposed: | breast cancer patients with | Training | 13.0 | (5.35-31.54) | Wald | LR: 87% DFS; | LR: 78% DFS; |
| 12 ccSAGPs | Luminal A breast tumors; | | | | p = 1.5E−08 | HR: 22% DFS | HR: 0% DFS |
| (SAGC) | Marseille cohort n = 84 | | | | Log-rank | Difference: | Difference: 78% |
| | | | | | p = 2.4E−12 | 65% | |
| | breast cancer patients with | Testing | 3.36 | (1.59-7.12) | Wald | LR: 89% DFS; | LR: 73% DFS; |
| | Luminal A breast tumors; | | | | p = 0.0016 | HR: 52% DFS | HR: 41% DFS |
| | joined Uppsala and | | | | Log-rank | Difference: | Difference: 22% |
| | Stockholm cohorts, n = 121 | | | | p = 7.9E−4 | 37% | |
| Prototype: | breast cancer patients with | Training | 7.77 | (1.97-30.66) | Log-rank | LR: 83% DFS; | LR: 79% DFS; |
| Sixteen kinase | Luminal A breast tumors; | | | | p = 0.003 | HR: 65% DFS | HR: 54% DFS |
| gene | Marseille cohort n = 80 | | | | | Difference: | Difference: 25% |
| expression | | | | | | 18% | |
| classifier | breast cancer patients with | Testing | 4.84 | (2.13-11.0) | Log-rank | LR: 87% RFS; | LR: 78% RFS; |
| | Luminal A breast tumors; 3 | | | | p = 1.7E−05 | HR: 69% RFS | HR: 44% RFS |
| | joined datasets, n = 276 | | | | | Difference: | Difference: 34% |
| | | | | | | 18% | |
| 10. Proposed: | ER“+”LN“−”PgR“+”, breast | Training | 16.3 | (6.20-42.9) | Wald | LR: 88% DFS; | LR: 79% DFS; |
| 12 ccSAGPs | cancer patients with tumors | | | | p = 1.6E−08 | HR: 10% DFS | HR: 0% DFS |
| (SAGC) | <=2 cm, who did not receive | | | | Log-rank | Difference: | Difference: 79% |
| | any systemic treatment, | | | | p = 7.5E−14 | 78% | |
| | joined Uppsala and | | | | | | |
| | Stockholm cohort, n = 81 | | | | | | |
| Prototype: | | | | | | | |
| unknown | | | | | | | |
| 11. Proposed: | Colon cancer stage II tumors, | Training | 13.6 | (5.2-35.3) | Wald | LR: 84% RFS; | LR: 84% RFS; |
| 12 ccSAGPs | Amsterdam cohort, | | | | p = 9.4E−08 | HR: < or = 11% | HR: < or = 11% RFS |
| (SAGC) | N = 89 | | | | Log rank | RFS | Difference: > or = |
| | | | | | p = 7.9E−12 | Difference: > or = | 73% RFS |
| | | | | | | 73% RFS | |
| Prototype: Colon | Colon cancer stage II tumors, | Training | NA | NA | Log rank | LR: 87% RFS; | NA |
| cancer stem cell | Amsterdam cohort, | | | | p = 6.7E−03 | HR: <=57% RFS | |
| gene signature | N = 89 | | | | | Difference: | |
| (187 genes) | | | | | | <=30% RFS | |
| 12. Proposed: | Non-small Cell Lung tumors, | Training | 6.4 | (3.42-12.04) | Wald | LR: 63% OS; | LR: 40% OS; |
| 12 ccSAGPs | Rotterdam cohort, | | | | p = 6.8E−9 | HR: 5% OS | HR: 0% OS |
| (SAGC) | N = 82 | | | | | Difference: 58% | Difference: 40% |
| | | | | | | OS | OS |
| Prototype: | Non-small Cell Lung tumors, | Training | NA | NA | Wald | LR: 67% OS; | LR: 45% OS; |
| Non-small lung | Rotterdam cohort, | | | | p = 5.6E−06 | HR: <=21% OS | HR: <=8% OS |
| cancer 17-gene | N = 82 | | | | | Difference: | Difference: <=37% OS |
| signature | | | | | | <=46% OS | |

TABLE 4
Publicly available microarray datasets referred to herein

| | | | series | |
|---|---|---|---|---|
| | | sample | accession | |
| Description of dataset | Type of microarray | size, n | ID | Ref. |
| Breast cancer patients | Gene expression | 249 | GSE4922 | [22, 75] |
| (Uppsala cohort) | microarray, Affymetrix | | | |
| | U133A&B | | | |
| Breast cancer patients | Gene expression | 159 | GSE1456 | [22, 75] |
| (Stockholm cohort) | microarray, Affymetrix | | | |
| | U133A&B | | | |
| Breast cancer patients | Gene expression | 47 | GSE3744 | [77] |
| (Harvard cohort 1) | microarray, Affymetrix | | | |
| | U133 Plus 2.0 | | | |
| Breast cancer patients | Gene expression | 115 | GSE19615 | [78] |
| (Harvard cohort 2) | microarray, Affymetrix | | | |
| | U133 Plus 2.0 | | | |
| Breast cancer patients | Gene expression | 266 | GSE21653 | [76] |
| (Marseille cohort) | microarray, Affymetrix | | | |
| | U133 Plus 2.0 | | | |
| Breast cancer patients | Gene expression | 88 | GSE4922 | [22] |
| (Singapore cohort) | microarray, Affymetrix | | | |
| | U133A&B | | | |
| Breast cancer patients | Gene expression | 178 | GSE6532 | [82] |
| (Oxford cohort within the | microarray, Affymetrix | | | |
| large joined dataset) | U133A&B | | | |
| Colon cancer patients | Gene expression | 89 | GSE33114 | [43] |
| (Amsterdam Cohort) | microarray, Affymetrix | | | |
| | U133 Plus 2.0 | | | |
| Non-small lung cancer | Gene expression | 82 | GSE19188 | [44] |
| patients (Rotterdam cohort) | microarray, Affymetrix | | | |
| | U133 Plus 2.0 | | | |
| OriGene cohort | Gene expression | 62 | GSE61304 | Current |
| | microarray, Affymetrix | | | report |
| | U133 Plus 2.0 | | | |

TABLE 5
List of robust survival significant SAGPs from SAGC in each specific subpopulation of breast tumors. They represent the
“core” SAGPs for each subpopulation.

| | | | | cut- | cut- | | | 2D_P- | Cut- | cut- | | | 2D_P- |
|---|---|---|---|---|---|---|---|---|---|---|---|---|---|
| AFFyID1 | AFFyID2 | Gene_Name1 | Gene_Name2 | off1 | off2 | beta1 | design | value | off1 | off2 | beta1 | design | value |
| | | | | Training group (joined | | | | | | | | | |
| ER+ LN− tamoxifen treated breast tumors | | | | Uppsala&Stockholm) | | | | | Testing group (Oxford cohort) | | | | |
| A.209510_at | B.223231_at | RNF139 | TATDN1 | 8.1 | 8.14 | −0.05 | 6.2 | 0.01 | 8.1 | 8.18 | −0.05 | 6.2 | 0.003 |
| Grade 3 breast tumors | | | | Training group (Marseille cohort) | | | | | Testing group (joined | | | | |
| B.226481_at | A.202689_at | VPRBP | RBM15B | 8.58 | 8.39 | 0.16 | 3.1 | 0.0015 | 8.6 | 8.19 | 0.16 | 3.1 | 0.015 |
| G3G3like breast tumors | | | | Training group (Stockholm cohort) | | | | | Testing group (Uppsala cohort) | | | | |
| B.232348_at | A.202679_at | C18orf8 | NPC1 | 3.98 | 7.62 | −0.27 | 3.1 | 0.002 | 3.96 | 7.59 | −0.27 | 3.1 | 0.04 |
| B.234464_s_at | B.234812_at | EME1 | LRRC59 | 7.95 | 3.88 | 0.27 | 7.1 | 0.024 | 8 | 3.86 | 0.27 | 7.1 | 0.01 |
| G1G1 like | | | | Training group (Stockholm cohort) | | | | | Testing group (Uppsala cohort) | | | | |
| A.217304_at | B.227304_at | SHMT1 | SMCR8 | 5.35 | 6.79 | 0.38 | 4.1 | 0.004 | 5.34 | 6.77 | 0.38 | 4.1 | 0.036 |
| ER− breast tumors | | | | Training group (Marseille cohort) | | | | | Testing group (joined | | | | |
| A.204925_at | A.209154_at | CTNS | TAX1BP3 | 6.08 | 9.2 | 0.65 | 6.2 | 0.001 | 6.19 | 9.17 | 0.65 | 6.2 | 0.017 |
| Basal-like G3 breast tumors | | | | Training group (Marseille cohort) | | | | | Testing group (Singapore cohort) | | | | |
| A.204925_at | A.209154_at | CTNS | TAX1BP3 | 6.4 | 9.52 | −0.16 | 6.2 | 0.010 | 6.4 | 9.4 | −0.16 | 6.2 | 0.018 |
| A.209510_at | B.223231_at | RNF139 | TATDN1 | 7.74 | 8.49 | −0.51 | 5.1 | 0.015 | 7.74 | 8.47 | −0.51 | 5.1 | 0.022 |
| Luminal A breast tumors | | | | Training group (Marseille cohort) | | | | | Testing group (joined | | | | |
| B.222761_at | A.219479_at | BIVM | KDELC1 | 8.67 | 5.28 | −0.05 | 6.1 | 0.016 | 8.66 | 5.23 | −0.05 | 6.1 | 0.018 |

TABLE 6
Functional annotation analysis using the DAVID bioinformatics software of 978 differentially
expressed, significantly upregulated genes in high-risk-vs. low-risk-subgroups obtained from
3 total BC cohorts (Uppsala, Stockholm and Metadata).

| | | Genes | | |
|---|---|---|---|---|
| | | annotated | | |
| | | by | Bonferroni | Fold |
| Category | Term | DAVID | corr._pValue | Enrichment |
| KEGG_PATHWAY | hsa03050: Proteasome | 27 | 5.53E−17 | 8.57 |
| | hsa04110: Cell cycle | 39 | 3.31E−14 | 4.65 |
| | hsa03030: DNA replication | 19 | 2.06E−10 | 7.87 |
| | hsa03040: Spliceosome | 25 | 8.47E−05 | 3.08 |
| | hsa03430: Mismatch repair | 11 | 1.22E−04 | 7.13 |
| | hsa00240: Pyrimidine metabolism | 20 | 1.77E−03 | 3.14 |
| | hsa00970: Aminoacyl-tRNA | 12 | 7.71E−03 | 4.36 |
| | biosynthesis | | | |
| | hsa00230: Purine metabolism | 25 | 9.43E−03 | 2.44 |
| | hsa04114: Oocyte meiosis | 20 | 1.49E−02 | 2.71 |
| GOTERM_BP_FAT | GO: 0000278~mitotic cell cycle | 128 | 2.11E−61 | 5.87 |
| | GO: 0007049~cell cycle | 176 | 2.01E−55 | 3.85 |
| | GO: 0000280~nuclear division | 85 | 1.73E−43 | 6.56 |
| | GO: 0006260~DNA replication | 52 | 2.22E−17 | 4.65 |
| | GO: 0043161~proteasomal ubiquitin- | 34 | 2.79E−13 | 5.66 |
| | dependent protein catabolic process | | | |
| | GO: 0006974~response to DNA damage | 64 | 4.83E−11 | 2.91 |
| | stimulus | | | |
| | GO: 0006281~DNA repair | 50 | 1.93E−08 | 2.99 |
| | GO: 0008380~RNA splicing | 49 | 6.78E−08 | 2.93 |
| | GO: 0042254~ribosome biogenesis | 25 | 3.63E−04 | 3.48 |
| | GO: 0030163~protein catabolic process | 70 | 5.59E−04 | 1.91 |
| | GO: 0006096~glycolysis | 13 | 3.04E−02 | 4.69 |
| GOTERM_CC_FAT | GO: 0005739~mitochondrion | 152 | 1.66E−23 | 2.44 |
| | GO: 0044429~mitochondrial part | 97 | 1.83E−18 | 2.84 |
| | GO: 0000502~proteasome complex | 30 | 9.84E−18 | 8.56 |
| | GO: 0000779~condensed chromosome, | 30 | 1.52E−16 | 7.92 |
| | centromeric region | | | |
| | GO: 0000776~kinetochore | 32 | 2.00E−16 | 7.24 |
| | GO: 0015630~microtubule cytoskeleton | 80 | 7.93E−12 | 2.54 |
| | GO: 0005759~mitochondrial matrix | 45 | 3.01E−10 | 3.45 |
| | GO: 0005681~spliceosome | 24 | 7.39E−04 | 3.17 |
| GOTERM_MF_FAT | GO: 0000166~nucleotide binding | 213 | 3.43E−15 | 1.74 |
| | GO: 0003723~RNA binding | 94 | 1.68E−12 | 2.40 |
| | GO: 0004549~tRNA-specific | 8 | 7.56E−04 | 12.24 |
| | ribonuclease activity | | | |
| INTERPRO | IPR001353: Proteasome, subunit | 13 | 2.54E−08 | 12.79 |
| | alpha/beta | | | |
| | IPR017998: Chaperone, tailless complex | 8 | 4.69E−04 | 13.60 |
| | polypeptide 1 | | | |
| | IPR002194: Chaperonin TCP-1, | 7 | 5.63E−03 | 13.09 |
| | conserved site | | | |
| | IPR016050: Proteasome, beta-type | 7 | 2.24E−02 | 10.91 |
| | subunit, conserved site | | | |
| | IPR018525: DNA-dependent ATPase | 6 | 2.94E−02 | 14.02 |
| | MCM, conserved site | | | |
| SP_PIR_KEYWORDS | acetylation | 393 | 4.09E−99 | 2.98 |
| | cell cycle | 116 | 2.26E−46 | 5.03 |
| | phosphoprotein | 572 | 1.28E−41 | 1.57 |
| | cell division | 84 | 3.81E−41 | 6.36 |
| | nucleus | 388 | 1.73E−35 | 1.81 |
| | mitochondrion | 132 | 3.34E−30 | 3.17 |
| | proteasome | 30 | 1.13E−20 | 10.70 |
| | dna replication | 30 | 5.20E−14 | 6.81 |
| | Chaperone | 39 | 1.04E−13 | 4.93 |
| | ubl conjugation | 73 | 7.89E−10 | 2.48 |
| | mrna splicing | 36 | 1.33E−07 | 3.44 |
| | mitochondrion inner membrane | 34 | 2.47E−07 | 3.52 |
| | DNA damage | 34 | 1.22E−06 | 3.31 |
| UP_SEQ_FEATURE | transit peptide: Mitochondrion | 82 | 8.72E−20 | 3.49 |
| | mutagenesis site | 188 | 2.38E−13 | 1.83 |

Tables 7. The optimal classification parameters for SAGC (partition design, rotation angle, and gene expression cut-offs) for 2-D RDDg procedure¹. Selected twelve pairs of Affyprobesets have been used for subsequent Weighted Voting Grouping in each group. Comments: ¹—for description of the method see Materials and Methods section; ²-optimal cut-off for expression value for the corresponding Affyprobeset. ³-rotation angle coefficient in the 2 RDDg procedure. ⁴-one of 7 possible two-group designs (see materials and methods section). ⁵-gene expression data were not Log 2-transformed; gene pairs in which expression values were <=50 were excluded from the consequent WVG procedure. ⁶-expression data for each probeset were displayed as the log-2 of the deviations to the calculated geometric means for that of probesets.

TABLE 7A
The optimal SAGC classification parameters for ER“+”, LN“−” breast cancer
patients who received adjuvant systemic tamoxifen treatment after curative surgery.

| | | | | | | | | | 2-D |
|---|---|---|---|---|---|---|---|---|---|
| | | | | | | | | | RDDg |
| | Affymetrix | Affymetrix | | | | | | | Wald |
| | probeset for | probeset for | Gene | Gene | cut- | cut- | | | p- |
| pair | gene 1 | gene 2 | symbol 1 | symbol 2 | off1² | off2² | beta1³ | design⁴ | value |
| 1 | B.232348_at | A.202679_at | C18orf8 | NPC1 | 4.1 | 7.5 | 0.27 | 2.2 | 9.2E−03 |
| 2 | A.219544_at | A.218362_s_at | BORA | DIS3 | 6.1 | 5.1 | −0.27 | 3.1 | 3.3E−04 |
| 3 | A.209971_x_at | A.217736_s_at | AIMP2 | EIF2AK1 | 8.3 | 9.2 | −0.16 | 1.1 | 8.2E−04 |
| 4 | A.217304_at | B.227304_at | SHMT1 | SMCR8 | 5.0 | 6.7 | −0.38 | 6.1 | 3.6E−03 |
| 5 | A.209690_s_at | A.208996_s_at | DOK4 | POLR2C | 4.6 | 7.9 | 0.00 | 5.1 | 3.1E−03 |
| 6 | B.228019_s_at | B.226521_s_at | MRPS18C | FAM175A | 8.2 | 6.9 | 0.00 | 6.1 | 1.2E−02 |
| 7 | A.204925_at | A.209154_at | CTNS | TAX1BP3 | 6.7 | 8.8 | 0.81 | 1.1 | 1.2E−03 |
| 8 | B.234464_s_at | B.234812_at | EME1 | LRRC59 | 7.4 | 4.6 | 0.00 | 2.2 | 3.2E−03 |
| 9 | B.226481_at | A.202689_at | VPRBP | RBM15B | 8.4 | 7.8 | −0.38 | 2.2 | 6.1E−03 |
| 10 | A.209510_at | B.223231_at | RNF139 | TATDN1 | 8.1 | 8.1 | −0.05 | 6.2 | 1.1E−02 |
| 11 | A.201139_s_at | A.221570_s_at | SSB | METTL5 | 7.6 | 8.2 | 0.00 | 1.1 | 1.5E−02 |
| 12 | B.222761_at | A.219479_at | BIVM | KDELC1 | 7.9 | 6.1 | 0.00 | 3.1 | 5.4E−03 |

TABLE 7B
The optimal SAGC classification parameters for breast cancer patients
histological Grade 3 breast tumors.

| | | | | | | | | | 2-D |
|---|---|---|---|---|---|---|---|---|---|
| | | | | | | | | | RDDg |
| | Affymetrix | Affymetrix | | | | | | | Wald |
| | probeset for | probeset for | Gene | Gene | cut- | cut- | | | p- |
| pair | gene 1 | gene 2 | symbol 1 | symbol 2 | off1 | off2 | beta1 | design | value |
| 1 | B.232348_at | A.202679_at | C18orf8 | NPC1 | 3.6 | 7.5 | 0.00 | 7.1 | 1.7E−02 |
| 2 | A.219544_at | A.218362_s_at | BORA | DIS3 | 5.9 | 6.5 | 0.00 | 6.2 | 1.8E−02 |
| 3 | A.209971_x_at | A.217736_s_at | AIMP2 | EIF2AK1 | 8.8 | 9.2 | −0.16 | 7.2 | 3.1E−03 |
| 4 | A.217304_at | B.227304_at | SHMT1 | SMCR8 | 5.8 | 6.3 | 0.38 | 7.2 | 1.7E−02 |
| 5 | A.209690_s_at | A.208996_s_at | DOK4 | POLR2C | 5.1 | 8.1 | −0.38 | 5.1 | 2.6E−03 |
| 6 | B.228019_s_at | B.226521_s_at | MRPS18C | FAM175A | 8.0 | 6.8 | 0.00 | 4.1 | 1.1E−03 |
| 7 | A.204925_at | A.209154_at | CTNS | TAX1BP3 | 6.9 | 9.6 | 0.00 | 5.1 | 8.3E−03 |
| 8 | B.234464_s_at | B.234812_at | EME1 | LRRC59 | 8.2 | 4.7 | 0.00 | 2.2 | 4.0E−03 |
| 9 | B.226481_at | A.202689_at | VPRBP | RBM15B | 8.6 | 8.4 | 0.16 | 3.1 | 1.5E−03 |
| 10 | A.209510_at | B.223231_at | RNF139 | TATDN1 | 8.0 | 8.8 | 0.00 | 3.2 | 6.9E−03 |
| 11 | A.201139_s_at | A.221570_s_at | SSB | METTL5 | 6.8 | 7.0 | 0.00 | 4.1 | 5.3E−03 |
| 12 | B.222761_at | A.219479_at | BIVM | KDELC1 | 8.8 | 5.6 | 0.00 | 4.2 | 1.6E−02 |

TABLE 7C
The optimal SAGC classification parameters for breast cancer patients
with Grade 3 and Grade 3-like breast tumors.

| | | | | | | | | | 2-D |
|---|---|---|---|---|---|---|---|---|---|
| | | | | | | | | | RDDg |
| | Affymetrix | Affymetrix | | | | | | | Wald |
| | probeset for | probeset for | Gene | Gene | cut- | cut- | | | p- |
| pair | gene 1 | gene 2 | symbol 1 | symbol 2 | off1 | off2 | beta1 | design | value |
| 1 | B.232348_at | A.202679_at | C18orf8 | NPC1 | 4.0 | 7.6 | −0.27 | 3.1 | 2.4E−03 |
| 2 | A.219544_at | A.218362_s_at | BORA | DIS3 | 6.5 | 5.0 | −0.38 | 3.1 | 4.7E−03 |
| 3 | A.209971_x_at | A.217736_s_at | AIMP2 | EIF2AK1 | 8.2 | 8.8 | 0.00 | 7.2 | 1.2E−02 |
| 4 | A.217304_at | B.227304_at | SHMT1 | SMCR8 | 5.2 | 7.0 | 0.00 | 3.2 | 2.8E−02 |
| 5 | A.209690_s_at | A.208996_s_at | DOK4 | POLR2C | 4.3 | 8.0 | 0.00 | 6.2 | 7.0E−04 |
| 6 | B.228019_s_at | B.226521_s_at | MRPS18C | FAM175A | 8.3 | 6.7 | 0.00 | 7.2 | 7.8E−03 |
| 7 | A.204925_at | A.209154_at | CTNS | TAX1BP3 | 6.7 | 9.3 | −0.05 | 6.2 | 1.2E−02 |
| 8 | B.234464_s_at | B.234812_at | EME1 | LRRC59 | 8.0 | 3.9 | 0.27 | 7.1 | 2.4E−02 |
| 9 | B.226481_at | A.202689_at | VPRBP | RBM15B | 8.3 | 7.6 | −0.65 | 2.2 | 1.3E−02 |
| 10 | A.209510_at | B.223231_at | RNF139 | TATDN1 | 7.8 | 7.8 | 0.81 | 2.2 | 1.7E−02 |
| 11 | A.201139_s_at | A.221570_s_at | SSB | METTL5 | 7.3 | 8.1 | 0.00 | 7.2 | 4.5E−02 |
| 12 | B.222761_at | A.219479_at | BIVM | KDELC1 | 7.8 | 6.2 | 0.00 | 1.1 | 2.1E−02 |

TABLE 7D
The optimal SAGC classification parameters for breast cancer patients
with Grade 1 and Grade 1-like breast tumors.

| | | | | | | | | | 2-D |
|---|---|---|---|---|---|---|---|---|---|
| | | | | | | | | | RDDg |
| | Affymetrix | Affymetrix | | | | | | | Wald |
| | probeset for | probeset for | Gene | Gene | cut- | cut- | | | p- |
| pair | gene 1 | gene 2 | symbol 1 | symbol 2 | off1 | off2 | beta1 | design | value |
| 1 | B.232348_at | A.202679_at | C18orf8 | NPC1 | 3.7 | 7.8 | 0.00 | 6.2 | 4.5E−02 |
| 2 | A.219544_at | A.218362_s_at | BORA | DIS3 | 6.0 | 5.1 | 0.00 | 3.1 | 2.6E−03 |
| 3 | A.209971_x_at | A.217736_s_at | AIMP2 | EIF2AK1 | 8.0 | 9.2 | −0.81 | 6.2 | 3.5E−04 |
| 4 | A.217304_at | B.227304_at | SHMT1 | SMCR8 | 5.4 | 6.8 | 0.38 | 4.1 | 4.4E−03 |
| 5 | A.209690_s_at | A.208996_s_at | DOK4 | POLR2C | 4.4 | 7.8 | 0.00 | 6.2 | 1.0E−02 |
| 6 | B.228019_s_at | B.226521_s_at | MRPS18C | FAM175A | 8.4 | 6.7 | −0.65 | 7.2 | 9.4E−03 |
| 7 | A.204925_at | A.209154_at | CTNS | TAX1BP3 | 6.1 | 9.3 | 0.00 | 7.1 | 4.8E−02 |
| 8 | B.234464_s_at | B.234812_at | EME1 | LRRC59 | 7.5 | 3.9 | 0.00 | 7.2 | 8.5E−03 |
| 9 | B.226481_at | A.202689_at | VPRBP | RBM15B | 8.4 | 7.7 | 0.00 | 2.2 | 7.4E−04 |
| 10 | A.209510_at | B.223231_at | RNF139 | TATDN1 | 7.9 | 7.5 | −0.81 | 7.2 | 1.7E−02 |
| 11 | A.201139_s_at | A.221570_s_at | SSB | METTL5 | 7.3 | 7.9 | 0.00 | 4.1 | 3.1E−02 |
| 12 | B.222761_at | A.219479_at | BIVM | KDELC1 | 8.1 | 6.0 | 0.16 | 7.1 | 6.4E−03 |

TABLE 7E
The optimal SAGC classification parameters for breast cancer patients
with Grade 1 breast tumors.

| | | | | | | | | | 2-D |
|---|---|---|---|---|---|---|---|---|---|
| | | | | | | | | | RDDg |
| | Affymetrix | Affymetrix | | | | | | | Wald |
| | probeset for | probeset for | Gene | Gene | cut- | cut- | | | p- |
| pair | gene 1 | gene 2 | symbol 1 | symbol 2 | off1 | off2 | beta1 | design | value |
| 1 | B.232348_at | A.202679_at | C18orf8 | NPC1 | 4.4 | 7.4 | 0.16 | 2.2 | 3.5E−02 |
| 2 | A.219544_at | A.218362_s_at | BORA | DIS3 | 6.3 | 5.6 | 0.00 | 3.1 | 9.0E−04 |
| 3 | A.209971_x_at | A.217736_s_at | AIMP2 | EIF2AK1 | 8.2 | 9.1 | 0.00 | 2.2 | 6.6E−03 |
| 4 | A.217304_at | B.227304_at | SHMT1 | SMCR8 | 5.5 | 6.6 | 0.00 | 1.2 | 1.5E−02 |
| 5 | A.209690_s_at | A.208996_s_at | DOK4 | POLR2C | 5.0 | 7.9 | −0.16 | 6.2 | 5.1E−04 |
| 6 | B.228019_s_at | B.226521_s_at | MRPS18C | FAM175A | 8.3 | 7.0 | −0.51 | 7.2 | 1.2E−02 |
| 7 | A.204925_at | A.209154_at | CTNS | TAX1BP3 | 6.5 | 8.6 | 0.00 | 3.1 | 3.1E−03 |
| 8 | B.234464_s_at | B.234812_at | EME1 | LRRC59 | 7.2 | 4.9 | 0.81 | 1.1 | 1.5E−03 |
| 9 | B.226481_at | A.202689_at | VPRBP | RBM15B | 8.2 | 7.6 | −1.00 | 3.1 | 1.9E−04 |
| 10 | A.209510_at | B.223231_at | RNF139 | TATDN1 | 7.8 | 7.9 | 0.00 | 3.1 | 2.2E−03 |
| 11 | A.201139_s_at | A.221570_s_at | SSB | METTL5 | 7.3 | 8.0 | 0.00 | 5.1 | 2.9E−03 |
| 12 | B.222761_at | A.219479_at | BIVM | KDELC1 | 8.1 | 5.5 | 0.27 | 1.1 | 2.4E−03 |

TABLE 7F
The optimal SAGC classification parameters for breast cancer patients
with ER “−” breast tumors.

| | | | | | | | | | 2D |
|---|---|---|---|---|---|---|---|---|---|
| | | | | | | | | | RDDg |
| | Affymetrix | Affymetrix | | | | | | | Wald |
| | probeset for | probeset for | Gene | Gene | cut- | cut- | | | p- |
| pair | gene 1 | gene 2 | symbol 1 | symbol 2 | off1 | off2 | beta1 | design | value |
| 1 | B.232348_at | A.202679_at | C18orf8 | NPC1 | 3.7 | 7.5 | 0.51 | 7.1 | 3.9E−04 |
| 2 | A.219544_at | A.218362_s_at | BORA | DIS3 | 6.6 | 4.8 | 0.00 | 4.1 | 5.4E−04 |
| 3 | A.209971_x_at | A.217736_s_at | AIMP2 | EIF2AK1 | 8.2 | 9.6 | 0.65 | 6.2 | 5.4E−04 |
| 4 | A.217304_at | B.227304_at | SHMT1 | SMCR8 | 5.0 | 6.7 | −0.81 | 7.2 | 2.1E−02 |
| 5 | A.209690_s_at | A.208996_s_at | DOK4 | POLR2C | 5.4 | 8.0 | 0.65 | 7.2 | 3.1E−03 |
| 6 | B.228019_s_at | B.226521_s_at | MRPS18C | FAM175A | 8.0 | 6.6 | −0.27 | 6.1 | 9.6E−03 |
| 7 | A.204925_at | A.209154_at | CTNS | TAX1BP3 | 6.1 | 9.2 | 0.65 | 6.2 | 1.2E−03 |
| 8 | B.234464_s_at | B.234812_at | EME1 | LRRC59 | 8.0 | 4.3 | 0.00 | 5.2 | 3.2E−03 |
| 9 | B.226481_at | A.202689_at | VPRBP | RBM15B | 7.9 | 7.8 | 0.00 | 7.1 | 2.2E−02 |
| 10 | A.209510_at | B.223231_at | RNF139 | TATDN1 | 8.6 | 6.9 | 0.00 | 4.1 | 2.9E−03 |
| 11 | A.201139_s_at | A.221570_s_at | SSB | METTL5 | 7.0 | 7.0 | 0.00 | 6.1 | 1.0E−02 |
| 12 | B.222761_at | A.219479_at | BIVM | KDELC1 | 9.0 | 6.4 | −0.27 | 6.2 | 1.3E−02 |

TABLE 7G
The optimal SAGC classification parameters for breast cancer patients
with basal-like Grade 3 breast tumors.

| | | | | | | | | | 2D |
|---|---|---|---|---|---|---|---|---|---|
| | | | | | | | | | RDDg |
| | Affymetrix | Affymetrix | | | | | | | Wald |
| | probeset for | probeset for | Gene | Gene | cut- | cut- | | | p- |
| pair | gene 1 | gene 2 | symbol 1 | symbol 2 | off1 | off2 | beta1 | design | value |
| 1 | B.232348_at | A.202679_at | C18orf8 | NPC1 | 3.7 | 8.4 | 0.00 | 1.2 | 5.9E−03 |
| 2 | A.219544_at | A.218362_s_at | BORA | DIS3 | 7.4 | 5.5 | 0.05 | 2.2 | 1.4E−02 |
| 3 | A.209971_x_at | A.217736_s_at | AIMP2 | EIF2AK1 | 8.8 | 9.1 | −0.16 | 7.2 | 2.9E−03 |
| 4 | A.217304_at | B.227304_at | SHMT1 | SMCR8 | 5.3 | 6.8 | −0.51 | 1.2 | 8.3E−03 |
| 5 | A.209690_s_at | A.208996_s_at | DOK4 | POLR2C | 5.5 | 8.2 | 0.65 | 6.2 | 1.0E−02 |
| 6 | B.228019_s_at | B.226521_s_at | MRPS18C | FAM175A | 8.2 | 7.2 | −0.27 | 7.1 | 6.9E−03 |
| 7 | A.204925_at | A.209154_at | CTNS | TAX1BP3 | 6.4 | 9.5 | −0.16 | 6.2 | 1.0E−02 |
| 8 | B.234464_s_at | B.234812_at | EME1 | LRRC59 | 8.1 | 4.5 | 0.27 | 4.1 | 3.4E−03 |
| 9 | B.226481_at | A.202689_at | VPRBP | RBM15B | 8.6 | 8.2 | 0.00 | 7.2 | 1.2E−02 |
| 10 | A.209510_at | B.223231_at | RNF139 | TATDN1 | 7.7 | 8.5 | −0.51 | 5.1 | 1.5E−02 |
| 11 | A.201139_s_at | A.221570_s_at | SSB | METTL5 | 7.7 | 8.5 | −0.65 | 5.1 | 1.9E−02 |
| 12 | B.222761_at | A.219479_at | BIVM | KDELC1 | 8.8 | 5.7 | 0.00 | 7.2 | 7.9E−03 |

TABLE 7H
The optimal SAGC classification parameters for breast cancer patients
with Luminal A breast tumors.

| | | | | | | | | | 2D |
|---|---|---|---|---|---|---|---|---|---|
| | | | | | | | | | RDDg |
| | Affymetrix | Affymetrix | | | | | | | Wald |
| | probeset for | probeset for | Gene | Gene | cut- | cut- | | | p- |
| pair | gene 1 | gene 2 | symbol 1 | symbol 2 | off1 | off2 | beta1 | design | value |
| 1 | B.232348_at | A.202679_at | C18orf8 | NPC1 | 3.4 | 6.8 | −0.65 | 5.1 | 1.1E−02 |
| 2 | A.219544_at | A.218362_s_at | BORA | DIS3 | 6.2 | 6.1 | 0.00 | 2.2 | 4.8E−05 |
| 3 | A.209971_x_at | A.217736_s_at | AIMP2 | EIF2AK1 | 8.0 | 9.6 | 0.00 | 6.2 | 7.3E−03 |
| 4 | A.217304_at | B.227304_at | SHMT1 | SMCR8 | 5.3 | 6.6 | 0.00 | 2.2 | 3.7E−03 |
| 5 | A.209690_s_at | A.208996_s_at | DOK4 | POLR2C | 5.0 | 7.6 | −0.27 | 1.1 | 5.9E−03 |
| 6 | B.228019_s_at | B.226521_s_at | MRPS18C | FAM175A | 7.3 | 7.2 | 0.00 | 6.1 | 1.8E−02 |
| 7 | A.204925_at | A.209154_at | CTNS | TAX1BP3 | 6.4 | 9.3 | 0.27 | 1.1 | 2.3E−03 |
| 8 | B.234464_s_at | B.234812_at | EME1 | LRRC59 | 7.9 | 4.5 | −0.16 | 2.2 | 1.1E−04 |
| 9 | B.226481_at | A.202689_at | VPRBP | RBM15B | 8.2 | 7.2 | 0.16 | 7.2 | 1.7E−02 |
| 10 | A.209510_at | B.223231_at | RNF139 | TATDN1 | 7.8 | 7.5 | 0.65 | 5.1 | 1.5E−04 |
| 11 | A.201139_s_at | A.221570_s_at | SSB | METTL5 | 6.8 | 8.5 | 0.00 | 2.2 | 1.7E−02 |
| 12 | B.222761_at | A.219479_at | BIVM | KDELC1 | 8.7 | 5.3 | −0.05 | 6.1 | 1.6E−02 |

TABLE 7I
The optimal SAGC classification parameters for breast cancer patients with
ER“+”, LN“−”, PgR“+” breast tumors with size <=2 cm on the moment of curative
surgery who usually do not receive any systemic treatment.

| | | | | | | | | | 2D |
|---|---|---|---|---|---|---|---|---|---|
| | | | | | | | | | RDDg |
| | Affymetrix | Affymetrix | | | | | | | Wald |
| | probeset for | probeset for | Gene | Gene | cut- | cut- | | | p- |
| pair | gene 1 | gene 2 | symbol 1 | symbol 2 | off1 | off2 | beta1 | design | value |
| 1 | B.232348_at | A.202679_at | C18orf8 | NPC1 | 3.8 | 7.7 | 0.00 | 6.2 | 4.4E−03 |
| 2 | A.219544_at | A.218362_s_at | BORA | DIS3 | 6.2 | 5.8 | 0.27 | 7.2 | 1.4E−02 |
| 3 | A.209971_x_at | A.217736_s_at | AIMP2 | EIF2AK1 | 8.3 | 9.1 | 0.05 | 2.2 | 7.0E−03 |
| 4 | A.217304_at | B.227304_at | SHMT1 | SMCR8 | 5.5 | 7.3 | −0.38 | 6.2 | 8.2E−03 |
| 5 | A.209690_s_at | A.208996_s_at | DOK4 | POLR2C | 4.9 | 7.7 | −0.81 | 5.1 | 7.1E−03 |
| 6 | B.228019_s_at | B.226521_s_at | MRPS18C | FAM175A | 8.5 | 6.8 | 0.00 | 7.2 | 9.3E−03 |
| 7 | A.204925_at | A.209154_at | CTNS | TAX1BP3 | 6.7 | 9.1 | 0.00 | 6.2 | 9.0E−03 |
| 8 | B.234464_s_at | B.234812_at | EME1 | LRRC59 | 7.5 | 4.9 | 0.51 | 1.1 | 4.3E−02 |
| 9 | B.226481_at | A.202689_at | VPRBP | RBM15B | 8.3 | 7.8 | 0.00 | 3.1 | 1.6E−03 |
| 10 | A.209510_at | B.223231_at | RNF139 | TATDN1 | 8.5 | 7.4 | 0.00 | 7.2 | 1.9E−02 |
| 11 | A.201139_s_at | A.221570_s_at | SSB | METTL5 | 7.3 | 8.0 | 0.27 | 1.1 | 2.7E−03 |
| 12 | B.222761_at | A.219479_at | BIVM | KDELC1 | 8.2 | 6.4 | 0.00 | 2.2 | 8.7E−04 |

TABLE 7J
The optimal SAGC classification parameters for colon cancer patients with
stage II tumors⁵.

| | | | | | | | | | 2D |
|---|---|---|---|---|---|---|---|---|---|
| | | | | | | | | | RDDg |
| | Affymetrix | Affymetrix | | | | | | | Wald |
| | probeset for | probeset for | Gene | Gene | cut- | cut- | | | p- |
| pair | for gene 1 | gene 2 | symbol 1 | symbol 2 | off1 | off2 | beta1 | design | value |
| 1 | B.232348_at | A.202679_at | C18orf8 | NPC1 | 1 | 280 | 0.00 | 5.1 | 5.5E−04 |
| 2 | A.219544_at | A.218362_s_at | BORA | DIS3 | 123 | 64 | −0.51 | 7.1 | 4.6E−03 |
| 3 | A.209971_x_at | A.217736_s_at | AIMP2 | EIF2AK1 | 500 | 888 | 0.00 | 6.1 | 7.5E−03 |
| 4 | A.217304_at | B.227304_at | SHMT1 | SMCR8 | 6 | 42 | −0.65 | 7.2 | 3.9E−02 |
| 5 | A.209690_s_at | A.208996_s_at | DOK4 | POLR2C | 51 | 457 | 0.65 | 5.1 | 1.5E−02 |
| 6 | B.228019_s_at | B.226521_s_at | MRPS18C | FAM175A | 333 | 76 | −0.81 | 7.2 | 2.3E−02 |
| 7 | A.204925_at | A.209154_at | CTNS | TAX1BP3 | 94 | 2043 | 0.05 | 7.2 | 8.9E−03 |
| 8 | B.234464_s_at | B.234812_at | EME1 | LRRC59 | 160 | 2 | 0.05 | 1.2 | 2.3E−04 |
| 9 | B.226481_at | A.202689_at | VPRBP | RBM15B | 234 | 85 | 0.00 | 7.1 | 4.0E−04 |
| 10 | A.209510_at | B.223231_at | RNF139 | TATDN1 | 379 | 478 | −0.38 | 7.2 | 2.5E−04 |
| 11 | A.201139_s_at | A.221570_s_at | SSB | METTL5 | 725 | 765 | 0.00 | 6.1 | 5.6E−03 |
| 12 | B.222761_at | A.219479_at | BIVM | KDELC1 | 102 | 149 | 0.00 | 6.2 | 7.8E−03 |

TABLE 7K
The optimal SAGC classification parameters for non-small cell lung
cancer patients tumors.⁶

| | | | | | | | | | 2D |
|---|---|---|---|---|---|---|---|---|---|
| | | | | | | | | | RDDg |
| | Affymetrix | Affymetrix | | | | | | | Wald |
| | probeset for | probeset for | Gene | Gene | cut- | cut- | | | p- |
| pair | gene 1 | gene 2 | symbol 1 | symbol 2 | off1 | off2 | beta1 | design | value |
| 1 | B.232348_at | A.202679_at | C18orf8 | NPC1 | −0.09 | −0.23 | 0.38 | 3.1 | 4.8E−04 |
| 2 | A.219544_at | A.218362_s_at | BORA | DIS3 | 1.22 | 0.01 | 0.38 | 1.2 | 5.5E−03 |
| 3 | A.209971_x_at | A.217736_s_at | AIMP2 | EIF2AK1 | 0.37 | 0.57 | 0.65 | 6.1 | 8.0E−03 |
| 4 | A.217304_at | B.227304_at | SHMT1 | SMCR8 | −0.25 | −1.03 | −0.16 | 6.1 | 7.8E−03 |
| 5 | A.209690_s_at | A.208996_s_at | DOK4 | POLR2C | −0.09 | 0.06 | 0.00 | 6.1 | 9.1E−04 |
| 6 | B.228019_s_at | B.226521_s_at | MRPS18C | FAM175A | 0.44 | 0.32 | 0.00 | 4.2 | 2.1E−03 |
| 7 | A.204925_at | A.209154_at | CTNS | TAX1BP3 | −0.30 | −0.65 | 0.65 | 5.1 | 1.1E−02 |
| 8 | B.234464_s_at | B.234812_at | EME1 | LRRC59 | 0.88 | −0.09 | 0.00 | 4.2 | 1.5E−02 |
| 9 | B.226481_at | A.202689_at | VPRBP | RBM15B | −0.37 | 0.21 | 0.00 | 7.1 | 9.7E−03 |
| 10 | A.209510_at | B.223231_at | RNF139 | TATDN1 | 0.06 | −0.34 | 0.00 | 1.2 | 1.7E−02 |
| 11 | A.201139_s_at | A.221570_s_at | SSB | METTL5 | −0.17 | 0.51 | 0.27 | 2.2 | 6.3E−04 |
| 12 | B.222761_at | A.219479_at | BIVM | KDELC1 | −0.72 | 0.33 | 0.27 | 6.2 | 3.1E−02 |

TABLE 7L
The optimal SAGC classification parameters for total unselected groups of breast tumors.

| | | | | | | 1D DDg Wald | | | | | |
|---|---|---|---|---|---|---|---|---|---|---|---|
| | | | | | 1D DDg Wald | p-value for | | | | | 2D |
| | Affymetrix | Affymetrix | | | p-value for | individual | | | | | RDDg |
| | probeset for | probeset for | Gene | Gene | individual gene 1 | gene 2 | cut- | cut- | | | Wald p- |
| pair | gene 1 | gene 2 | symbol 1 | symbol 2 | in the pair | in the pair | off1 | off2 | beta1 | design | value |
| | | | | | Total group, Stockholm cohort | | | | | | |
| 1 | B.232348_at | A.202679_at | C18orf8 | NPC1 | 3.9E−02 | 1.6E−02 | 3.8 | 7.8 | 0.27 | 2.2 | 1.3E−02 |
| 2 | A.219544_at | A.218362_s_at | BORA | DIS3 | 8.0E−03 | 3.0E−04 | 6.7 | 5.4 | −0.51 | 3.1 | 2.7E−07 |
| 3 | A.209971_x_at | A.217736_s_at | AIMP2 | EIF2AK1 | 1.6E−06 | 2.5E−04 | 8.2 | 9.1 | 0.16 | 7.2 | 1.1E−06 |
| 4 | A.217304_at | B.227304_at | SHMT1 | SMCR8 | 3.8E−02 | 1.3E−02 | 5.4 | 6.7 | 0.05 | 4.1 | 1.3E−03 |
| 5 | A.209690_s_at | A.208996_s_at | DOK4 | POLR2C | 1.6E−02 | 1.9E−05 | 5.8 | 8.1 | 0.05 | 5.1 | 1.1E−06 |
| 6 | B.228019_s_at | B.226521_s_at | MRPS18C | FAM175A | 2.3E−04 | 1.1E−01 | 8.2 | 6.7 | −0.51 | 7.2 | 2.3E−05 |
| 7 | A.204925_at | A.209154_at | CTNS | TAX1BP3 | 3.1E−01 | 5.2E−03 | 6.5 | 9.3 | 0.27 | 2.2 | 4.7E−03 |
| 8 | B.234464_s_at | B.234812_at | EME1 | LRRC59 | 2.4E−03 | 1.2E−01 | 7.6 | 5.1 | 0.00 | 3.1 | 4.2E−04 |
| 9 | B.226481_at | A.202689_at | VPRBP | RBM15B | 1.4E−02 | 3.9E−02 | 8.2 | 7.7 | −1.00 | 2.2 | 8.4E−03 |
| 10 | A.209510_at | B.223231_at | RNF139 | TATDN1 | 7.0E−03 | 9.4E−03 | 7.5 | 8.2 | 0.51 | 2.2 | 6.6E−03 |
| 11 | A.201139_s_at | A.221570_s_at | SSB | METTL5 | 9.0E−02 | 2.5E−02 | 7.1 | 8.1 | −0.16 | 6.2 | 1.1E−02 |
| 12 | B.222761 at | A.219479_at | BIVM | KDELC1 | 6.0E−02 | 1.1E−01 | 7.8 | 5.7 | 0.00 | 2.1 | 1.4E−02 |
| | | | | | Total group, Uppsala cohort | | | | | | |
| 1 | B.232348_at | A.202679_at | C18orf8 | NPC1 | 3.2E−02 | 2.8E−03 | 3.8 | 7.7 | 0.27 | 2.2 | 5.8E−10 |
| 2 | A.219544_at | A.218362_s_at | BORA | DIS3 | 3.0E−03 | 1.4E−01 | 6.7 | 5.4 | −0.51 | 3.1 | 1.5E−03 |
| 3 | A.209971_x_at | A.217736_s_at | AIMP2 | EIF2AK1 | 5.5E−03 | 2.7E−02 | 8.3 | 9.1 | 0.16 | 7.2 | 3.0E−03 |
| 4 | A.217304_at | B.227304_at | SHMT1 | SMCR8 | 6.1E−02 | 1.0E−02 | 5.4 | 6.7 | 0.05 | 4.1 | 7.1E−06 |
| 5 | A.209690_s_at | A.208996_s_at | DOK4 | POLR2C | 4.5E−02 | 5.1E−02 | 5.8 | 8.1 | 0.05 | 5.1 | 1.6E−02 |
| 6 | B.228019_s_at | B.226521_s_at | MRPS18C | FAM175A | 7.1E−02 | 2.9E−02 | 8.2 | 6.7 | −0.51 | 7.2 | 2.3E−03 |
| 7 | A.204925_at | A.209154_at | CTNS | TAX1BP3 | 2.3E−02 | 2.2E−03 | 6.5 | 9.3 | 0.27 | 2.2 | 2.6E−05 |
| 8 | B.234464_s_at | B.234812_at | EME1 | LRRC59 | 2.1E−03 | 6.7E−02 | 7.6 | 5.1 | 0.00 | 3.1 | 5.4E−04 |
| 9 | B.226481_at | A.202689_at | VPRBP | RBM15B | 3.0E−03 | 3.4E−02 | 8.2 | 7.7 | −1.00 | 2.2 | 2.7E−04 |
| 10 | A.209510_at | B.223231_at | RNF139 | TATDN1 | 4.1E−02 | 1.9E−02 | 7.5 | 8.2 | 0.51 | 2.2 | 4.2E−03 |
| 11 | A.201139_s_at | A.221570_s_at | SSB | METTL5 | 2.1E−02 | 1.3E−02 | 7.1 | 8.1 | −0.16 | 6.2 | 9.0E−03 |
| 12 | B.222761_at | A.219479_at | BIVM | KDELC1 | 1.4E−01 | 7.7E−02 | 7.8 | 5.7 | 0.00 | 2.1 | 4.5E−02 |

TABLE 8
Literature analysis for the genes composing 12 survival-significant
synergistic ccSAGPs in two breast cancer cohorts (Stockholm and Uppsala cohorts).

| RefSeq | | Associations | | RefSeq | | Association | |
|---|---|---|---|---|---|---|---|
| gene | Gene | with | | gene | Gene | with | |
| symbol | description | cancer(s) | Ref | symbol | description | cancer (s) | Ref |
| C18orf8 | chromosome | | | NPC1 | Niemann- | NPC1 | [83] |
| | 18 | | | | Pick | activity is | |
| | open | | | | disease, | associated | |
| | reading | | | | type C1 | with the | |
| | frame 8 | | | | precursor | emergence | |
| | | | | | | of multidrug | |
| | | | | | | resistance | |
| | | | | | | of HL-60 | |
| | | | | | | cancer cell | |
| | | | | | | line | |
| C13orf34 | aurora | Radiation | [84, 85, | DIS3 | DIS3 mitotic | Differentially | [87, 88] |
| | borealis | sensitivity in | 86] | | control | expressed | |
| | (BORA) | cancer; | | | homolog | in colorectal | |
| | | breast | | | (S. cerevisiae) | carcinoma; | |
| | | cancer; | | | | control of | |
| | | activator of | | | | mitosis | |
| | | the protein | | | | | |
| | | kinase | | | | | |
| | | Aurora A; | | | | | |
| | | control of | | | | | |
| | | mitosis | | | | | |
| AIMP2 | Aminoacyl | Tumor | [89, 90] | EIF2AK1 | Eukaryotic | | |
| | tRNA | suppressor | | | translation | | |
| | synthetase | in lung and | | | initiation | | |
| | complex- | ovarian | | | factor 2- | | |
| | interacting | cancer | | | alpha | | |
| | multifunctional | | | | kinase 1 | | |
| | protein 2 | | | | | | |
| SHMT1 | serine | Associations | [91] | SMCR8 | Smith- | | |
| | hydroxy | with rectal | | | Magenis | | |
| | methyl- | and intestinal | | | syndrome | | |
| | transferase 1 | cancers | | | chromosome | | |
| | | | | | region, | | |
| | | | | | candidate 8 | | |
| DOK4 | docking | Altered | [92] | POLR2C | DNA | the | [93] |
| | protein 4 | expression in | | | directed | POLR2C | |
| | | clear cell | | | RNA | rs4937 | |
| | | renal cell | | | polymerase | polymorphism | |
| | | carcinoma | | | II | is | |
| | | | | | polypeptide C | associated | |
| | | | | | | with the | |
| | | | | | | response to | |
| | | | | | | the | |
| | | | | | | oxaliplatin- | |
| | | | | | | based | |
| | | | | | | chemotherapy | |
| MRPS18C | Mitochondrial | | | FAM175A | Family with | BRCA1-A | [94] |
| | ribosomal | | | | sequence | complex | |
| | protein | | | | similarity | subunit; | |
| | S18C | | | | 175 | potential | |
| | | | | | | marker of | |
| | | | | | | platinum- | |
| | | | | | | gemcitabine | |
| | | | | | | chemotherapy | |
| CTNS | Cystinosin, | | | TAX1BP3 | Tax1 | Oncogenic | [95] |
| | lysosomal | | | | (human T- | protein, | |
| | cystine | | | | cell | potential | |
| | transporter | | | | leukemia | marker of | |
| | | | | | virus type I) | invasive | |
| | | | | | binding | breast | |
| | | | | | protein 3 | tumors | |
| EME1 | Essential | Potential | [96] | LRRC59 | Leucine rich | | |
| | meiotic | sensitivity | | | repeat | | |
| | endonuclease 1 | marker for | | | containing | | |
| | homolog 1 | cisplatin- | | | 59 | | |
| | | based | | | | | |
| | | chemotherapy | | | | | |
| | | in various | | | | | |
| | | tumors | | | | | |
| VPRBP | Vpr (HIV- | Inhibitor of | [97] | RBM15B | RNA | | |
| | 1) | p53 target | | | binding | | |
| | binding | genes | | | motif | | |
| | protein | | | | protein 15B | | |
| RNF139 | Ring | Putative | [98] | TATDN1 | TatD | Fusion | [99] |
| | finger | tumor | | | DNase | gene in | |
| | protein | suppressor in | | | domain | breast | |
| | 139 | kidney | | | containing 1 | cancer | |
| | | cancer | | | | | |
| SSB | Sjogren | | | METTL5 | Methyltransferase | | |
| | syndrome | | | | like 5 | | |
| | antigen B | | | | | | |
| BIVM | basic, | | | KDELC1 | KDEL (Lys- | | |
| | immunoglobulin- | | | | Asp-Glu- | | |
| | like | | | | Leu) | | |
| | variable | | | | containing 1 | | |
| | motif | | | | | | |

TABLE 9
Oligoprimers and TaqMan probes used for strand-specific QRT-PCR in nine
3S-ccSAGPs (eighteen genes) and two internal controls.

| Symbol/ | | | Sequence of | | |
|---|---|---|---|---|---|
| tran- | | | oligoprimers used in QRT-PCR | | |
| script | | Affymetrix | Forward | Reverse | |
| ID. | Strand | probset | primer | primer | TaqMan probe |
| BORA | + | A.219544_ | 5′-CTTTTCAATGCAGCAGTCCA-3′ | 5′-TTCCAGGAGCATTTCTGTTG-3′ | FAM- |
| | | at | | | TGCCTCTGTCAGAATCA |
| | | | | | AAGACTAAGC-TAMRA |
| DIS3 | - | A218362_ | 5′-ATGTTACCATGGCTGTGTCC-3′ | 5′-ACCTAGCCGGCAAACTTTTT-3′ | FAM- |
| | | s_at | | | CCACCCCCGCCAACCAT |
| | | | | | TT-TAMRA |
| AIMP2 | + | A.209971_ | 5′-GGGAACATTGCACGTTTCTT-3′ | 5′-AGAGTTCATGGAGCGGAAAA-3′ | FAM- |
| | | x_at | | | TGCTGTCAACGCAACCC |
| | | | | | TTA-TAMRA |
| EIF2AK1 | - | A.217736_ | 5′-GCCTGCACATACTCACTGGA-3′ | 5′-CTTTGCAAGGGTGTGGTTTT-3′ | FAM- |
| | | s_at | | | CCAGTCTTCCTGCCCCT |
| | | | | | TTC-TAMRA |
| POLR2C | + | A.208996_ | 5′-GTTGAGCTTCTTGGCAGGAC-3′ | 5′-AGGTGCACTGGAAACACTCC-3′ | FAM- |
| | | s_at | | | ACCAGGGATGCAGTGGT |
| | | | | | GTT-TAMRA |
| DOK4 | - | A.209690_ | 5′-CAAGGGCACGGAACATTACT-3′ | 5′-GCTTTGGCTTTAGCAGGATG-3′ | FAM- |
| | | s_at | | | CAGTGCCTACTGGCACC |
| | | | | | ACA-TAMRA |
| MRPS18C | + | B.228019_ | 5′-ACTTGGTAACGGCTGCTGTC-3′ | 5′-TGCCTTCCATAAATGCATCC-3′ | FAM- |
| | | s_at | | | CTCACACGGTGCTTTGG |
| | | | | | AGAA-TAMRA |
| FAM175A | - | B.226521_ | 5′-TGAGCTATTTGGGGATCAGA-3′ | 5′-TGACTCAAACCAACCTTTGGA-3′ | FAM- |
| | | s_at | | | TGGACATTGGTATGAAG |
| | | | | | GGGA-TAMRA |
| CTNS | + | A.204925_ | 5′-AGATTAGCCCCATCTGAGCA-3′ | 5′-CAGGCCAGAAATTGGTGAGT-3′ | FAM- |
| | | at | | | CCCAGCATCTGGAGTAC |
| | | | | | AGGA-TAMRA |
| TAX1BP3 | - | A.209154_ | 5′-CACCTATCAGGGCAGCTCTC-3′ | 5′-TAAGGCTTGTGCACTTGCTG-3′ | FAM- |
| | | at | | | TTCCTCTTTGCCTGTGG |
| | | | | | GTTTA-TAMRA |
| EME1 | + | B.234464_ | 5′-TTGAAGTCGTGAGTGAAGCTG- | 5′-TTGGTACAAATGCTTTATTGAA | FAM- |
| | | s_at | 3′ | AC-3′ | CTTGACTGGGCTCAGCC |
| | | | | | ACTGA-TAMRA |
| LRRC59 | - | B.234812_ | 5′-GGCCAAAAATTTGCTTTCCT-3′ | 5′-TGCAGGCTGATATCATAGGC-3′ | FAM- |
| | | at | | | TGCTTATTACATTGTGA |
| | | | | | TCTGACCTG-TAMRA |
| RBM15B | + | A.202689_ | 5′-AGGACATTGGGCAAGCTAGA-3′ | 5′-GTTTTGGTGGTGGACAGGAC-3′ | FAM- |
| | | at | | | CCTGTCTTTCCCAGCGT |
| | | | | | CAT-TAMRA |
| VPRBP | - | B.226481_ | 5′-TGCCTCATATGCTGTCAGGT-3′ | 5′-TTTCCACAATGGGGAGAAAG-3′ | FAM- |
| | | at | | | TTTCCTTCGTGCCAGAG |
| | | | | | GTG-TAMRA |
| SSB | + | A.201139_ | 5′-GTGCAACTGGACCTGTGAAA-3′ | 5′-CAGGTGGACATTGAAGTGGA-3′ | FAM- |
| | | s_at | | | ACTTTTGTTTGCGGGGC |
| | | | | | TTT-TAMRA |
| METTL5 | - | A.221570_ | 5′-GCATCGGAACTGCAATGTTA-3′ | 5′-GTCCCAAAGGGAGGATTCAT-3′ | FAM- |
| | | s_at | | | CATGGTTCAATGTGATG |
| | | | | | TGTGC-TAMRA |
| BIVM | + | B.222761_ | 5′-TTTTCTTCTGCTGCCAACCT-3′ | 5′-CCTTTTCCTTGAGGTGTATTGC-3′ | FAM- |
| | | at | | | GGGTCTTGTTCCAGGAC |
| | | | | | TCAAA-TAMRA |
| KDELC1 | - | A.2219479_ | 5′-GTGCTGAAGCAGGATTCCAT-3′ | 5′-TGTCATCGCCCATGAGATTA-3′ | FAM- |
| | | at | | | CTGCAGCCCTGGAAACA |
| | | | | | CT-TAMRA |
| ACTB | - | NA | CAGCCATGTACGTTGCTATCCAGG | AGGTCCAGACGCAGGATGGCATG | FAM- |
| | | | | | ACTGGCATCGTGATGGA |
| | | | | | CTC-TAMRA |
| TBP | + | NA | TGCCCGAAACGCCGAATATAATC | GTCTGGACTGTTCTTCACTCTTGG | FAM- |
| | | | | | CAAGCGGTTTGCTGCGG |
| | | | | | TAATC-TAMRA |

TABLE 10
Twenty seven proteasomal and twenty five spliceosomal genes identified in total groups
of BC patients using SAGC (see Tables 6 and 11). *http://mgc.nci.nih.gov/

| | | | | Mammalian | | Functional Annot. terms |
|---|---|---|---|---|---|---|
| Gene | Gene | Affymetrix | | Gene Collection | | under the category |
| # | symbol | probeset | Gene description | cDNA clone ID* | cytoband | KEGG_PATHWAY |
| 1 | SHFM1 | 202276_at | split hand/foot malformation | BC032782 | 7q21.3 | hsa03050:Proteasome |
| | | | (ectrodactyly) type 1 | | | |
| 2 | PSMA7 | 201114_x_at | proteasome subunit, alpha type, 7 | BC004427 | 20q13.33 | hsa03050:Proteasome |
| 3 | PSMB5 | 208799_at | proteasome subunit, beta type, 5 | BC057840 | 14q11.2 | hsa03050:Proteasome |
| 4 | PSMB4 | 202243_s_at | proteasome subunit, beta type, 4 | na | 1q21 | hsa03050:Proteasome |
| 5 | PSMB7 | 200786_at | proteasome subunit, beta type, 7 | BC000509 | 9q34.11-q34.12 | hsa03050:Proteasome |
| 6 | PSMB6 | 208827_at | proteasome subunit, beta type, 6 | BC000835 | 17p13 | hsa03050:Proteasome |
| 7 | PSMB1 | 200876_s_at | proteasome subunit, beta type, 1 | BC000508 | 6q27 | hsa03050:Proteasome |
| 8 | PSMB2 | 200039_s_at | proteasome subunit, beta type, 2 | BC101836 | 1p34.2 | hsa03050:Proteasome |
| 9 | PSMD1 | 201198_s_at | proteasome 26S subunit, non- | BC094720 | 2q37.1 | hsa03050:Proteasome |
| | | | ATPase, 1 | | | |
| 10 | PSMD2 | 200830_at | proteasome 26S subunit, non- | BC007897 | 3q27.1 | hsa03050:Proteasome |
| | | | ATPase, 2 | | | |
| 11 | PSMD4 | 200882_s_at | proteasome 26S subunit, non- | BC002365 | 1q21.3 | hsa03050:Proteasome |
| | | | ATPase, 4 | | | |
| 12 | PSMD7 | 201705_at | proteasome 26S subunit, non- | BC012606 | 16q22.3 | hsa03050:Proteasome |
| | | | ATPase, 7 | | | |
| 13 | PSMA2 | 201317_s_at | proteasome subunit, alpha type, 2 | BC047697 | 7p13 | hsa03050:Proteasome |
| 14 | PSMA1 | 210759_s_at | proteasome subunit, alpha type, 1 | BC015356 | 11p15.1 | hsa03050:Proteasome |
| 15 | PSMD14 | 212296_at | proteasome 26S subunit, non- | BC066336 | 2q24.2 | hsa03050:Proteasome |
| | | | ATPase, 14 | | | |
| 16 | PSMA6 | 208805_at | proteasome subunit, alpha type, 6 | BC002979 | 14q13 | hsa03050:Proteasome |
| 17 | PSMD12 | 202352_s_at | proteasome 26S subunit, non- | BC019062 | 17q24.2 | hsa03050:Proteasome |
| | | | ATPase, 12 | | | |
| 18 | PSMA5 | 201274_at | proteasome subunit, alpha type, 5 | BC103751 | 1p13 | hsa03050:Proteasome |
| 19 | PSMD11 | 208777_s_at | proteasome 26S subunit, non- | BC000437 | 17q11.2 | hsa03050:Proteasome |
| | | | ATPase, 11 | | | |
| 20 | PSMC3 | 201267_s_at | proteasome 26S subunit, ATPase, 3 | BC008713 | 11p11.2 | hsa03050:Proteasome |
| 21 | PSMA4 | 203396_at | proteasome subunit, alpha type, 4 | BC005361 | 15q25.1 | hsa03050:Proteasome |
| 22 | PSMC2 | 201068_s_at | proteasome 26S subunit, ATPase, 2 | BC002589 | 7q22.1-q22.3 | hsa03050:Proteasome |
| 23 | PSMC1 | 204219_s_at | proteasome 26S subunit, ATPase, 1 | BC000512 | 14q32.11 | hsa03050:Proteasome |
| 24 | PSMA3 | 201532_at | proteasome subunit, alpha type, 3 | BC005265 | 14q23 | hsa03050:Proteasome |
| 25 | POMP | 217769_s_at | proteasome maturation protein | BC003390 | 13q12.3 | hsa03050:Proteasome |
| 26 | PSME3 | 209853_s_at | proteasome activator subunit 3 | BC008020 | 17q21 | hsa03050:Proteasome |
| 27 | PSME4 | 212219_at | proteasome activator subunit 4 | BC112169 | 2p16.2 | hsa03050:Proteasome |
| 1 | NCBP1 | 209520_s_at | nuclear cap binding protein | BC001450 | 9q34.1 | hsa03040:Spliceosome |
| | | | subunit 1, 80 kDa | | | |
| 2 | NHP2L1 | 201077_s_at | NHP2 non-histone chromosome | BC005358 | 22q13 | hsa03040:Spliceosome |
| | | | protein 2-like 1 | | | |
| 3 | PPIL1 | 222500_at | peptidylprolyl isomerase | BC003048 | 6p21.1 | hsa03040:Spliceosome |
| | | | (cyclophilin)-like 1 | | | |
| 4 | LSM7 | 204559_s_at | LSM7 homolog, U6 small nuclear | BC018621 | 19p13.3 | hsa03040:Spliceosome |
| | | | RNA associated | | | |
| 5 | SNRPD1 | 202690_s_at | small nuclear ribonucleoprotein | BC001721 | 18q11.2 | hsa03040:Spliceosome |
| | | | D1 polypeptide 16 kDa | | | |
| 6 | SNRPD2 | 200826_at | small nuclear ribonucleoprotein | BC000486 | 19q13.2 | hsa03040:Spliceosome |
| | | | D2 polypeptide 16.5 kDa | | | |
| 7 | SF3B5 | 221263_s_at | splicing factor 3b, subunit 5, | BC000198 | 6q24.2 | hsa03040:Spliceosome |
| | | | 10 kDa | | | |
| 8 | SF3B3 | 200687_s_at | splicing factor 3b, subunit 3, | BC003146 | 16q22.1 | hsa03040:Spliceosome |
| | | | 130 kDa | | | |
| 9 | HNRNPA3 | 211930_at | heterogeneous nuclear | BC064494 | 2q31.2 | hsa03040:Spliceosome |
| | | | ribonucleoprotein A3 | | | |
| 10 | HNRNPK | 200775_s_at | heterogeneous nuclear | BC000355 | 9q21.32-q21.33 | hsa03040:Spliceosome |
| | | | ribonucleoprotein K | | | |
| 11 | RBM8A | 222443_s_at | RNA binding motif protein 8A | BC017088 | 1q12 | hsa03040:Spliceosome |
| 12 | USP39 | 217829_s_at | ubiquitin specific peptidase 39 | BC067273 | 2p11.2 | hsa03040:Spliceosome |
| 13 | LSM4 | 202737_s_at | LSM4 homolog, U6 small nuclear | BC000387 | 19p13.11 | hsa03040:Spliceosome |
| | | | RNA associated | | | |
| 14 | LSM3 | 202209_at | LSM3 homolog, U6 small nuclear | BC007055 | 3p25.1 | hsa03040:Spliceosome |
| | | | RNA associated | | | |
| 15 | SNRPA1 | 215722_s_at | small nuclear ribonucleoprotein | BC022816 | 15q26.3 | hsa03040:Spliceosome |
| | | | polypeptide A′ | | | |
| 16 | EFTUD2 | 222398_s_at | elongation factor Tu GTP binding | BC002360 | 17q21.31 | hsa03040:Spliceosome |
| | | | domain containing 2 | | | |
| 17 | PRPF18 | 221547_at | PRP18 pre-mRNA processing | BC000794 | 10p13 | hsa03040:Spliceosome |
| | | | factor 18 homolog | | | |
| 18 | EIF4A3 | 201303_at | eukaryotic translation initiation | BC004386 | 17q25.3 | hsa03040:Spliceosome |
| | | | factor 4A3 | | | |
| 19 | SNRPB | 213175_s_at | small nuclear ribonucleoprotein | BC080516 | 20p13 | hsa03040:Spliceosome |
| | | | polypeptides B and B1 | | | |
| 20 | SNRPA | 201770_at | small nuclear ribonucleoprotein | BC000405 | 19q13.1 | hsa03040:Spliceosome |
| | | | polypeptide A | | | |
| 21 | SNRPC | 201342_at | small nuclear ribonucleoprotein | BC121082 | 6p21.31 | hsa03040:Spliceosome |
| | | | polypeptide C | | | |
| 22 | SNRNP27 | 212438_at | small nuclear ribonucleoprotein | BC017890 | 2p13.3 | hsa03040:Spliceosome |
| | | | 27 kDa (U4/U6.U5) | | | |
| 23 | PUF60 | 209899_s_at | poly-U binding splicing factor | BC008875 | 8q24.3 | hsa03040:Spliceosome |
| | | | 60 KDa | | | |
| 24 | SNRPG | 205644_s_at | small nuclear ribonucleoprotein | BC000070 | 2p13.3 | hsa03040:Spliceosome |
| | | | polypeptide G | | | |
| 25 | RBM17 | 224781_s_at | RNA binding motif protein 17 | BC007871 | 10p15.1 | hsa03040:Spliceosome |

TABLE 11
150 genes robustly upregulated in HR subgroups classified by the SAGC and belonging to significantly
enriched (overrepresented) biologically-related Functional Annotation terms and category
KEGG_PATHWAY (refer to Table 6). Rows in bold: genes represented in the Table 10.
*http://mgc.nci.nih.gov/

| | | | | | | | | | To which enriched |
|---|---|---|---|---|---|---|---|---|---|
| | | | | | | Measure of differential gene | | | biologically-related |
| | | | | | | expression in HR vs. LR | | | term/category the |
| | | | | Mammalian | | subgroups in a BC cohort | | | gene belongs |
| | | | | Gene | | Uppsala | Stockholm | Metadata | Functional Annot. |
| | | | | Collection | | cohort, t- | cohort, t- | cohort, | terms under the |
| Gene | Gene | Affymetrix | | cDNA | | test Q- | test Q- | t-test Q- | category |
| # | symbol | probeset | Gene description | clone ID* | cytoband | Value | Value | Value | KEGG_PATHWAY |
| 1 | SHFM1 | 202276_at | split hand/foot | BC032782 | 7q21.3 | 4.7E−06 | 8.7E−06 | 3.50E−16 | hsa03050:Proteasome |
| | | | malformation | | | | | | |
| | | | (ectrodactyly) type 1 | | | | | | |
| 2 | PSMA7 | 201114_x_at | proteasome subunit, | BC004427 | 20q13.33 | 4.7E−06 | 8.7E−06 | 3.50E−16 | hsa03050:Proteasome |
| | | | alpha type, 7 | | | | | | |
| 3 | PSMB5 | 208799_at | proteasome subunit, | BC057840 | 14q11.2 | 4.7E−06 | 4.0E−04 | 7.54E−13 | hsa03050:Proteasome |
| | | | beta type, 5 | | | | | | |
| 4 | PSMB4 | 202243_s_at | proteasome subunit, | na | 1q21 | 4.7E−06 | 8.7E−06 | 3.50E−16 | hsa03050:Proteasome |
| | | | beta type, 4 | | | | | | |
| 5 | PSMB7 | 200786_at | proteasome subunit, | BC000509 | 9834.11- | 4.7E−06 | 1.2E−04 | 6.54E−14 | hsa03050:Proteasome |
| | | | beta type, 7 | | q34.12 | | | | |
| 6 | PSMB6 | 208827_at | proteasome subunit, | BC000835 | 17p13 | 4.7E−06 | 1.6E−05 | 1.16E−15 | hsa03050:Proteasome |
| | | | beta type, 6 | | | | | | |
| 7 | PSMB1 | 200876_s_at | proteasome subunit, | BC000508 | 6q27 | 4.7E−06 | 8.7E−06 | 3.50E−16 | hsa03050:Proteasome |
| | | | beta type, 1 | | | | | | |
| 8 | PSMB2 | 200039_s_at | proteasome subunit, | BC101836 | 1p34.2 | 4.7E−06 | 8.7E−06 | 3.50E−16 | hsa03050:Proteasome |
| | | | beta type, 2 | | | | | | |
| 9 | PSMD1 | 201198_s_at | proteasome 26S | BC094720 | 2q37.1 | 4.7E−06 | 5.3E−04 | 1.31E−12 | hsa03050:Proteasome |
| | | | subunit, non-ATPase, 1 | | | | | | |
| 10 | PSMD2 | 200830_at | proteasome 26S | BC007897 | 3q27.1 | 4.7E−06 | 1.9E−04 | 1.70E−13 | hsa03050:Proteasome |
| | | | subunit, non-ATPase, 2 | | | | | | |
| 11 | PSMD4 | 200882_s_at | proteasome 26S | BC002365 | 1q21.3 | 4.7E−06 | 1.6E−05 | 1.16E−15 | hsa03050:Proteasome |
| | | | subunit, non-ATPase, 4 | | | | | | |
| 12 | PSMD7 | 201705_at | proteasome 26S | BC012606 | 16q22.3 | 4.7E−06 | 6.3E−04 | 1.87E−12 | hsa03050:Proteasome |
| | | | subunit, non-ATPase, 7 | | | | | | |
| 13 | PSMA2 | 201317_s_at | proteasome subunit, | BC047697 | 7p13 | 4.7E−06 | 6.4E−04 | 1.90E−12 | hsa03050:Proteasome |
| | | | alpha type, 2 | | | | | | |
| 14 | PSMA1 | 210759_s_at | proteasome subunit, | BC015356 | 11p15.1 | 4.7E−06 | 8.7E−06 | 3.50E−16 | hsa03050:Proteasome |
| | | | alpha type, 1 | | | | | | |
| 15 | PSMD14 | 212296_at | proteasome 26S | BC066336 | 2q24.2 | 4.7E−06 | 8.7E−06 | 3.50E−16 | hsa03050:Proteasome |
| | | | subunit, non-ATPase, | | | | | | |
| | | | 14 | | | | | | |
| 16 | PSMA6 | 208805_at | proteasome subunit, | BC002979 | 14q13 | 4.7E−06 | 2.8E−03 | 3.64E−11 | hsa03050:Proteasome |
| | | | alpha type, 6 | | | | | | |
| 17 | PSMD12 | 202352_s_at | proteasome 26S | BC019062 | 17q24.2 | 4.7E−06 | 8.7E−06 | 3.50E−16 | hsa03050:Proteasome |
| | | | subunit, non-ATPase, | | | | | | |
| | | | 12 | | | | | | |
| 18 | PSMA5 | 201274_at | proteasome subunit, | BC103751 | 1p13 | 4.7E−06 | 4.2E−03 | 8.18E−11 | hsa03050:Proteasome |
| | | | alpha type, 5 | | | | | | |
| 19 | PSMD11 | 208777_s_at | proteasome 26S | BC000437 | 17q11.2 | 4.7E−06 | 8.7E−06 | 3.50E−16 | hsa03050:Proteasome |
| | | | subunit, non-ATPase, | | | | | | |
| | | | 11 | | | | | | |
| 20 | PSMC3 | 201267_s_at | proteasome 26S | BC008713 | 11p11.2 | 4.7E−06 | 5.6E−04 | 1.46E−12 | hsa03050:Proteasome |
| | | | subunit, ATPase, 3 | | | | | | |
| 21 | PSMA4 | 203396_at | proteasome subunit, | BC005361 | 15q25.1 | 4.7E−06 | 6.3E−04 | 1.87E−12 | hsa03050:Proteasome |
| | | | alpha type, 4 | | | | | | |
| 22 | PSMC2 | 201068_s_at | proteasome 26S | BC002589 | 7q22.1- | 4.7E−06 | 4.8E−05 | 1.06E−14 | hsa03050:Proteasome |
| | | | subunit, ATPase, 2 | | q22.3 | | | | |
| 23 | PSMC1 | 204219_s_at | proteasome 26S | BC000512 | 14q32.11 | 4.7E−06 | 2.2E−03 | 2.27E−11 | hsa03050:Proteasome |
| | | | subunit, ATPase, 1 | | | | | | |
| 24 | PSMA3 | 201532_at | proteasome subunit, | BC005265 | 14q23 | 4.7E−06 | 2.4E−03 | 2.64E−11 | hsa03050:Proteasome |
| | | | alpha type, 3 | | | | | | |
| 25 | POMP | 217769_s_at | proteasome maturation | BC003390 | 13q12.3 | 4.7E−06 | 4.0E−03 | 7.57E−11 | hsa03050:Proteasome |
| | | | protein | | | | | | |
| 26 | PSME3 | 209853_s_at | proteasome activator | BC008020 | 17q21 | 1.1E−05 | 2.7E−04 | 7.91E−13 | hsa03050:Proteasome |
| | | | subunit 3 | | | | | | |
| 27 | PSME4 | 212219_at | proteasome activator | BC112169 | 2p16.2 | 4.7E−06 | 1.6E−04 | 1.22E−13 | hsa03050:Proteasome |
| | | | subunit 4 | | | | | | |
| 28 | E2F2 | 228361_at | E2F transcription factor 2 | BC053676 | 1p36 | 1.2E−03 | 2.9E−03 | 1.03E−08 | hsa04110 |
| 29 | YWHAZ | 200639_s_at | tyrosine 3- | BC003623 | 8q23.1 | 4.7E−06 | 2.4E−03 | 2.58E−11 | hsa04110 |
| | | | monooxygenase | | | | | | |
| | | | activation protein | | | | | | |
| 30 | DBF4 | 204244_s_at | DBF4 homolog | BC047693 | 7q21.3 | 4.7E−06 | 1.4E−04 | 9.33E−14 | hsa04110 |
| 31 | TTK | 204822_at | TTK protein kinase | BC032858 | 6q13-q21 | 4.7E−06 | 8.7E−06 | 3.50E−16 | hsa04110 |
| 32 | PKMYT1 | 204267_x_at | protein kinase, | BC121162 | 16p13.3 | 4.7E−06 | 1.6E−04 | 1.22E−13 | hsa04110 |
| | | | membrane associated | | | | | | |
| | | | tyrosine/threonine 1 | | | | | | |
| 33 | CHEK1 | 205394_at | CHK1 checkpoint | BC004202 | 11q24.2 | 4.7E−06 | 8.7E−06 | 3.50E−16 | hsa04110 |
| | | | homolog ( [symbol] ) | | | | | | |
| 34 | ANAPC11 | 226414_s_at | anaphase promoting | BC000607 | 17q25.3 | 4.7E−06 | 4.2E−04 | 8.37E−13 | hsa04110 |
| | | | complex subunit 11 | | | | | | |
| 35 | PTTG1 | 203554_x_at | pituitary tumor- | BC128193 | 5q35.1 | 4.7E−06 | 8.7E−06 | 3.50E−16 | hsa04110 |
| | | | transforming 1 | | | | | | |
| 36 | RBX1 | 218117_at | ring-box 1, E3 ubiquitin | BC001466 | 22q13.2 | 4.7E−06 | 3.4E−03 | 5.32E−11 | hsa04110 |
| | | | protein ligase | | | | | | |
| 37 | CCNE2 | 205034_at | cyclin E2 | BCO20729 | 8q22.1 | 4.7E−06 | 8.4E−05 | 3.27E−14 | hsa04110 |
| 38 | CCNE1 | 213523_at | cyclin E1 | BC035498 | 19q12 | 6.6E−05 | 4.3E−05 | 1.19E−13 | hsa04110 |
| 39 | CDC45 | 204126_s_at | cell division cycle 45 | BC010022 | 22q11.21 | 4.7E−06 | 8.7E−06 | 3.50E−16 | hsa04110 |
| | | | homolog ( [symbol] ) | | | | | | |
| 40 | MCM7 | 208795_s_at | minichromosome | BC013375 | 7q21.3- | 1.0E−03 | 1.6E−05 | 2.60E−13 | hsa04110 |
| | | | maintenance complex | | q22.1 | | | | |
| | | | component 7 | | | | | | |
| 41 | RAD21 | 200608_s_at | RAD21 homolog | BC050381 | 8q24 | 1.1E−05 | 2.7E−04 | 8.34E−13 | hsa04110 |
| | | | ( [symbol] ) | | | | | | |
| 42 | BUB1 | 209642_at | budding uninhibited by | BC028201 | 2q14 | 4.7E−06 | 8.7E−06 | 3.50E−16 | hsa04110 |
| | | | benzimidazoles 1 | | | | | | |
| | | | homolog | | | | | | |
| 43 | CCNA2 | 203418_at | cyclin A2 | BC104783 | 4q25-q31 | 4.7E−06 | 8.7E−06 | 3.50E−16 | hsa04110 |
| 44 | TFDP1 | 212330_at | transcription factor Dp-1 | BC011685 | 13q34 | 8.2E−04 | 1.2E−03 | 1.09E−09 | hsa04110 |
| 45 | CDC7 | 204510_at | cell division cycle 7 | BC111044 | 1p22 | 1.2E−04 | 6.1E−03 | 4.43E−09 | hsa04110 |
| | | | homolog ( [symbol] ) | | | | | | |
| 46 | CDC6 | 203967_at | cell division cycle 6 | BC025232 | 17q21.3 | 2.2E−05 | 8.7E−06 | 1.67E−15 | hsa04110 |
| | | | homolog ( [symbol] ) | | | | | | |
| 47 | CDK1 | 203214_x_at | cyclin-dependent | BC014563 | 10q21.1 | 4.7E−06 | 8.7E−06 | 3.50E−16 | hsa04114 |
| | | | kinase 1 | | | | | | |
| 48 | SKP2 | 203625_x_at | S-phase kinase- | BC001441 | 5p13 | 1.8E−04 | 1.3E−04 | 3.07E−12 | hsa04110 |
| | | | associated protein 2 | | | | | | |
| | | | (p45) | | | | | | |
| 49 | ESPL1 | 38158_at | extra spindle pole | na | 12q | 4.7E−06 | 8.7E−06 | 3.50E−16 | hsa04110 |
| | | | bodies homolog 1 | | | | | | |
| | | | (S. cerevisiae) | | | | | | |
| 50 | MCM2 | 202107_s_at | minichromosome | BC014272 | 3q21 | 4.7E−06 | 8.7E−06 | 3.50E−16 | hsa04110 |
| | | | maintenance complex | | | | | | |
| | | | component 2 | | | | | | |
| 51 | MCM3 | 201555_at | minichromosome | BC014272 | 6p12 | 4.7E−06 | 8.7E−06 | 3.50E−16 | hsa04110 |
| | | | maintenance complex | | | | | | |
| | | | component 3 | | | | | | |
| 52 | CDK4 | 202246_s_at | cyclin-dependent | BC003644 | 12q14 | 4.7E−06 | 3.7E−04 | 6.50E−13 | hsa04110 |
| | | | kinase 4 | | | | | | |
| 53 | CDC25C | 205167_s_at | cell division cycle 25 | BC019089 | 5q31 | 4.7E−06 | 3.3E−04 | 4.95E−13 | hsa04110 |
| | | | homolog C (S. pombe) | | | | | | |
| 54 | MCM4 | 212141_at | minichromosome | BC031061 | 8q11.2 | 1.4E−05 | 8.7E−06 | 1.02E−15 | hsa04110 |
| | | | maintenance complex | | | | | | |
| | | | component 4 | | | | | | |
| 55 | MCM5 | 216237_s_at | minichromosome | BC003656 | 22q13.1 | 4.7E−06 | 8.7E−06 | 3.50E−16 | hsa04110 |
| | | | maintenance complex | | | | | | |
| | | | component 5 | | | | | | |
| 56 | CDC25A | 204695_at | cell division cycle 25 | BC007401 | 3p21 | 4.7E−06 | 8.7E−06 | 3.50E−16 | hsa04110 |
| | | | homolog A (S. pombe) | | | | | | |
| 57 | MCM6 | 201930_at | minichromosome | BC032374 | 2q21 | 4.7E−06 | 8.7E−06 | 3.50E−16 | hsa04110 |
| | | | maintenance complex | | | | | | |
| | | | component 6 | | | | | | |
| 58 | CCNB1 | 214710_s_at | cyclin B1 | BC006510 | 5q12 | 4.7E−06 | 8.7E−06 | 3.50E−16 | hsa04110 |
| 59 | MAD2L1 | 203362_s_at | MAD2 mitotic arrest | BC000356 | 4q27 | 4.7E−06 | 8.7E−06 | 3.50E−16 | hsa04110 |
| | | | deficient-like 1 (yeast) | | | | | | |
| 60 | CCNB2 | 202705_at | cyclin B2 | BC105086 | 15q22.2 | 4.7E−06 | 8.7E−06 | 3.50E−16 | hsa04110 |
| 61 | PLK1 | 202240_at | polo-like kinase 1 | BC002369 | 16p12.2 | 4.7E−06 | 8.7E−06 | 3.50E−16 | hsa04110 |
| 62 | YWHAQ | 200693_at | tyrosine 3- | BC056867 | 2p25.1 | 1.1E−05 | 5.1E−04 | 2.85E−12 | hsa04110 |
| | | | monooxygenase/ | | | | | | |
| | | | activation protein | | | | | | |
| 63 | PCNA | 201202_at | proliferating cell nuclear | BC000491 | 20pter-p12 | 4.7E−06 | 8.7E−06 | 3.50E−16 | hsa04110 |
| | | | antigen | | | | | | |
| 64 | BUB1B | 203755_at | budding uninhibited by | BC018739 | 15q15 | 4.7E−06 | 8.7E−06 | 3.50E−16 | hsa04110 |
| | | | benzimidazoles 1 | | | | | | |
| | | | homolog beta | | | | | | |
| 65 | ANAPC7 | 225554_s_at | anaphase promoting | BC148236 | 12q24.11 | 4.7E−06 | 9.2E−03 | 3.94E−10 | hsa04110 |
| | | | complex subunit 7 | | | | | | |
| 66 | SSBP1 | 202591_s_at | single-stranded DNA | BC000895 | 7q34 | 4.7E−06 | 4.7E−03 | 1.01E−10 | hsa03430 |
| | | | binding protein 1 | | | | | | |
| 67 | RNASEH1 | 218497_s_at | ribonuclease H1 | BC002973 | 2p25 | 4.7E−06 | 8.7E−06 | 3.50E−16 | hsa03030 |
| 68 | POLA2 | 204441_s_at | polymerase (DNA | BC001347 | 11q13.1 | 4.7E−06 | 8.2E−03 | 3.15E−10 | hsa03030 |
| | | | directed), alpha 2 | | | | | | |
| 69 | RNASEH2A | 203022_at | ribonuclease H2, | BC011748 | 19p13.2 | 4.7E−06 | 8.7E−06 | 3.50E−16 | hsa03030 |
| | | | subunit A | | | | | | |
| 70 | RFC5 | 203210_s_at | replication factor C | BC001866 | 12q24.23 | 5.3E−05 | 2.6E−04 | 3.48E−12 | hsa03430 |
| | | | (activator 1) 5, 36.5 kDa | | | | | | |
| 71 | PRIM1 | 205053_at | primase, DNA, | BC005266 | 12q13 | 1.4E−05 | 1.5E−04 | 3.04E−13 | hsa03030 |
| | | | polypeptide 1 (49 kDa) | | | | | | |
| 72 | RFC3 | 204127_at | replication factor C | BC000149 | 13q13.2 | 4.7E−06 | 1.5E−04 | 1.04E−13 | hsa03030 |
| | | | (activator 1) 3, 38 kDa | | | | | | |
| 73 | RFC4 | 204023_at | replication factor C | BC024022 | 3q27 | 4.7E−06 | 8.7E−06 | 3.50E−16 | hsa03030 |
| | | | (activator 1) 4, 37 kDa | | | | | | |
| 74 | RFC2 | 1053_at | replication factor C | BC002813 | 7q11.23 | 4.7E−06 | 7.9E−05 | 2.93E−14 | hsa03030 |
| | | | (activator 1) 2, 40 kDa | | | | | | |
| 75 | POLD1 | 203422_at | polymerase (DNA | BC008800 | 19q13.3 | 4.7E−06 | 1.9E−04 | 1.70E−13 | hsa03030 |
| | | | directed), delta 1, | | | | | | |
| | | | catalytic subunit 125 kDa | | | | | | |
| 76 | POLD2 | 201115_at | polymerase (DNA | BC000459 | 7p13 | 4.7E−06 | 4.5E−03 | 9.43E−11 | hsa03030 |
| | | | directed), delta 2, | | | | | | |
| | | | regulatory subunit | | | | | | |
| | | | 50 kDa | | | | | | |
| 77 | FEN1 | 204768_s_at | flap structure-specific | BC000323 | 11q12 | 4.7E−06 | 8.7E−06 | 3.50E−16 | hsa03030 |
| | | | endonuclease 1 | | | | | | |
| 78 | NCBP1 | 209520_s_at | nuclear cap binding | BC001450 | 9q34.1 | 6.0E−04 | 2.8E−03 | 4.61E−09 | hsa03040:Spliceosome |
| | | | protein subunit 1, | | | | | | |
| | | | 80 kDa | | | | | | |
| 79 | NHP2L1 | 201077_s_at | NHP2 non-histone | BC005358 | 22q13 | 4.7E−06 | 6.3E−04 | 1.82E−12 | hsa03040:Spliceosome |
| | | | chromosome protein 2- | | | | | | |
| | | | like 1 | | | | | | |
| 80 | PPIL1 | 222500_at | peptidylprolyl | BC003048 | 6p21.1 | 1.1E−05 | 1.1E−03 | 1.34E−11 | hsa03040:Spliceosome |
| | | | isomerase (cyclophilin)- | | | | | | |
| | | | like 1 | | | | | | |
| 81 | LSM7 | 204559_s_at | LSM7 homolog, U6 | BC018621 | 19p13.3 | 4.7E−06 | 1.1E−03 | 5.76E−12 | hsa03040:Spliceosome |
| | | | small nuclear RNA | | | | | | |
| | | | associated | | | | | | |
| 82 | SNRPD1 | 202690_s_at | small nuclear | BC001721 | 18q11.2 | 4.7E−06 | 8.7E−06 | 3.50E−16 | hsa03040:Spliceosome |
| | | | ribonucleoprotein D1 | | | | | | |
| | | | polypeptide 16 kDa | | | | | | |
| 83 | SNRPD2 | 200826_at | small nuclear | BC000486 | 19q13.2 | 4.7E−06 | 5.7E−04 | 1.51E−12 | hsa03040:Spliceosome |
| | | | ribonucleoprotein D2 | | | | | | |
| | | | polypeptide 16.5 kDa | | | | | | |
| 84 | SF3B5 | 221263_s_at | splicing factor 3b, | BC000198 | 6q24.2 | 4.7E−06 | 2.7E−04 | 3.49E−13 | hsa03040:Spliceosome |
| | | | subunit 5, 10 kDa | | | | | | |
| 85 | SF3B3 | 200687_s_at | splicing factor 3b, | BC003146 | 16q22.1 | 4.7E−06 | 8.7E−06 | 3.50E−16 | hsa03040:Spliceosome |
| | | | subunit 3, 130 kDa | | | | | | |
| 86 | HNRNPA3 | 211930_at | heterogeneous nuclear | BC064494 | 2q31.2 | 4.7E−06 | 7.3E−03 | 2.46E−10 | hsa03040:Spliceosome |
| | | | ribonucleoprotein A3 | | | | | | |
| 87 | HNRNPK | 200775_s_at | heterogeneous nuclear | BC000355 | 9q21.32- | 1.0E−02 | 1.6E−03 | 2.40E−08 | hsa03040:Spliceosome |
| | | | ribonucleoprotein K | | q21.33 | | | | |
| 88 | RBM8A | 222443_s_at | RNA binding motif | BC017088 | 1q12 | 3.5E−04 | 8.7E−06 | 2.59E−14 | hsa03040:Spliceosome |
| | | | protein 8A | | | | | | |
| 89 | USP39 | 217829_s_at | ubiquitin specific | BC067273 | 2p11.2 | 1.4E−05 | 1.4E−04 | 2.73E−13 | hsa03040:Spliceosome |
| | | | peptidase 39 | | | | | | |
| 90 | LSM4 | 202737_s_at | LSM4 homolog, U6 | BC000387 | 19p13.11 | 4.7E−06 | 8.7E−06 | 3.50E−16 | hsa03040:Spliceosome |
| | | | small nuclear RNA | | | | | | |
| | | | associated | | | | | | |
| 91 | LSM3 | 202209_at | LSM3 homolog, U6 | BC007055 | 3p25.1 | 8.1E−04 | 3.7E−03 | 1.13E−08 | hsa03040:Spliceosome |
| | | | small nuclear RNA | | | | | | |
| | | | associated | | | | | | |
| 92 | SNRPA1 | 215722_s_at | small nuclear | BC022816 | 15q26.3 | 2.8E−05 | 8.7E−06 | 2.08E−15 | hsa03040:Spliceosome |
| | | | ribonucleoprotein | | | | | | |
| | | | polypeptide A′ | | | | | | |
| 93 | EFTUD2 | 222398_s_at | elongation factor Tu | BC002360 | 17q21.31 | 4.7E−06 | 5.4E−03 | 1.36E−10 | hsa03040:Spliceosome |
| | | | GTP binding domain | | | | | | |
| | | | containing 2 | | | | | | |
| 94 | PRPF18 | 221547_at | PRP18 pre-mRNA | BC000794 | 10p13 | 2.2E−03 | 6.7E−05 | 9.91E−12 | hsa03040:Spliceosome |
| | | | processing factor 18 | | | | | | |
| | | | homolog | | | | | | |
| 95 | EIF4A3 | 201303_at | eukaryotic translation | BC004386 | 17q25.3 | 4.7E−06 | 8.7E−06 | 3.50E−16 | hsa03040:Spliceosome |
| | | | initiation factor 4A3 | | | | | | |
| 96 | SNRPB | 213175_s_at | small nuclear | BC080516 | 20p13 | 4.7E−06 | 1.5E−04 | 9.78E−14 | hsa03040:Spliceosome |
| | | | ribonucleoprotein | | | | | | |
| | | | polypeptides B and B1 | | | | | | |
| 97 | SNRPA | 201770_at | small nuclear | BC000405 | 19q13.1 | 1.9E−04 | 4.5E−03 | 3.91E−09 | hsa03040:Spliceosome |
| | | | ribonucleoprotein | | | | | | |
| | | | polypeptide A | | | | | | |
| 98 | SNRPC | 201342_at | small nuclear | BC121082 | 6p21.31 | 4.7E−06 | 8.7E−06 | 3.50E−16 | hsa03040:Spliceosome |
| | | | ribonucleoprotein | | | | | | |
| | | | polypeptide C | | | | | | |
| 99 | SNRNP27 | 212438_at | small nuclear | BC017890 | 2p13.3 | 1.1E−05 | 4.6E−03 | 2.39E−10 | hsa03040:Spliceosome |
| | | | ribonucleoprotein | | | | | | |
| | | | 27 kDa (U4/U6.U5) | | | | | | |
| 100 | PUF60 | 209899_s_at | poly-U binding splicing | BC008875 | 8q24.3 | 1.1E−05 | 8.7E−06 | 8.34E−16 | hsa03040:Spliceosome |
| | | | factor 60 kDa | | | | | | |
| 101 | SNRPG | 205644_s_at | small nuclear | BC000070 | 2p13.3 | 4.7E−06 | 8.7E−06 | 3.50E−26 | hsa03040:Spliceosome |
| | | | ribonucleoprotein | | | | | | |
| | | | polypeptide G | | | | | | |
| 102 | RBM17 | 224781_s_at | RNA binding motif | BC007871 | 10p15.1 | 1.1E−05 | 8.7E−06 | 8.34E−16 | hsa03040:Spliceosome |
| | | | protein 17 | | | | | | |
| 103 | RFC5 | 203210_s_at | replication factor C | BC001866 | 12q24.23 | 5.3E−05 | 2.6E−04 | 3.48E−12 | hsa03430 |
| | | | (activator 1) 5, 36.5 kDa | | | | | | |
| 104 | EXO1 | 204603_at | exonuclease 1 | BC007491 | 1q42-q43 | 6.4E−05 | 8.7E−06 | 4.83E−15 | hsa03430 |
| 105 | MSH6 | 202911_at | mutS homolog 6 (E. coli) | BC004246 | 2p16 | 4.7E−06 | 8.7E−06 | 3.50E−16 | hsa03430 |
| 106 | SSBP1 | 202591_s_at | single-stranded DNA | BC000895 | 7q34 | 4.7E−06 | 4.7E−03 | 1.01E−10 | hsa03430 |
| | | | binding protein 1 | | | | | | |
| 107 | MSH2 | 209421_at | mutS homolog 2, colon | BC021566 | 2p21 | 4.9E−04 | 9.4E−05 | 4.35E−12 | hsa03430 |
| | | | cancer, nonpolyposis | | | | | | |
| | | | type 1 (E. coli) | | | | | | |
| 108 | POLR2H | 209302_at | polymerase (RNA) II | BC000739 | 3q28 | 4.7E−06 | 8.3E−03 | 3.21E−10 | hsa00240 |
| | | | (DNA directed) | | | | | | |
| | | | polypeptide H | | | | | | |
| 109 | POLR2F | 209511_at | polymerase (RNA) II | BC003582 | 22q13.1 | 1.4E−05 | 1.2E−04 | 2.09E−13 | hsa00240 |
| | | | (DNA directed) | | | | | | |
| | | | polypeptide F | | | | | | |
| 110 | POLR2K | 202635_s_at | polymerase (RNA) II | BC000806 | 8q22.2 | 4.7E−06 | 6.3E−04 | 1.87E−12 | hsa00240 |
| | | | (DNA directed) | | | | | | |
| | | | polypeptide K, 7.0 kDa | | | | | | |
| 111 | POLR21 | 212782_x_at | polymerase (RNA) II | BC065711 | 7q22.1 | 4.7E−06 | 1.3E−04 | 7.93E−14 | hsa00240 |
| | | | (DNA directed) | | | | | | |
| | | | polypeptide J, 13.3 kDa | | | | | | |
| 112 | PNPT1 | 225291_at | polyribonucleotide | BC053660 | 2p15 | 4.7E−06 | 8.7E−06 | 3.50E−16 | hsa00240 |
| | | | nucleotidyltransferase 1 | | | | | | |
| 113 | DTYMK | 203270_at | deoxythymidylate | BC147010 | 2q37.3 | 4.7E−06 | 1.7E−03 | 1.41E−11 | hsa00240 |
| | | | kinase (thymidylate | | | | | | |
| | | | kinase) | | | | | | |
| 114 | POLR1C | 209317_at | polymerase (RNA) I | BC008863 | 6p21.1 | 1.7E−03 | 8.7E−06 | 1.26E−13 | hsa00240 |
| | | | polypeptide C, 30 kDa | | | | | | |
| 115 | ZNRD1 | 228009_x_at | zinc ribbon domain | BC010898 | 6p21.3 | 1.1E−05 | 1.2E−03 | 1.69E−11 | hsa00240 |
| | | | containing 1 | | | | | | |
| 116 | CAD | 202715_at | carbamoyl-phosphate | BC065510 | 2p22-p21 | 6.3E−04 | 5.3E−03 | 1.75E−08 | hsa00240 |
| | | | synthetase 2 | | | | | | |
| 117 | POLR1B | 223403_s_at | polymerase (RNA) I | BC110833 | 2q13 | 4.7E−06 | 1.5E−04 | 1.04E−13 | hsa00240 |
| | | | polypeptide B, 128 kDa | | | | | | |
| 118 | PNP | 201695_s_at | purine nucleoside | BC106074 | 14q13.1 | 2.6E−04 | 2.3E−03 | 1.33E−09 | hsa00240 |
| | | | phosphorylase | | | | | | |
| 119 | TK1 | 202338_at | thymidine kinase 1, | BC006484 | 17q23.2- | 4.7E−06 | 8.7E−06 | 3.50E−16 | hsa00240 |
| | | | soluble | | q25.3 | | | | |
| 120 | TYMS | 202589_at | thymidylate synthetase | BC002567 | 18p11.32 | 4.7E−06 | 8.7E−06 | 3.50E−16 | hsa00240 |
| 121 | UMPS | 202706_s_at | uridine monophosphate | BC000364 | 3q13 | 4.7E−06 | 6.7E−05 | 2.06E−14 | hsa00240 |
| | | | synthetase | | | | | | |
| 122 | NME1 | 201577_at | non-metastatic cells 1, | BC002476 | 17q21.3 | 4.7E−06 | 8.7E−06 | 3.50E−16 | hsa00240 |
| | | | protein (NM23A) | | | | | | |
| | | | expressed in | | | | | | |
| 123 | UCK2 | 209825_s_at | uridine-cytidine kinase 2 | BC002906 | 1q23 | 4.7E−06 | 8.7E−06 | 3.50E−16 | hsa00240 |
| 124 | IARS | 204744_s_at | isoleucyl-tRNA | BC065552 | 9q21 | 2.9E−04 | 4.9E−04 | 6.86E−11 | hsa00970 |
| | | | synthetase | | | | | | |
| 125 | TARS | 201263_at | threonyl-tRNA | BC000517 | 5p13.2 | 4.7E−06 | 5.9E−03 | 1.59E−10 | hsa00970 |
| | | | synthetase | | | | | | |
| 126 | YARS | 212048_s_at | tyrosyl-tRNA synthetase | BC001933 | 1p35.1 | 4.7E−06 | 3.0E−05 | 4.21E−15 | hsa00970 |
| 127 | CARS | 212971_at | cysteinyl-tRNA | BC002880 | 11p15.5 | 4.7E−06 | 6.7E−03 | 2.11E−10 | hsa00970 |
| | | | synthetase | | | | | | |
| 128 | AARS | 201000_at | alanyl-tRNA synthetase | BC011451 | 16q22 | 4.7E−06 | 8.7E−06 | 3.50E−16 | hsa00970 |
| 129 | GARS | 208693_s_at | glycyl-tRNA synthetase | BC007755 | 7p15 | 4.7E−06 | 8.7E−06 | 3.50E−16 | hsa00970 |
| 130 | FARSB | 223035_s_at | phenylalanyl-tRNA | BC017783 | 2q36.1 | 4.7E−06 | 3.6E−04 | 6.13E−13 | hsa00970 |
| | | | synthetase, beta subunit | | | | | | |
| 131 | EPRS | 200843_s_at | glutamyl-prolyl-tRNA | BC126275 | 1q41 | 4.7E−06 | 8.7E−06 | 3.50E−16 | hsa00970 |
| | | | synthetase | | | | | | |
| 132 | DARS2 | 218365_s_at | aspartyl-tRNA | BC045173 | 1q25.1 | 1.6E−04 | 5.6E−04 | 4.93E−11 | hsa00970 |
| | | | synthetase 2, | | | | | | |
| | | | mitochondrial | | | | | | |
| 133 | VARS | 201797_s_at | valyl-tRNA synthetase | BC012808 | 6p21.3 | 4.7E−06 | 5.8E−03 | 1.55E−10 | hsa00970 |
| 134 | KARS | 200079_s_at | lysyl-tRNA synthetase | BC004132 | 16q23.1 | 4.7E−06 | 8.7E−06 | 3.50E−16 | hsa00970 |
| 135 | MARS | 201475_x_at | methionyl-tRNA | BC002384 | 12q13.2 | 4.7E−06 | 5.4E−04 | 1.36E−12 | hsa00970 |
| | | | synthetase | | | | | | |
| 136 | HPRT1 | 202854_at | hypoxanthine | BC000578 | Xq26.1 | 4.4E−04 | 8.7E−06 | 3.33E−14 | hsa00230 |
| | | | phosphoribosyl- | | | | | | |
| | | | transferase 1 | | | | | | |
| 137 | PPAT | 209434_s_at | phosphoribosyl | BC004200 | 4q12 | 4.7E−06 | 6.8E−03 | 2.16E−10 | hsa00230 |
| | | | pyrophosphate | | | | | | |
| | | | amidotransferase | | | | | | |
| 138 | ATIC | 208758_at | 5-aminoimidazole-4- | BC008879 | 2q35 | 4.7E−06 | 2.3E−03 | 2.36E−11 | hsa00230 |
| | | | carboxamide | | | | | | |
| | | | ribonucleotide | | | | | | |
| | | | formyltransferase | | | | | | |
| 139 | NUDT5 | 223100_s_at | nudix (nucleoside | BC000025 | 10p14 | 4.7E−06 | 8.7E−06 | 3.50E−16 | hsa00230 |
| | | | diphosphate linked | | | | | | |
| | | | moiety X)-type motif 5 | | | | | | |
| 140 | AK2 | 208967_s_at | adenylate kinase 2 | BC090040 | 1p34 | 4.7E−06 | 9.5E−04 | 4.20E−12 | hsa00230 |
| 141 | DGUOK | 209549_s_at | deoxyguanosine kinase | BC015757 | 2p13 | 4.7E−06 | 1.4E−04 | 9.33E−14 | hsa00230 |
| 142 | GMPS | 214431_at | guanine monphosphate | BC012178 | 3q24 | 4.7E−06 | 8.7E−06 | 3.50E−16 | hsa00230 |
| | | | synthetase | | | | | | |
| 143 | APRT | 203219_s_at | adenine phospho- | BC107151 | 16q24 | 4.7E−06 | 8.7E−06 | 3.50E−16 | hsa00230 |
| | | | ribosyltransferase | | | | | | |
| 144 | GART | 212378_at | phosphoribosylglycin- | BC107712 | 21q22.1 | 2.2E−05 | 8.7E−06 | 1.67E−15 | hsa00230 |
| | | | amide formyltransferase | | | | | | |
| 145 | ADSL | 202144_s_at | adenylosuccinate lyase | BC000253 | 22q13.1 | 7.5E−04 | 1.7E−03 | 2.05E−09 | hsa00230 |
| 146 | PAICS | 201014_s_at | phosphoribosylamino- | BC019255 | 4q12 | 4.7E−06 | 8.7E−06 | 3.50E−16 | hsa00230 |
| | | | imidazole carboxylase | | | | | | |
| 147 | CDK1 | 203214_x_at | cyclin-dependent | BC014563 | 10q21.1 | 4.7E−06 | 8.7E−06 | 3.50E−16 | hsa04114 |
| | | | kinase 1 | | | | | | |
| 148 | AURKA | 208079_s_at | aurora kinase A | BC002499 | 20q13 | 4.7E−06 | 8.7E−06 | 3.50E−16 | hsa04114 |
| 149 | FBXO5 | 234863_x_at | F-box protein 5 | BC018905 | 6q25.2 | 1.4E−05 | 8.7E−06 | 1.02E−15 | hsa04114 |
| 150 | MAD2L2 | 223234_at | MAD2 mitotic arrest | BC015244 | 1p36 | 6.3E−05 | 4.0E−04 | 1.03E−11 | hsa04110 |
| | | | deficient-like 2 (yeast) | | | | | | |

### REFERENCES

1. Ferlay J, Shin H R, Bray F, Forman D, Mathers C, et al. (2010) Estimates of worldwide burden of cancer in 2008: GLOBOCAN 2008. Int J Cancer 127: 2893-2917.
2. Paap E, Holland R, den Heeten G J, van Schoor G, Botterweck A A, et al. (2010) A remarkable reduction of breast cancer deaths in screened versus unscreened women: a case-referent study. Cancer Causes Control 21: 1569-1573.
3. Group EBCTC (2005) Effects of chemotherapy and hormonal therapy for early breast cancer on recurrence and 15-year survival: an overview of the randomised trials. Lancet 365: 1687-1717.
4. Andre F, Michiels S, Dessen P, Scott V, Suciu V, et al. (2009) Exonic expression profiling of breast cancer and benign lesions: a retrospective analysis. Lancet Oncol 10: 381-390.
5. Andre F, Pusztai L (2006) Molecular classification of breast cancer: implications for selection of adjuvant chemotherapy. Nat Clin Pract Oncol 3: 621-632.
6. Campbell J D, Ramsey S D (2009) The costs of treating breast cancer in the U S: a synthesis of published evidence. Pharmacoeconomics 27: 199-209.
7. Gentry C (2002) Improving Quality of Care for Californians with Breast Cancer California Healthcare Foundation http://www.chcf.org/˜/media/MEDIA%20LIBRARY%20Files/PDF/I/PDF%20Improving QualityBreastCancer.pdf.
8. Elston C W, Ellis 10 (1991) Pathological prognostic factors in breast cancer. I. The value of histological grade in breast cancer: experience from a large study with long-term follow-up. Histopathology 19: 403-410.
9. Balslev I, Axelsson C K, Zedeler K, Rasmussen B B, Carstensen B, et al. (1994) The Nottingham Prognostic Index applied to 9,149 patients from the studies of the Danish Breast Cancer Cooperative Group (DBCG). Breast Cancer Res Treat 32: 281-290.
10. Singletary S E, Allred C, Ashley P, Bassett L W, Berry D, et al. (2002) Revision of the American Joint Committee on Cancer staging system for breast cancer. J Clin Oncol 20: 3628-3636.
11. Sotiriou C, Wirapati P, Loi S, Harris A, Fox S, et al. (2006) Gene expression profiling in breast cancer: understanding the molecular basis of histologic grade to improve prognosis. J Natl Cancer Inst 98: 262-272.
12. Calza S, Hall P, Auer G, Bjohle J, Klaar S, et al. (2006) Intrinsic molecular signature of breast cancer in a population-based cohort of 412 patients. Breast Cancer Res 8: R34.
13. Sotiriou C, Neo S Y, McShane L M, Korn E L, Long P M, et al. (2003) Breast cancer classification and prognosis based on gene expression profiles from a population-based study. Proc Natl Acad Sci USA 100: 10393-10398.
14. Finetti P, Cervera N, Charafe-Jauffret E, Chabannon C, Charpin C, et al. (2008) Sixteen-kinase gene expression identifies luminal breast cancers with poor prognosis. Cancer Res 68: 767-776.
15. Sabatier R, Finetti P, Mamessier E, Raynaud S, Cervera N, et al. (2011) Kinome expression profiling and prognosis of basal breast cancers. Mol Cancer 10: 86.
16. Gordon G J, Jensen R V, Hsiao L L, Gullans S R, Blumenstock J E, et al. (2002) Translation of microarray data into clinically relevant cancer diagnostic tests using gene expression ratios in lung cancer and mesothelioma. Cancer Res 62: 4963-4967.
17. Ma X J, Hilsenbeck S G, Wang W, Ding L, Sgroi D C, et al. (2006) The HOXB13:IL17B R expression index is a prognostic factor in early-stage breast cancer. J Clin Oncol 24: 4611-4619.
18. Fan C, Oh D S, Wessels L, Weigelt B, Nuyten D S, et al. (2006) Concordance among gene-expression-based predictors for breast cancer. N Engl J Med 355: 560-569.
19. van't Veer L J, Dal H, van de Vijver M J, He Y D, Hart A A, et al. (2002) Gene expression profiling predicts clinical outcome of breast cancer. Nature 415: 530-536.
20. Bertucci F, Finetti P, Cervera N, Chafafe-Jauffret E, Buttarelli M, et al. (2009) How different are luminal A and basal breast cancers? Int J Cancer 124: 1338-1348.
21. (2005) Effects of chemotherapy and hormonal therapy for early breast cancer on recurrence and 15-year survival: an overview of the randomised trials. Lancet 365: 1687-1717.
22. Ivshina A V, George J, Senko O, Mow B, Putti T C, et al. (2006) Genetic reclassification of histologic grade delineates new clinical subtypes of breast cancer. Cancer Res 66: 10292-10301.
23. Katayama S, Tomaru Y, Kasukawa T, Waki K, Nakanishi M, et al. (2005) Antisense transcription in the mammalian transcriptome. Science 309: 1564-1566.
24. Faghihi M A, Modarresi F, Khalil A M, Wood D E, Sahagan B G, et al. (2008) Expression of a noncoding RNA is elevated in Alzheimer's disease and drives rapid feed-forward regulation of beta-secretase. Nat Med 14: 723-730.
25. Hastings M L, Milcarek C, Martincic K, Peterson M L, Munroe S H (1997) Expression of the thyroid hormone receptor gene, erbAalpha, in B lymphocytes: alternative mRNA processing is independent of differentiation but correlates with antisense RNA levels. Nucleic Acids Res 25: 4296-4300.
26. Morris K V, Santoso S, Turner A M, Pastori C, Hawkins P G (2008) Bidirectional transcription directs both transcriptional gene activation and suppression in human cells. PLoS Genet 4: e1000258.
27. Morrissy A S, Griffith M, Marra M A (2011) Extensive relationship between antisense transcription and alternative splicing in the human genome. Genome Res 21: 1203-1212.
28. Xu Z, Wei W, Gagneur J, Clauder-Munster S, Smolik M, et al. Antisense expression increases gene expression variability and locus interdependency. Mol Syst Biol 7: 468.
29. Grinchuk O V, Jenjaroenpun P, Orlov Y L, Zhou J, Kuznetsov V A (2010) Integrative analysis of the human cis-antisense gene pairs, miRNAs and their transcription regulation patterns. Nucleic Acids Res 38: 534-547.
30. Lapidot M, Pilpel Y (2006) Genome-wide natural antisense transcription: coupling its regulation to its different regulatory mechanisms. EMBO Rep 7: 1216-1222.
31. Morrissy A S (2010) Bioinformatic analysis of cis-encoded antisense transcription. [PhD Thesis].
32. Kohno K, Chiba M, Murata S, Pak S, Nagai K, et al. (2010) Identification of natural antisense transcripts involved in human colorectal cancer development. Int J Oncol 37: 1425-1432.
33. Maruyama R, Shipitsin M, Choudhury S, Wu Z, Protopopov A, et al. (2010) Breast Cancer Special Feature: Altered antisense-to-sense transcript ratios in breast cancer. Proc Natl Acad Sci USA.
34. Nordlund J, Kiialainen A, Karlberg O, Berglund E C, Goransson-Kultima H, et al. (2011) Digital gene expression profiling of primary acute lymphoblastic leukemia cells. Leukemia: 1-10.
35. Smyth G K (2004) Linear models and empirical bayes methods for assessing differential expression in microarray experiments. Stat Appl Genet Mol Biol 3: Article3.
36. Bullard J H, Purdom E, Hansen K D, Dudoit S (2010) Evaluation of statistical methods for normalization and differential expression in mRNA-Seq experiments. BMC Bioinformatics 11: 94.
37. Motakis E, Ivshina A V, Kuznetsov V A (2009) Data-driven approach to predict survival of cancer patients: estimation of microarray genes' prediction significance by Cox proportional hazard regression model. IEEE Eng Med Biol Mag 28: 58-66.
38. Ma X J, Wang Z, Ryan P D, Isakoff S J, Barmettler A, et al. (2004) A two-gene expression ratio predicts clinical outcome in breast cancer patients treated with tamoxifen. Cancer Cell 5: 607-616.
39. Paik S, Shak S, Tang G, Kim C, Baker J, et al. (2004) A multigene assay to predict recurrence of tamoxifen-treated, node-negative breast cancer. N Engl J Med 351: 2817-2826.
40. Jones C, Ford E, Gillett C, Ryder K, Merrett S, et al. (2004) Molecular cytogenetic identification of subgroups of grade III invasive ductal breast carcinomas with different ethical outcomes. Clin Cancer Res.10: 5988-5997.
41. Teschendorff A E, Caldas C (2008) A robust classifier of high predictive value to identify good prognosis patients in E R-negative breast cancer. Breast Cancer Res 10: R73.
42. Hallett R, Dvorkin-Gheva A, Bane A, Hassell J A (2012) A gene signature for predicting outcome in patients with basal-like breast cancer. Scientific Reports 2:227.
43. de Sousa EMF, Colak S, Buikhuisen J, Koster J, Cameron K, et al. (2011) Methylation of cancer-stem-cell-associated Wnt target genes predicts poor prognosis in colorectal cancer patients. Cell Stem Cell 9: 476-485.
44. Hou J, Aerts J, den Hamer B, van ljcken W, den Bakker M, et al. (2010) Gene expression-based classification of non-small cell lung carcinomas and survival prediction. PLoS One 5: e10312.
45. Corrionero A, Minana B, Valcarcel J (2011) Reduced fidelity of branch point recognition and alternative splicing induced by the anti-tumor drug spliceostatin A. Genes Dev 25: 445-459.
46. Fan L, Lagisetti C, Edwards C C, Webb T R, Potter P M (2011) Sudemycins, novel small molecule analogues of FR901464, induce alternative gene splicing. ACS Chem Biol 6: 582-589.
47. Webb T R, Joyner A S, Potter P M (2012) The development and application of small molecule modulators of SF3b as therapeutic agents for cancer. Drug Discov Today.
48. Bonnal S, Vigevani L, Valcarcel J (2012) The spliceosome as a target of novel antitumour drugs. Nat Rev Drug Discov 11: 847-859.
49. Roybal G A, Jurica M S (2010) Spliceostatin A inhibits spliceosome assembly subsequent to prespliceosome formation. Nucleic Acids Res 38: 6664-6672.
50. O'Brien K, Matlin A J, Lowell A M, Moore M J (2008) The biflavonoid isoginkgetin is a general inhibitor of Pre-mRNA splicing. J Biol Chem 283: 33147-33154.
51. Kelley J R, Brown J M, Frasier M M, Baron P L, Schweinfest C W, et al. (2000) The cancer-associated Sm-like oncogene: a novel target for the gene therapy of pancreatic cancer. Surgery 128: 353-360.
52. Kelley J R, Fraser M M, Hubbard J M, Watson D K, Cole D J (2003) CaSm antisense gene therapy: a novel approach for the treatment of pancreatic cancer. Anticancer Res 23: 2007-2013.
53. Albert B J, Sivaramakrishnan A, Naka T, Czaicki N L, Koide K (2007) Total syntheses, fragmentation studies, and antitumor/antiproliferative activities of FR901464 and its low picomolar analogue. J Am Chem Soc 129: 2648-2659.
54. Sampath J, Long P R, Shepard R L, Xia X, Devanarayan V, et al. (2003) Human SPF45, a splicing factor, has limited expression in normal tissues, is overexpressed in many tumors, and can confer a multidrug-resistant phenotype to cells. Am J Pathol 163: 1781-1790.
55. Goetz M P, Suman V J, Ingle J N, Nibbe A M, Visscher D W, et al. (2006) A two-gene expression ratio of homeobox 13 and interleukin-17B receptor for prediction of recurrence and survival in women receiving adjuvant tamoxifen. Clin Cancer Res 12: 2080-2087.
56. Spitzer T L, Rojas A, Zelenko Z, Aghajanova L, Erikson D W, et al. (2012) Perivascular human endometrial mesenchymal stem cells express pathways relevant to self-renewal, lineage specification, and functional phenotype. Biol Reprod 86: 58.
57. Livak K J, Schmittgen T D (2001) Analysis of relative gene expression data using real-time quantitative PCR and the 2(-Delta Delta C(T)) Method. Methods 25: 402-408.
58. Leek J T, Monsen E, Dabney A R, Storey J D (2006) EDGE: extraction and analysis of differential gene expression. Bioinformatics 22: 507-508.
59. Wahl M C, Will C L, Luhrmann R (2009) The spliceosome: design principles of a dynamic RNP machine. Cell 136: 701-718.
60. Hideshima T, Richardson P, Chauhan D, Palombella V J, Elliott P J, et al. (2001) The proteasome inhibitor PS-341 inhibits growth, induces apoptosis, and overcomes drug resistance in human multiple myeloma cells. Cancer Res 61: 3071-3076.
61. D'Arcy P, Brnjic S, Olofsson M H, Fryknas M, Lindsten K, et al. (2011) Inhibition of proteasome deubiquitinating activity as a new cancer therapy. Nat Med 17: 1636-1640.
62. Quidville V, Alsafadi S, Goubar A, Commo F, Scott V, et al. (2013) Targeting the deregulated spliceosome core machinery in cancer cells triggers mTOR blockade and autophagy. Cancer Res 73: 2247-2258.
63. Rossi D, Bruscaggin A, Spina V, Rasi S, Khiabanian H, et al. (2011) Mutations of the SF3B1 splicing factor in chronic lymphocytic leukemia: association with progression and fludarabine-refractoriness. Blood 118: 6904-6908.
64. Albert B J, McPherson P A, O'Brien K, Czaicki N L, Destefino V, et al. (2009) Meayamycin inhibits pre-messenger RNA splicing and exhibits picomolar activity against multidrug-resistant cells. Mol Cancer Ther 8: 2308-2318.
65. Kaida D, Motoyoshi H, Tashiro E, Nojima T, Hagiwara M, et al. (2007) Spliceostatin A targets SF3b and inhibits both splicing and nuclear retention of pre-mRNA. Nat Chem Biol 3: 576-583.
66. Hasegawa M, Miura T, Kuzuya K, Inoue A, Won Ki S, et al. (2011) Identification of SAP155 as the target of GEX1A (Herboxidiene), an antitumor natural product. ACS Chem Biol 6: 229-233.
67. Kotake Y, Sagane K, Owa T, Mimori-Kiyosue Y, Shimizu H, et al. (2007) Splicing factor SF3b as a target of the antitumor natural product pladienolide. Nat Chem Biol 3: 570-575.
68. Tsimberidou A M, Vaklavas C, Wen S, Hong D, Wheler J, et al. (2009) Phase I clinical trials in 56 patients with thyroid cancer: the M. D. Anderson Cancer Center experience. J Clin Endocrinol Metab 94: 4423-4432.
69. Ahn E Y, DeKelver R C, Lo M C, Nguyen T A, Matsuura S, et al. (2011) SON controls cell-cycle progression by coordinated regulation of RNA splicing. Mol Cell 42: 185-198.
70. Li X, Manley J L (2005) Inactivation of the S R protein splicing factor ASF/SF2 results in genomic instability. Cell 122: 365-378.
71. Li X, Wang J, Manley J L (2005) Loss of splicing factor ASF/SF2 induces G2 cell cycle arrest and apoptosis, but inhibits internucleosomal DNA fragmentation. Genes Dev 19: 2705-2714.
72. Terada Y, Yasuda Y (2006) Human immunodeficiency virus type 1 Vpr induces G2 checkpoint activation by interacting with the splicing factor SAP145. Mol Cell Biol 26: 8149-8158.
73. Kaida D, Schneider-Poetsch T, Yoshida M (2012) Splicing in oncogenesis and tumor suppression. Cancer Sci 103: 1611-1616.
74. Yoon S O, Shin S, Lee H J, Chun H K, Chung A S (2006) Isoginkgetin inhibits tumor cell invasion by regulating phosphatidylinositol 3-kinase/Akt-dependent matrix metalloproteinase-9 expression. Mol Cancer Ther 5: 2666-2675.
75. Pawitan Y, Bjohle J, Amler L, Borg A L, Egyhazi S, et al. (2005) Gene expression profiling spares early breast cancer patients from adjuvant therapy: derived and validated in two population-based cohorts. Breast Cancer Res 7: R953-964.
76. Sabatier R, Finetti P, Cervera N, Lambaudie E, Esterni B, et al. (2011) A gene expression signature identifies two prognostic subgroups of basal breast cancer. Breast Cancer Res Treat 126: 407-420.
77. Richardson A L, Wang Z C, De Nicolo A, Lu X, Brown M, et al. (2006) X chromosomal abnormalities in basal-like human breast cancer. Cancer Cell 9: 121-132.
78. Li Y, Zou L, Li Q, Haibe-Kains B, Tian R, et al. (2010) Amplification of LAPTM4B and YWHAZ contributes to chemotherapy resistance and recurrence of breast cancer. Nat Med 16: 214-218.
79. Johnson W E, Li C, Rabinovic A (2007) Adjusting batch effects in microarray expression data using empirical Bayes methods. Biostatistics 8: 118-127.
80. Kauffmann A, Gentleman R, Huber W (2009) arrayQualityMetrics—a bioconductor package for quality assessment of microarray data. Bioinformatics 25: 415-416.
81. Loi S, Haibe-Kains B, Desmedt C, Wirapati P, Lallemand F, et al. (2008) Predicting prognosis using molecular profiling in estrogen receptor-positive breast cancer treated with tamoxifen. BMC Genomics 9: 239.
82. Loi S, Haibe-Kains B, Desmedt C, Lallemand F, Tutt A M, et al. (2007) Definition of clinically distinct molecular subtypes in estrogen receptor-positive breast carcinomas through genomic grade. J Clin Oncol 25: 1239-1246.
83. Gong Y, Duvvuri M, Duncan M B, Liu J, Krise J P (2006) Niemann-Pick C1 protein facilitates the efflux of the anticancer drug daunorubicin from cells according to a novel vesicle-mediated pathway. J Pharmacol Exp Ther 316: 242-247.
84. Hutterer A, Berdnik D, Wirtz-Peitz F, Zigman M, Schleiffer A, et al. (2006) Mitotic activation of the kinase Aurora-A requires its binding partner Bora. Dev Cell 11: 147-157.
85. Niu N, Qin Y, Fridley B L, Hou J, Kalari K R, et al. (2010) Radiation pharmacogenomics: a genome-wide association approach to identify radiation response biomarkers using human lymphoblastoid cell lines. Genome Res 20: 1482-1492.
86. Rozenblum E, Vahteristo P, Sandberg T, Bergthorsson J T, Syrjakoski K, et al. (2002) A genomic map of a 6-Mb region at 13q21-q22 implicated in cancer development: identification and characterization of candidate genes. Hum Genet 110: 111-121.
87. Liang L, Qu L, Ding Y (2007) Protein and mRNA characterization in human colorectal carcinoma cell lines with different metastatic potentials; Cancer Invest 25: 427-434.
88. Lim J, Kuroki T, Ozaki K, Kohsaki H, Yamori T, et al. (1997) Isolation of murine and human homologues of the fission-yeast dis3+gene encoding a mitotic-control protein and its overexpression in cancer cells with progressive phenotype. Cancer Res 57: 921-925.
89. Chang S H, Chung Y S, Hwang S K, Kwon J T, Minai-Tehrani A, et al. (2012) Lentiviral vector-mediated shRNA against AIMP2-DX2 suppresses lung cancer cell growth through blocking glucose uptake. Mol Cells 33: 553-562.
90. Choi J W, Kim D G, Lee A E, Kim H R, Lee J Y, et al. (2011) Cancer-associated splicing variant of tumor suppressor AIMP2/p38: pathological implication in tumorigenesis. PLoS Genet 7: e1001351.
91. Komlosi V, Hitre E, Pap E, Adleff V, Reti A, et al. (2010) SHMT1 1420 and MTHFR 677 variants are associated with rectal but not colon cancer. BMC Cancer 10: 525.
92. Al-Sarraf N, Reiff J N, Hinrichsen J, Mahmood S, Teh B T, et al. (2007) DOK4/IRS-5 expression is altered in clear cell renal cell carcinoma. Int J Cancer 121: 992-998.
93. Park J H, Kim N S, Park J Y, Chae Y S, Kim J G, et al. (2010) MGMT —535G>T polymorphism is associated with prognosis for patients with metastatic colorectal cancer treated with oxaliplatin-based chemotherapy. J Cancer Res Clin Oncol 136: 1135-1142.
94. Joerger M, deJong D, Burylo A, Burgers J A, Baas P, et al. (2011) Tubulin, BRCA1, ERCC1, Abraxas, RAP80 mRNA expression, p53/p21 immunohistochemistry and clinical outcome in patients with advanced non small-cell lung cancer receiving first-line platinum-gemcitabine chemotherapy. Lung Cancer 74: 310-317.
95. Han M, Wang H, Zhang H T, Han Z (2012) The PDZ protein TIP-1 facilitates cell migration and pulmonary metastasis of human invasive breast cancer cells in athymic mice. Biochem Biophys Res Commun 422: 139-145.
96. Tomoda Y, Katsura M, Okajima M, Hosoya N, Kohno N, et al. (2009) Functional evidence for Eme1 as a marker of cisplatin resistance. Int J Cancer 124: 2997-3001.
97. Kim K, Heo K, Choi J, Jackson S, Kim H, et al. (2012) Vpr-binding protein antagonizes p53-mediated transcription via direct interaction with H3 tail. Mol Cell Biol 32: 783-796.
98. Brauweiler A, Lorick K L, Lee J P, Tsai Y C, Chan D, et al. (2007) RING-dependent tumor suppression and G2/M arrest induced by the TRC8 hereditary kidney cancer gene. Oncogene 26: 2263-2271.
99. Edgren H, Murumagi A, Kangaspeska S, Nicorici D, Hongisto V, et al. (2011) Identification of fusion genes in breast cancer by paired-end RNA-sequencing. Genome Biol 12: R6.

1. A computerized method of identifying candidate biomolecules relevant to a medical condition, the candidate biomolecules being putative clinical biomarkers for prognosis of, or putative therapeutic targets for treating, the medical condition, the method comprising:
for each subject k of a set of K subjects suffering from the medical condition, receiving subject data which indicates (i) for each gene pair i, j of a plurality of sense-antisense gene pairs (SAGPs), corresponding gene expression values y_{i,k}, y_{j,k} of subject k; and (ii) a survival time and survival event of subject k;
identifying, using said subject data, a prognostic subset of said SAGPs which optimally stratifies the subjects into low-risk and high-risk disease progression subgroups;
comparing gene expression values of each gene in the low-risk and high-risk subgroups which have been stratified by said prognostic subset of SAGPs, to identify a set of prognostic genes which are differentially expressed between the low-risk and high-risk subgroups; and
identifying one or more predefined biologically-related categories of genes which are over-represented in the set of differentially expressed prognostic genes, wherein the candidate biomolecules comprise genes or gene products belonging to said over-represented categories.

2. A computerized method according to claim 1, wherein the set of K subjects comprises a plurality of independent cohorts of subjects.

3. A computerized method according to claim 2, wherein said differentially expressed prognostic genes are identified by:
for each cohort, identifying a cohort-specific set of genes which is differentially expressed in said cohort, to thereby obtain a plurality of cohort-specific sets; and
finding the intersection of the cohort-specific sets to obtain the set of differentially expressed genes.

4. A computerized method according to any one of claims 1 to 3, wherein genes in respective predefined categories of biologically-related genes are related by one or more of:
cellular localization, biological process, molecular function, or biological pathway.

5. A computerized method according to any one of the preceding claims, wherein identifying the prognostic subset of SAGPs comprises:
generation of a statistical partition model (SPM) for each of each SAGPs using said subject data;
obtaining data characterizing the statistical significance of the SPMs; and
identifying of a subset of said SAGPs using the data characterizing the statistical significance.

6. A computerized method according to claim 5,
the method comprising for each SAGP:
(i) defining a plurality of trial values for each of two cut-off values cⁱ and c^{j};
(ii) for each of a plurality of angles α, for each subject, and for each of the trial cut-off values cⁱ and c^{j}:
(a) comparing the expression values to a respective pair of lines in a two-dimensional space spanned by the expression values to obtain comparison data indicating on which side of the pair of lines the expression values for the corresponding subject lie, the pair of lines being formed using the cut-off values cⁱ and c^{j}, each of the lines having angle α to a direction in the space indicating increasing values of a corresponding one of the expression values; and
(b) generating at least one SPM based on the comparison data; and
(iii) selecting the one of the SPMs (‘the maximally predictive SPM’) which has the maximal statistical value in predicting the survival times of the subjects.

7. A computerized method according to claim 6 in which for each of the plurality of angles α, and for each subject, and for each of the trial cut-off values cⁱ and c^{j}, a plurality of statistical partition models of survival prognosis of the patients are constructed based on a plurality of respective designs, each design representing a respective combination of possibilities for realizations of the comparison data.

8. A computerized method according to claim 7 in which the comparison data for a given subject, a given angle α, a given said subject, and a given pair of trial cut-off values cⁱ and c^{j}, takes one of four possibilities:
A: indicating that both the corresponding expression values lie on a first side of the lines;
B: indicating that a first of the expression values lies on the first side of a first of the lines, and the second value lies on a second side of the second of the lines;
C: indicating that the first of the expression values lies on a second side of the first of the lines, and the second value lies on the first side of the second of the lines; and
D: indicating that both expression values lie on the second side of the lines;
and the plurality of designs include:
a first design indicating whether the subjects' expression level values are within regions A or D, rather than B or C;
a second design indicating whether the subjects' expression level values are within regions A, B or C, rather than D;
a third design indicating whether the subjects' expression level values are within regions A, C or D, rather than B;
a fourth design indicating whether the subjects' expression level values are within regions B, C or D, rather than A;
a fifth design indicating whether the subjects' expression level values are within regions A, B or D, rather than C;
a sixth design indicating whether the subjects' expression level values are within regions A or C, rather than B or D;
a seventh design indicating whether the subjects' expression level values are within regions A or B, rather than C or D.

9. A computerized method according to any of claims 6 to 8, comprising selecting the subset of the gene pairs for which the corresponding selected models are of maximal statistical significance of the survival prognosis model.

10. A computerized method according to claim 9 further including i) a step of determining for each gene of the selected gene pairs the statistical significance of the expression level of the individual genes of the survival prognosis model, and ii) a step of selecting of the gene pairs for which the statistical significance of the maximally predictive SPM is higher than a threshold of the statistical significance of the individual genes of the gene pair.

11. A computerized method of clinical outcome prognosis in a subject having a medical condition, the method comprising:
receiving data representing parameters of one or more statistical partition models (SPMs) said SPMs being configured to stratify a cohort of subjects having the medical condition into subgroups, said parameters representing, for each gene pair of one or more sense-antisense gene pairs (SAGPs), a pair of lines in a two-dimensional space spanned by respective expression level values of respective genes i, j in the gene pair, the pair of lines being formed using two cut-off values cⁱ and c^{j}, and each of the lines having a non-zero angle α to each of two axis directions in the space indicating increasing values of a corresponding one of the expression level values;
receiving expression level data representing expression levels in the subject of genes of one or more selected SAGPs; and
for each SAGP of the selected SAGPs, comparing the expression levels to the pair of lines for the SAGP to obtain comparison data indicating on which side of the pair of lines the expression values for the subject lie, thereby obtaining a prediction of a subgroup to which the subject belongs.

12. A computerized method according to claim 11, wherein the SAGPs comprise one or more of the gene pairs listed in Table 1A.

13. A computerized method according to claim 11 or claim 12, wherein the medical condition is breast cancer, colon cancer or non-small cell lung cancer, and wherein the SAGPs comprise one or more of the gene pairs listed in Table 1B.

14. A computerized method according to any one of claims 11 to 13, wherein there are two or more selected SAGPs, and wherein the method comprises combining the predictions of the subgroups from the two or more SAGPs to obtain a composite prediction.

15. A computerized method according to claim 14, wherein each prediction is represented by a group index, and wherein the predictions are combined by computing a weighted sum of the group indices.

16. A computerized method according to claim 15, wherein weights of the weighted sum are generated from p-values of respective SPMs corresponding to the selected SAGPs.

17. A kit for predicting clinical outcome in a subject having a medical condition, the kit comprising: a plurality of polynucleotide sequences, ones of the plurality of polynucleotide sequences being capable of specifically hybridizing to and/or detecting a gene of a plurality of genes and/or an expression product of the gene to obtain respective gene expression values, wherein the plurality of genes comprises one or more of the sense-antisense gene pairs (SAGPs) listed in Table 1A, and written instructions for comparing, and/or a tangible computer-readable medium having stored thereon machine-readable instructions for causing a computer processor to compare, the respective gene expression values to optimal gene expression cut-off values, wherein the plurality of genes comprises no more than 100 genes; and wherein the optimal gene expression cut-off values are determined for each SAGP by:
(i) defining a plurality of trial values for each of two cut-off values cⁱ and c^{j};
(ii) for each of a plurality of angles α, for each subject, and for each of the trial cut-off values cⁱ and c^{j}:
(a) comparing the expression values to a respective pair of lines in a two-dimensional space spanned by the expression values to obtain comparison data indicating on which side of the pair of lines the expression values for the corresponding subject lie, the pair of lines being formed using the cut-off values cⁱ and c^{j}, each of the lines having angle α to a direction in the space indicating increasing values of a corresponding one of the expression values; and
(b) generating at least one SPM based on the comparison data; and
(iii) selecting the one of the SPMs (‘the maximally predictive SPM’) which has the maximal statistical value in predicting the survival times of the subjects,
whereby the cut-off values cⁱ and c^{j} for the maximally predictive SPM are the optimal gene expression cut-off values.

18. A kit according to claim 17, wherein the plurality of genes comprises the sense-antisense gene pairs listed in Table 1A.

19. A kit according to claim 17, wherein the plurality of genes comprises the sense-antisense gene pairs listed in Table 1B.

20. A kit according to any one of claims 17 to 19, wherein the polynucleotide sequences are immobilized on a solid support.

21. A kit according to any one of claims 17 to 20, comprising at least one primer for amplification of one or more of the plurality of genes, or at least part thereof.

22. A kit according to claim 21, wherein the primers are selected from the primers listed in Table 9.

23. A computerized method of composite survival prediction combining the output values from a plurality of SPMs associated with prognosis of a potentially fatal medical condition in each subject k of a set of K subjects suffering from the medical condition, each SPM being a model of the statistical significance of the expression level values of a corresponding set of one or more genes or gene pairs, the method employing test data which for each gene i of the pair of genes indicates a corresponding gene expression value y_{i,k} of subject k;
the method including:
for each subject obtaining for each of the SPMs a respective risk level value indicative of a risk level for the subject;
forming a weighted average of the risk level values using a set of respective weights, the weights being indicative of the relative quality of patient separation according to the given SPM versus others of the respective models in context of statistical significance of the relative risk statistics of the medical condition;
comparing the weighted average with a cut-off value to obtain a prognosis value.

26. A computerized method according to any one of claims 23 to 25 in which each of said models is a SPM of an individual gene or a gene pair.

27. A computerized method according to any of claims 23 to 26 in which each of said models is a SPM of a pair of genes obtained by a method according to claim 6 or any claim dependent therefrom.

28. A computerized method according to any one of claims 11 to 16, wherein the medical condition is Estrogen Receptor positive (ER“+”), Lymph Node negative (LN“−”) breast cancer, and wherein the subject has received adjuvant systemic tamoxifen treatment upon or after curative surgery.

29. A computerized method according to claim 28 in which the selected gene pair is or the selected gene pairs include the RNF139/TATDN1 SAGP.

30. A computerized method according to any one of claims 11 to 15, wherein the medical condition is a grade 3 breast tumor.

31. A computerized method according to claim 30 in which the selected gene pair is or the selected gene pairs include the VPRBP/RBM15B SAGP.

32. A computerized method according to any one of claims 11 to 16, wherein the medical condition is a grade 3 or grade 3-like breast tumor.

33. A computerized method according to claim 32 in which the selected gene pair is or the selected gene pairs include the C18orf8/NPC1 and/or the EME1/LRRC59 SAGP.

34. A computerized method according to any one of claims 11 to 15, wherein the medical condition is a grade 1 or grade 1-like breast tumor.

35. A computerized method according to claim 34 in which the selected gene pair is or the selected gene pairs include the SHMT1/SMCR8 SAGP.

36. A computerized method according to any one of claims 11 to 16, wherein the medical condition is a grade 1 breast tumor.

37. A computerized method according to any one of claims 11 to 16, wherein the medical condition is Estrogen Receptor negative (ER“−”) breast cancer.

38. A computerized method according to claim 37 in which the selected gene pair is or the selected gene pairs include the CTNS/TAX1BP3 SAGP.

39. A computerized method according to any one of claims 11 to 16, wherein the medical condition is a basal-like grade 3 (G3) breast tumor.

40. A computerized method according to claim 39 in which the selected gene pair is or the selected gene pairs include the CTNS/TAX1BP3 and/or the RNF139/TATDN1 SAGP.

41. A computerized method according to any one of claims 11 to 16, wherein the medical condition is a Luminal A breast tumor.

42. A computerized method according to claim 41 in which the selected gene pair is or the selected gene pairs include the BIVM/KDELC1 SAGPs.

43. A computerized method according to any one of claims 11 to 16, wherein the medical condition is ER“+”, LN“−”, Progesterone Receptor positive (PgR“+”) breast cancer and the subject has a breast tumor <=2 cm.

44. A method of prognosis of survival or treatment response in a subject suffering from breast cancer, comprising:
obtaining a test sample from the subject;
measuring a gene expression level in the test sample for one or more of the prognostic genes obtained according to claims 1 to 4 and listed in Table 11; and
comparing the measured gene expression level to a predefined threshold;
wherein a measured gene expression level which is above the predefined threshold is indicative of a poor prognosis.

45. A method according to claim 44, wherein the one or more genes comprises one or more of the genes listed in Table 10.

46. A method according to claim 44 or claim 45, wherein said measuring comprises contacting with the sample at least one nucleic acid probe capable of specifically hybridizing to the one or more genes or part thereof.

47. A kit for prognosis of survival or treatment response in a subject having breast cancer, the kit comprising: at least one nucleic acid probe capable of specifically hybridizing to and/or detecting a gene of a plurality of genes and/or an expression product of the gene, wherein the plurality of genes comprises one or more of the genes listed in Table 11, and wherein the plurality of genes comprises no more than 200 genes.

48. A kit according to claim 47, wherein the plurality of genes comprises the genes listed in Table 11.

49. A kit according to claim 47, wherein the plurality of genes comprises the genes listed in Table 10.

50. A kit according to any one of claims 47 to 49, wherein the nucleic acid probe or probes is or are immobilized on a solid support.

51. A kit according to any one of claims 47 to 50, comprising at least one primer for amplification of one or more of the plurality of genes, or part thereof.

52. A system for identifying candidate biomolecules relevant to a medical condition, the candidate biomolecules being putative clinical biomarkers for prognosis of, or putative therapeutic targets for treating, the medical condition; or for clinical outcome prognosis in a subject having a medical condition; or for composite survival prediction combining the output values from a plurality of SPMs associated with prognosis of a potentially fatal medical condition; or for prognosis of survival or treatment response in a subject suffering from breast cancer; the system comprising: at least one processor; and a tangible computer-readable storage medium having stored thereon machine-readable instructions for causing the at least one processor to perform the method according to any one of claims 1 to 16 or 23 to 46.
